(19) United States
(12) Patent Application Publication
(10) Pub. No.: US 2005/0234239 A1
(43) Pub. Date: Oct. 20, 2005

(54) Method of forming a carbon-carbon or carbon-heteroatom linkage

(76) Inventors: Marc Taillefer, Vailhauques (FR); Henri-Jean Cristau, Saint Aunes (FR); Pascal-Philippe Cellier, Paris (FR)

Correspondence Address: Jean-Louis Seugnet;Rhodia Inc Intellectual Property Dept, 259 Prospect Plains Road, CN 7500, Cranbury, NJ, 08512-7500, US

(21) Appl. No.: 10/515,856

(22) PCT Filed: Jun. 2, 2003
(86) PCT No.: PCT/FR03/01647

§ 371 Date: May 18, 2005

(30) Foreign Application Priority Data
Mar. 31, 2002 (FR) 02/6717

(51) Int. Cl. C07F 1/08; C07D 41/14

(52) U.S. Cl. 546/2; 546/256

(57) ABSTRACT

The invention relates to a method of creating a carbon-carbon or carbon-heteroatom linkage by reacting an unsaturated compound bearing a leaving group and a nucleophilic compound. More specifically, the invention relates to the creation of a carbon-nitrogen linkage involving the arylation of nitrogenous organic derivatives. The inventive method consists in creating a carbon-carbon or carbon-heteroatom linkage by reacting an unsaturated compound bearing a leaving group and a nucleophilic compound providing a carbon atom or a heteroatom (HE) capable of being substituted for the leaving group, thereby creating a C—C or C-HE linkage. The invention is characterised in that the reaction is carried out in the presence of an effective quantity of a catalyst based on copper and at least one ligand comprising at least one imine function and at least one additional nitrogen atom as chelating atoms

The present invention relates to a process for creating a carbon-carbon or carbon-heteroatom bond by reacting an unsaturated compound carrying a leaving group and a nucleophilic compound.

More particularly, the invention relates to creating a carbon-nitrogen bond using a process for arylating organic nitrogen-containing derivatives.

Many important compounds exist in the agrochemical and pharmaceutical fields, for example arylhydrazines, which result from arylating a nucleophilic compound by creating a carbon-nitrogen bond.

A conventional arylation method consists of carrying out the Ullmann reaction (Ullmann F. and Kipper H., Ber. Dtsch. Chem. Ges. 1905, 38, 2120-2126), by prolonged heating of the reagents at high temperature, in the presence of catalytic or stoichiometric copper. The reactions are usually limited to using aryl iodides and their yields are reduced by competitive formation of biaryl homocoupling products.

Arylation reactions require a catalyst; a number of types of catalyst have been described.

Palladium was used by Buchwald et al., in particular to carry out indole arylation (Org. Lett. 2000, 2, 1403-1406), in the presence of a base in toluene at 80° C.-100° C. Generally, the yields are satisfactory, but the reaction temperature is still high for this type of palladium-based catalyst.

Copper has also been used (Chiriac et al., Rev. Roum. Chim. 1969, 14, 1263-1267) to carry out arylation of sodium salts and pyrazoles by iodobenzene in the presence of a catalytic quantity of copper under DMF reflux. The conditions described are very severe, the temperature is 153° C. and the reaction period is very long at 30 to 40 hours.

Beletskaya et al. (Tetrahedron Lett. 1998, 39, 5617-5622) proposed a combination of palladium and copper when N-arylating benzotriazole. The presence of copper is indispensable to controlling the selectivity of the reaction. The catalyst is a phase transfer catalyst which is not easy to use on an industrial scale.

International patent WO-A-98/00399 proposes the use of a nickel catalyst, but this has proved to be of little effect when arylating heterocycles such as imidazole.

Chan et al. also described (J. Chem. RES. (S) 2000, 367-369) the arylation of azoles from diaryliodonium salts in the presence of a cobalt catalyst under phase transfer conditions.

Buchwald et al. (J. Am. Chem. Soc. 2001, 123, 7727-7729) recently developed a method for arylating nitrogen-containing nucleophiles catalysed by copper. Its catalytic system, composed of a catalyst that is insensitive to air, cuprous iodide and the trans-1,2-diaminocyclohexane ligand, allows heterocycles such as pyrazoles, indoles, carbazole, pyrrole, indazole, imidazole, phthalazinone and 7-azaindole to be arylated in dioxane at 110° C.

The disadvantage of that process is that the temperature is still high when arylation is carried out by aryl chlorides or even by aryl iodides.

The present invention aims to provide a process that overcomes the disadvantages cited above and which is applicable to a very large number of nucleophiles.

We have now discovered, and this constitutes the subject matter of the present invention, a process for creating an unsaturated compound carrying a leaving group and a nucleophilic compound carrying a carbon atom or a heteroatom (HE) that can substitute for the leaving group, thus creating a C—C or C-HE bond, characterized in that the reaction takes place in the presence of an effective quantity of a copper-based catalyst and at least one ligand comprising at least one imine function and at least one supplemental nitrogen atom as chelating atoms.

In a variation of the process of the invention, the arylation reaction is carried out by reacting an aromatic compound carrying a leaving group and a nucleophilic compound.

In a further variation of the process of the invention, a vinylation or alkynation reaction is carried out by reacting a compound having a double or triple bond in a position a to a leaving group and a nucleophilic compound respectively.

Throughout the description of the present invention, the term “arylation” is used in its broad sense since it is envisaged that the compound employed carries a leaving group which is either of the unsaturated aliphatic type, or of the carbocyclic aromatic or heterocyclic type.

The term “nucleophilic compound” means an organic hydrocarbon compound that may be acyclic or cyclic and comprises at least one atom carrying a free electron pair which may or may not carry a charge, preferably a nitrogen, oxygen, sulphur, phosphorus or carbon atom.

The term “imine function” means a functional group comprising a nitrogen atom bonded to a carbon atom via a double bond.

The term “other supplemental nitrogen atom” means a nitrogen atom that can be carried by a further imine function and/or by a functional group such as an amine, amide, urea, nitrile, guanidine, sulphonamide, phosphinamide group and/or a nitrogen atom carrying a free electron pair included in a saturated, unsaturated or aromatic cycle.

As mentioned above, the nucleophilic compound comprises at least one atom carrying a free electron pair, which can be carried by a functional group and/or a carbanion.

Examples of functional groups comprising said atoms that can be mentioned are:

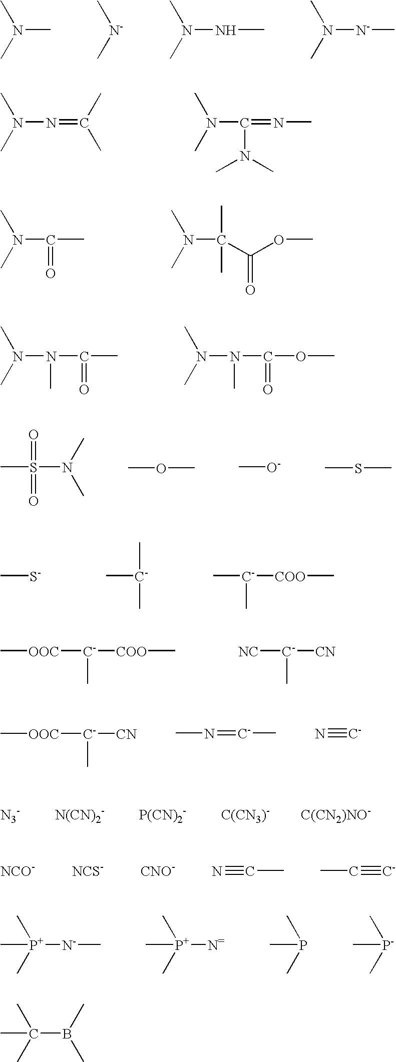

In a further variation of the invention, the nucleophilic compound comprises at least one nitrogen atom carrying a free electron pair included in a saturated, unsaturated or aromatic cycle; the cycle generally contains 3 to 8 atoms.

It should be noted that when the nucleophilic compound comprises a functional group, examples of which were given above, which carries one or more negative charges, said compound is then in its salt form. The counter-ion is generally a metallic cation such as an alkali metal, preferably sodium or lithium, or an alkaline-earth metal, preferably calcium, or the residue of an organometallic compound such as a magnesium or zinc compound.

A first advantage of the process of the invention is that it is carried out at moderate temperatures.

A further advantage is that a wide range of arylation agents for nucleophiles can be used, not only aryl iodides, but also aryl bromides.

A still further advantage of the process of the invention is the possibility of using copper rather than palladium as the catalyst, bringing an additional economic advantage.

In accordance with the process of the invention, the catalyst is associated with a ligand which is polydentate, at least bidentate, tridentate or even tetradentate, and which comprises the atoms defined above in the description of the invention.

Examples of ligands will be shown below in formulae which are given by way of example and are not limiting in nature.

The ligands comprise at least one imine function. Advantageously, the imine function is not included in a cycle.

A first category of ligands for carrying out the process of the invention is constituted by hydrazone type ligands, in particular those with formula: 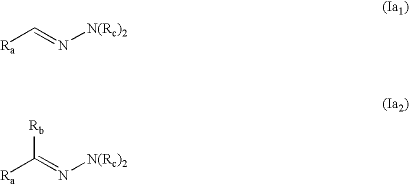
in which formulae:
one of groups Rₐ and R_{b} can comprise at least one nitrogen atom or a group comprising a nitrogen atom;
Rₐ and R_{b} independently represent a hydrocarbon group containing 1 to 20 carbon atoms, which may be a linear or branched, saturated or unsaturated, acyclic aliphatic group; a monocyclic or polycyclic, saturated, unsaturated or aromatic carbocyclic or heterocyclic group; or a concatenation of said groups;
or Rₐ and R_{b} can be bonded to constitute, with the carbon atoms carrying them, a monocyclic or polycyclic, saturated or unsaturated carbocyclic or heterocyclic group containing 3 to 20 atoms;
at most one of groups Rₐ and R_{b} represents a hydrogen atom;
R_{c}, which may be identical or different, represents a hydrogen atom, an alkyl group, preferably C₁ to C₁₂; an alkenyl or alkynyl group, preferably C₂ to C₁₂; a cycloalkyl group, preferably C₃ to C₁₂; an aryl or arylalkyl group, preferably C₆ to C₁₂, an amido group —CO—NH₂; an amido group substituted with one or two alkyl groups, preferably C₁ to C₁₂; and/or an alkenyl or alkynyl group, preferably C₂ to C₁₂; and/or a cycloalkyl group, preferably C₃ to C₁₂; and/or an aryl or arylalkyl group, preferably C₆ to C₁₂.

As mentioned above, at least one of groups Rₐ and R_{b} comprises a nitrogen atom or a group containing a nitrogen atom; examples that can be cited are groups such as amino, amido, . . . . The NH₂ group is preferred.

In formulae (Ia₁) and (Ia₂), the different symbols can in particular have the meanings given below.

Thus, Rₐ and R_{b} can independently represent a linear or branched, saturated or unsaturated, acyclic aliphatic group.

More precisely, Rₐ and R_{b} preferably represent a linear or branched, saturated acyclic aliphatic group, preferably C₁ to C₁₂, and more preferably C₁ to C₄.

The invention does not exclude the presence of an unsaturated bond on the hydrocarbon chain such as one or more double bonds, which may or may not be conjugated.

The hydrocarbon chain can optionally be interrupted by a heteroatom (for example oxygen, sulphur, nitrogen or phosphorus) or by a functional group provided that it does not react, in particular, a group such as —CO—.

The hydrocarbon chain can optionally carry one or more substituents (for example halogen, ester, amino or alkyl and/or arylphosphine) provided that they do not interfere.

The linear or branched, saturated or unsaturated acyclic aliphatic group can optionally carry a cyclic substituent. The term “cycle” means a saturated, unsaturated or aromatic carbocyclic or heterocyclic cycle.

The acyclic aliphatic group can be connected to the cycle via a covalent bond, a heteroatom or a functional group such as oxy, carbonyl, carboxyl, sulphonyl, etc. . . .

Examples of cyclic substituents that can be envisaged are cycloaliphatic, aromatic or heterocyclic substituents, in particular cycloaliphatic substituents containing 6 carbon atoms in the cycle or benzenic, said cyclic substituents themselves optionally carrying any substituent provided that they do not interfere with the reactions occurring in the process of the invention. Particular mention can be made of C₁ to C₄ alkyl or alkoxy groups.

More particular aliphatic groups carrying a cyclic substituent include cycloalkylalkyl groups, for example cyclohexylalkyl, or arylalkyl groups, preferably C₇ to C₁₂, in particular benzyl or phenylethyl.

In group formulae (Ia₁) and (Ia₂), groups Rₐ and R_{b} can also independently represent a carbocyclic group that is saturated or contains 1 or 2 unsaturated bonds in the cycle, generally C₃ to C₈, preferably with 6 carbon atoms in the cycle; said cycle can be substituted. A preferred example of this type of group that can be cited is cyclohexyl, optionally substituted with linear or branched alkyl groups containing 1 to 4 carbon atoms.

Groups Rₐ and R_{b} can independently represent an aromatic hydrocarbon group, in particular benzenic with general formula (F₁): 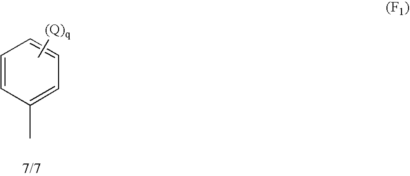
in which:
   q represents a whole number from 0 to 5;
   Q is a group selected from a linear or branched C₁ to C₆ alkyl group, a linear or branched C₁ to C₆ alkoxy group, a linear or branched C₁ to C₆ alkylthio group, a —NO₂ group, a —CN group, a halogen atom or a CF₃ group.

Thus, the aromatic hydrocarbon group can be substituted. Q illustrates certain types of preferred substituents, but enumeration is not limiting.

Rₐ and R_{b} can also independently represent a polycyclic aromatic hydrocarbon group with cycles that can between them form ortho-condensed or ortho- and peri-condensed systems. A more particular example that can be cited is a naphthyl group; said cycle can be substituted.

Rₐ and R_{b} can also independently represent a polycyclic hydrocarbon group constituted by at least 2 saturated and/or unsaturated carbocycles or by at least 2 carbocycles only one of which is aromatic and forming ortho- or ortho- and peri-condensed systems between them. Generally, the cycles are C₃ to C₈, preferably C₆. More particular examples that can be cited are the bornyl group and the tetrahydronaphthalene group.

Rₐ and R_{b} can also independently represent a saturated, unsaturated or aromatic heterocyclic group in particular containing 5 or 6 atoms in the cycle, including one or two heteroatoms such as nitrogen atoms (not substituted with a hydrogen atom), sulphur or oxygen; the carbon atoms of this heterocycle can also be substituted.

Rₐ and R_{b} can also represent a polycyclic heterocyclic group defined as either a group constituted by at least two aromatic or non aromatic heterocycles containing at least one heteroatom in each cycle and forming ortho- or ortho- and peri-condensed systems between them, or a group constituted by at least one aromatic or non aromatic hydrocarbon cycle and at least one aromatic or non aromatic heterocycle forming between them ortho- or ortho- and peri-condensed systems; the carbon atoms of said cycles can optionally be substituted.

Examples of heterocyclic type groups Rₐ and R_{b} that can be cited include furyl, thienyl, isoxazolyl, furazannyl, isothiazolyl, pyridyl, pyridazinyl, pyrimidinyl, pyrannyl, phosphino and quinolyl, naphthyridinyl, benzopyrannyl or benzofurannyl groups.

The number of substituents present on each cycle depends on the carbon condensation of the cycle and on the presence or otherwise of an unsaturated bond on the cycle. The maximum number of substituents that can be carried by a cycle can readily be determined by the skilled person.

Rₐ and R_{b} can be connected to constitute, with the carbon atoms carrying them, a monocyclic or polycyclic, saturated, unsaturated or aromatic carbocyclic or heterocyclic group containing 3 to 20 atoms, comprising two or three cycles; the adjacent cycles can be aromatic in nature. In the case of polycyclic compounds, the number of atoms in each cycle is preferably in the range 3 to 6. Rₐ and R_{b} preferably form a cyclohexane or fluorenone cycle.

In formulae (Ia₁) and (Ia₂) for hydrazone type ligands, groups R_{c} preferably represent a hydrogen atom or a C₁-C₄ alkyl group, an amido group, or an amido group substituted with a C₁-C₄ alkyl group.

Preferred hydrazone type ligands have formula (Ia₁) or (Ia₂) in which Rₐ and R_{b} preferably represent one of the following groups with formula (F₀): 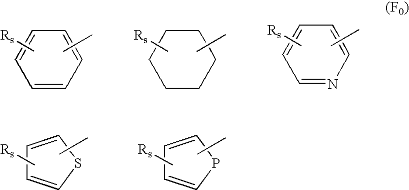
in which R₅ represents a hydrogen atom, an alkyl or alkoxy group, preferably C₁ to C₄, or an amino or amido group which may or may not be substituted with an alkyl group, preferably C₁ to C₄, or a phosphino group substituted with alkyl groups, which may be identical or different, preferably C₁ to C₄, or with phenyl groups.

Preferred groups with formula (F₀) are those with formula (F₄): 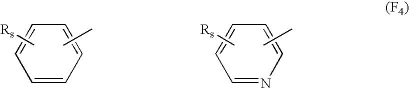
in which R₅ represents a hydrogen atom, an alkyl or alkoxy group, preferably C₁ to C₄, or an amino or amido group which may or may not be substituted with an alkyl group, preferably C₁ to C₄.

In the pyridyl group, the bond is advantageously located in the position ortho to the nitrogen atom.

Preferred hydrazone type ligands for use in the process of the invention are those with formula (Ia₁) or (Ia₂) in which groups R_{c}, which may be identical or different, represent a hydrogen atom or a methyl group, and Rₐ represents one of the following groups with formula (F₀), preferably (F₄).

Preferred hydrazone type ligands are those with formula (Ia₁).

Hydrazone type ligands are produced by reacting:
an aldehyde or ketone with the following formulae: 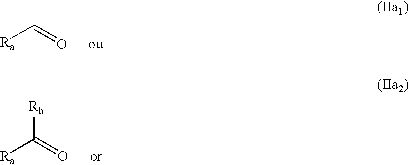
   in which formulae (IIa₁) or (IIa₂), Rₐ and R_{b} have the meanings given in formulae (Ia₁) or (Ia₂);
with a hydrazine or derivative with formula (IIa₃), preferably hydrazine, N-methylhydrazine or N,N-dimethylhydrazine: 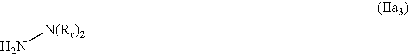
   in which formula (IIa₃), R_{c}, which may be identical or different, have the meanings given in formulae (Ia₁) or (Ia₂).

Preferred hydrazone type ligands used in the process of the invention contain a nitrogen atom supplied by the pyridyl group of a pyridylaldehyde residue. They are preferably obtained by reacting a pyridylaldehyde with a hydrazine or a N-substituted or N,N-disubstituted hydrazine, preferably substituted with an alkyl group containing 1 to 4 carbon atoms.

Examples of preferred ligands are given below:

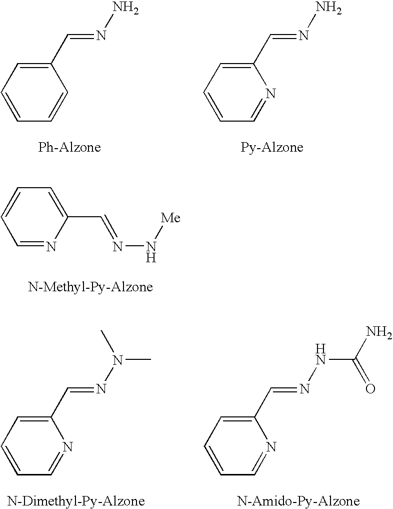

A further category of ligands that is suitable for carrying out the invention is formed by tetradentate ligands: 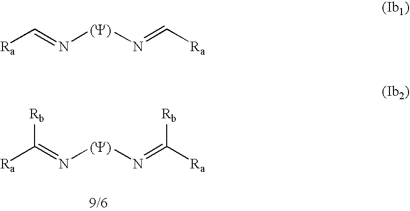
in which formulae:
   Rₐ, which may be identical or different, have the meanings given in formulae (Ia₁) and (Ia₂);
   R_{b}, which may be identical or different, have the meanings given in formulae (Ia₁) and (Ia₂);
   ψ represents a covalent bond, a urea group or a skeleton with general formula (F₂) or (F₃): 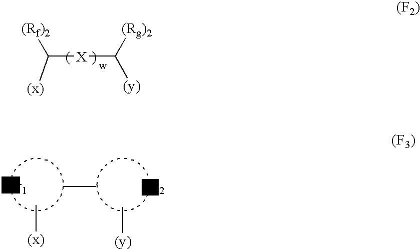
   in which formulae (F₂) and (F₃):
      R_{f} and R_{g}, which may be identical or different, independently represent a hydrogen atom, a hydrocarbon group containing 1 to 20 carbon atoms, which may be a linear or branched, saturated or unsaturated acyclic aliphatic group; a monocyclic or polycyclic, saturated, unsaturated or aromatic carbocyclic or heterocyclic group; or a concatenation of said groups;
      or R_{f} and R_{g} can be bonded together to constitute, with the carbon atoms carrying them, a carbocyclic or heterocyclic group containing 3 to 20 atoms, which may be saturated, unsaturated, monocyclic or polycyclic;
      Ar₁ and Ar₂ independently represent two substituted or non substituted aromatic, carbocyclic or heterocyclic cycles which may or may not be condensed, which may carry one or more heteroatoms;
      X represents a methylene group, which may be substituted;
      w is a whole number from 0 to 3; and
      x and y respectively represent the two bonds between the skeleton shown as ψ and the imine groups.

In formulae (Ib₁) and (Ib₂), symbols Rₐ and R_{b} can have the meanings given for formulae (Ia₁) and (Ia₂).

Preferred tetradentate ligands have formulae (Ib₁) or (Ib₂) in which Rₐ and R_{b} represent one of the groups with formula (F₀).

Preferred groups with formula (F₀) are those with one of the following formulae (F₅): 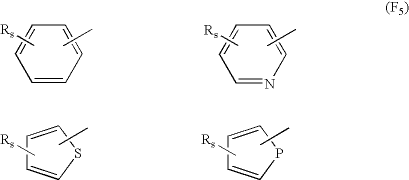
in which R₅ represents a hydrogen atom, an alkyl or alkoxy group, preferably C₁ to C₄, or an amino or amido group which may or may not be substituted with alkyl groups, preferably C₁ to C₄.

Preferred tetradentate type ligands are those with formula (Ib₁).

In formulae (F₂) and (F₃), symbols R_{f} and R_{g} can have the meanings given for Rₐ and R_{b} in formulae (Ia₁) and (Ia₂).

Preferably, R_{f} is identical to R_{g}.

Further, R_{f} and R_{g} can also be bonded together to constitute, with the carbon atoms which carry them, a saturated, unsaturated or aromatic, monocyclic or polycyclic carbocyclic or heterocyclic group. R_{f} and R_{g} preferably form a cyclohexane or benzene type cycle.

Illustrative examples of group ψ that can be mentioned are the following cyclic groups:

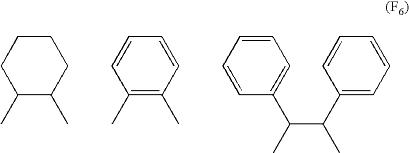

Particularly advantageous compounds have general formula (F₂) in which:
R_{f} and R_{g} both represent a phenyl or naphthyl group;
R_{f} and R_{g} are bonded together to constitute a cycle such as cyclohexane or benzene with the carbon atoms carrying them.

In formula (F₃), Ar₁ and Ar₂ together represent an aromatic group which can be a carbocycle containing 6 to 12 carbon atoms or a heterocycle containing 5 to 12 atoms.

In the following description of the present invention, the term “aromatic” designates the conventional idea of aromaticity as defined in the literature, in particular J. March, “Advanced Organic Chemistry”, 4^{th} edition, John Wiley & Sons, 1992, pp. 40 ff.

Within the context of the present invention, the aromatic derivative can be monocyclic or polycyclic.

In the case of a monocyclic derivative, it can comprise one or more heteroatoms in its cycle selected from nitrogen, phosphorus, sulphur and oxygen atoms. A preferred mode uses nitrogen atoms not substituted with a hydrogen atom.

Illustrative examples of monocyclic heteroaromatic derivatives that are suitable for use in the present invention that can be cited are pyridine, pyrimidine, pyridazine and pyrazine derivatives.

The carbon atoms of the aromatic derivative can also be substituted. Two neighbouring substituents on the aromatic cycle can also, together with the carbon atoms carrying them, form a hydrocarbon cycle, preferably aromatic, and can if necessary comprise at least one heteroatom. The aromatic group is then a polycyclic group.

Illustrative examples of this type of compound that can be cited are naphthalene derivatives, quinoline derivatives and isoquinoline derivatives.

Representative examples of compounds with general formula (F₃) that can in particular be cited are those in which Ar₁ and Ar₂ together form either a group deriving from a diphenyl-2,2′-diyl group, or a dinaphthyl-2,2′-diyl group.

The following cyclic groups constitute illustrative examples of groups ψ:

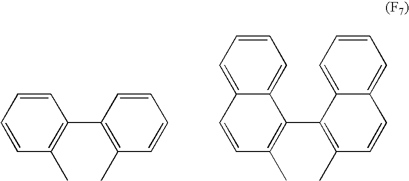

Tetradentate type ligands which are preferentially used in the process of the invention are those with formula (Ib₁) in which ψ represents a covalent bond, a urea group or one of groups (F₆) or (F₇) and Rₐ represents a group with formula (F₀), preferably (F₅).

Preferably, the invention does not envisage the use of 1,2-bis-(4-dimethylaminobenzylideneamino)ethane.

Ligands with formulae (Ib₁) or (Ib₂) are known products.

They are obtained by reacting:
an aldehyde or ketone with the following formulae: 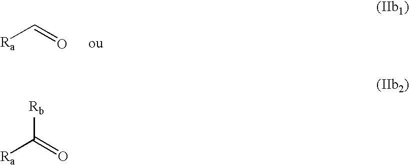
   in which formulae (IIb₁) or (IIb₂), Rₐ and R_{b} have the meanings given in formulae (Ia₁) or (Ia₂);
with a diamine or with formula (IIb₃):
   H₂N-ψ-NH₂ (IIb₃)
   in which formula (IIb₃), ψ has the meaning given in formulae (Ib₁) or (Ib₂) and represents a covalent bond, a urea group or a skeleton with general formula (F₂) or (F₃).

Preferred tetradentate type ligands used in the process of the invention contain a nitrogen atom carried by the pyridyl group of a pyridylalkdehyde residue. They preferably result from reacting pyridylaldehyde with urea, 1,2-cyclohexanediamine or 1,2-diphenylethylenediamine.

Examples of preferred ligands are given below:

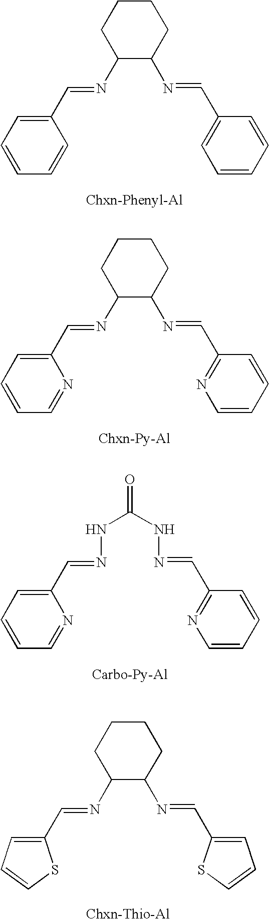

A further category of ligands that can be used in the invention is formed by bidentate ligands with formula: 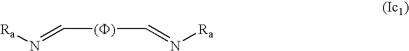
in which formula:
   Rₐ, which may be identical or different, have the meanings given in formulae (Ia₁) and (Ia₂);
   Φ represents:
      a covalent bond; 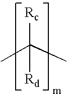
      an alkylene group with formula:
         in which R_{c}, R_{d}, which may be identical or different, represent:
         a hydrogen atom;
         a linear or branched alkyl group containing 1 to 12 carbon atoms, optionally carrying a halogen atom, preferably 1 to 4 carbon atoms, such as methyl, ethyl, propyl, isopropyl, butyl, isobutyl, sec-butyl or tert-butyl;
         a halogen atom;
         and m equals 0, 1 or 2, preferably 0 or 1;
      or the residue of a saturated, unsaturated or aromatic, monocyclic or polycyclic hydrocarbon cycle containing 5 to 12 carbon atoms carrying the two imine functions in the ortho or meta position.

Preferred bidentate type ligands have formula (Ic₁) in which groups Rₐ represent one of the following groups of the groups with formula (F₀).

Preferred groups with formula (F₀) are those with formulae (F₈): 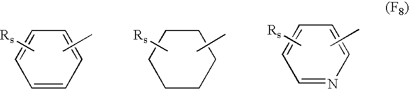
in which R₅ represents a hydrogen atom, an alkyl or alkoxy group, preferably C₁ to C₄, or an amino group which may or may not be substituted with an alkyl group, preferably C₁ to C₄.

Preferred bidentate ligands have formula (Ic₁) in which Φ represents a covalent bond, a methylene or ethylene group, or a divalent cyclic group such as: 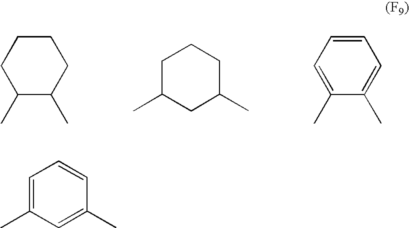
bidentate type ligands which are preferred in the process of the invention are those with formula (Ic₁) in which Φ represents a covalent bond, a methylene or ethylene group, one of groups (F₉) and Rₐ represents one of the groups with formula (F₈).

Ligands with formula (Ic₁) are produced by reacting:
a dicarbonyl compound with formula: 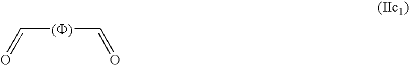
   in which formula (IIc₁), Φ has the meanings given in formula (Ic₁);
with a primary amine with formula (IIc₂)
   Rₐ—NH₂ (IIc₂)
   in which formula (IIc₂), Rₐ has the meanings given in formulae (Ia₁) or (Ia₂).

Preferred ligands with formula (Ic₂) used in the process of the invention contain two nitrogen atoms supplied by two imine functions. They preferably result from reacting an a or carbonylated compound, for example glyoxal, with an amine, preferably cyclohexylamine.

A preferred example of a ligand is given below:

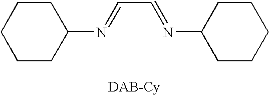

A further category of ligands which are suitable for carrying out the invention are the tridentate ligands: 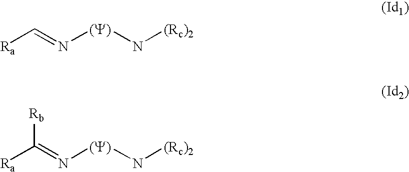
in which formulae:
   Rₐ, which may be identical or different, have the meanings given in formulae (Ia₁) and (Ia₂);
   R_{b}, which may be identical or different, have the meanings given in formulae (Ia₁) and (Ia₂);
   Rₐ and/or R_{b} may represent a hydrogen atom;
   R_{c}, which may be identical or different, have the meanings given in formulae (Ia₁) and (Ia₂); at most one of groups R_{c} represents a hydrogen atom;
   ψ represents a covalent bond or a skeleton with general formula (F₂) or (F₃(as defined in formulae (Ib₁) and (Ib₂).

In formulae (Id₁) and (Id₂) for tridentate type ligands, the preferred groups Rₐ and R_{b} preferably represent one of the groups with formula (F₀).

Preferred groups with formula (F₀) are those with formula (F₁₀): 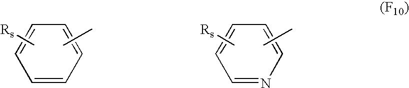
in which R₅ represents a hydrogen atom or an alkyl or alkoxy group, preferably C₁ to C₄, or an amino group which may or may not be substituted with alkyl groups, preferably C₁ to C₄.

In formulae (Id₁) and (Id₂) for tridentate type ligands, the group ψ is preferably a methylene or ethylene group.

Groups R_{c}, which may be identical or different, preferably represent an alkyl group containing 1 to 4 carbon atoms, preferably a methyl group.

Preferred tridentate type ligands are those with formula (Id₁).

Preferred tridentate type ligands for use in the process of the invention are those with formula (Id₁) in which groups R_{c}, which may be identical or different, represent an alkyl group containing 1 to 4 carbon atoms, preferably a methyl group, and Rₐ represents one of the following groups with formula (F₁₀) and the group y represents a methylene or ethylene group.

Tridentate ligands result from reacting:
an aldehyde or ketone with the following formulae: 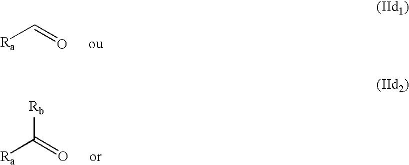
   in which formulae (Iid₁) and (Iid₂) Rₐ and R_{b} have the meanings given in formulae (Ia₁) or (Ia₂);
with a diamine with formula (Iid₃), preferably N,N-dimethylethylenediamine: 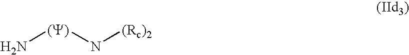
   in said formula (Iid₃), R_{c}, which may be identical or different, have the meanings given in formulae (Ia₁) or (Ia₂); at most one of groups R_{c} represent a hydrogen atom.

Preferred tridentate type ligands used in the process of the invention contain a nitrogen atom supplied by the pyridyl group of a pyridylaldehyde residue. Preferably, they result from reacting a pyridylaldehyde with a N-substituted or N,N-disubstituted diamine, preferably an alkyl group containing 1 to 4 carbon atoms.

A preferred example of a tridentate ligand is the following ligand (DAPAE):

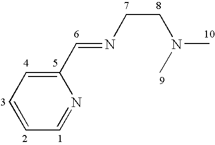

In accordance with the process of the invention, a nitrogen-containing type ligand is employed.

The ligands advantageously do not comprise atoms for chelating an oxygen atom or a group comprising an oxygen atom. However, the presence of an oxygen atom is possible in a functional group with not chelating function.

Preferred ligands from those cited above are: Chxn-Py-Al, Carbo-Py-Al, Py-Semizone, Chxn-Thio-Al, Py-Alzone, N-Amido-Py-Alzone and DAPAE.

It should be noted that the ligands used in the process of the invention can be employed in an optically pure form or in the form of a racemic mixture.

The ligands used in the process of the invention are known products.

The quantity in which they are used is a function of the quantity of the metallic copper element used.

It is generally such that the ratio between the number of moles of ligand and the number of moles of metal is in the range 20 to 0.9, preferably in the range 2 to 1.

It should be noted that the ligand can be introduced concomitantly with the compound supplying the catalytic metallic element. However, the invention also encompasses the case in which a metallic complex is prepared in advance by reacting the compound supplying the copper and the ligand, then isolating.

This complex can be prepared extemporaneously or in situ before or during the reaction, by separately adding the ligand and the compound supplying the copper at the beginning of the reaction.

The invention also pertains to copper complexes and its optically active forms obtained from a tetradentate ligand.

More precisely, the complex has the following formula:
CUL₄X (C)
in which formula:
   X represents a halogen atom;
   L₄ represents a ligand having formula (Ib₁) or (Ib₂) in which ψ has the meaning given in said formulae, R_{b} represents a hydrogen atom or a methyl group and Rₐ represents a pyridyl group with formula: 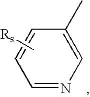
   in which R₅ has the meaning given above in formulae (F₀).

Preferred complexes with formula C are those in which:
L₄ represents a ligand having formula (Ib₁) in which ψ represents a urea group or one of groups (F₆) or (F₇) and Rₐ represents a pyridyl group as defined above, in which R₅ has the meaning given for formulae (F₅);
X represents a chlorine, bromine or iodine atom.

More particularly, the invention pertains to the following complex:

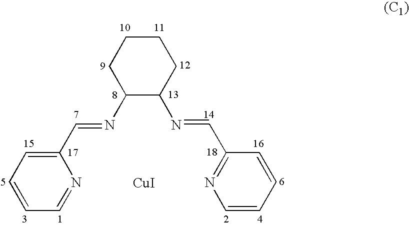

Complexes with formula C are preferably obtained by bringing the ligand, generally dissolved in a suitable solvent, for example of the ether type, preferably ethyl ether, into contact with a copper halide, also dissolved in an organic solvent, for example acetonitrile or any other solvent suitable to dissolve it.

After keeping the reaction medium stirred, usually at annular temperature (15° C. to 25° C.), the complex which precipitates out is separated using conventional solid/liquid separation techniques, for example by filtering.

The reaction of this liganded metallic complex can also catalyze the reactions of the invention, more particularly for the arylation reaction.

The process of the invention is of importance to a large number of nucleophilic compounds and examples are given below by way of illustration which are not limiting in any way.

A first category of substrates to which the process of the invention is applicable is formed by organic nitrogen-containing derivatives, more particular primary or secondary amines; hydrazine or hydrazone derivatives; amides; sulphonamides; urea derivatives or heterocyclic derivatives, preferably nitrogen-containing and/or sulphur-containing derivatives.

More precisely, the primary or secondary amines can be represented by general formula:
R₁R₂NH (IIIa)
in which formula (IIIa):
   R₁, R₂, which may be identical or different, represent a hydrogen atom or have the meanings given for Rₐ and R_{b} in formula (Ia₁) and (Ia₂);
   at most one of R₁ and R₂ represents a hydrogen atom.

Preferred amines have formula (IIIa) in which R₁, R₂, which may be identical or different, represent a C₁ to C₁₅ alkyl group, preferably C₁ to C₁₀, a C₃ to C₈ cycloalkyl group, preferably C₅ or C₆, or a C₆ to C₁₂ aryl or arylalkyl group.

More particular examples of groups R₁ and R₂ that can be mentioned are C₁ to C₄ alkyl groups, phenyl, naphthyl or benzyl groups.

More specific examples of amines with formula (IIIa) that can be mentioned are aniline, N-methylaniline, diphenylamine, benzylamine and dibenzylamine.

It should be noted that the amino group can be in the form of anions. The counter-ion is a metal cation, preferably an alkali metal cation, more preferably sodium or potassium. Examples of such compounds that can be cited are sodium or potassium amide.

Other nucleophilic compounds that can be used in the process of the invention are hydrazine derivatives with formulae (IIIb), (IIIc) or (IIId):
NH₂—NH—COOR₃ (IIIb)
NH₂—NH—COR₄ (IIIc)
NH₂—N═CR₅R₆ (IIId)
in which formulae (IIIb) to (IIId):
   R₃, R₄, R₅, R₆, which may be identical or different, have the meanings given for R₁ and R₂ in formula (IIIa).

Groups R₃, R₄, R₅, R₆ more particularly represent a C₁ to C₁₅ alkyl group, preferably C₁ to C₁₀, a C₃ to C₈ cycloalkyl group, preferably C₅ or C₆, or a C₆ to C₁₂ aryl or aryl alkyl group.

In formulae (IIIb) to (IIId), R₃ preferably represents a tertiobutyl group, R₄ represents a methyl or phenyl group and R₅, R₆ represent a phenyl group.

The invention also encompasses amide type compounds, more particularly with formula (IIIe):
R₇—NH—CO—R₈ (IIIe)

In which formula (IIIe), R₇ and R₈ have the meanings given for R₁ and R₂ in formula (IIIa).

Examples of compounds with formula (IIIe) that can be cited are oxazolidine-2-one, benzamide and acetamide.

The invention is also applicable to sulphonamide type compounds.

They can have the following formula:
R₉—SO₂—NH—R₁₀ (IIIf)

In which formula (IIIf), R₉ and R₁₀ have the meanings given for R₁ and R₂ in formula (IIIa).

An example of a compound with formula (IIIf) that can be cited is tosylhydrazide.

Other types of nucleophilic substrates that can be mentioned are urea derivatives such as guanidines which can be represented by formula (IIIg): 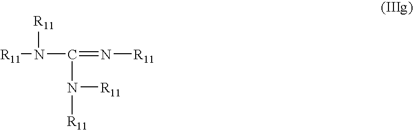
in which formula (IIIg), groups R₁₁, which may be identical or different, have the meanings given for R₁ and R₂ in formula (IIIa).

An example of a compound with formula (IIIg) that can be cited is N,N,N′,N′-tetramethylguanidine.

Nucleophilic substrates that are well suited to use in the process of the invention are heterocyclic derivatives comprising at least one nucleophilic atom such as a nitrogen, sulphur or phosphorus atom.

More precisely, they have general formula (IIIh): 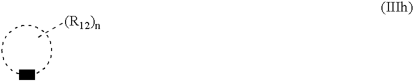
in which formula (IIIh):
   A represents the residue of a cycle forming all or a portion of a monocyclic or polycyclic, aromatic or non aromatic heterocyclic system wherein one of the carbon atoms is replaced by at least one nucleophilic atom such as a nitrogen, sulphur or phosphorus atom;
   R₁₂, which may be identical or different, represent substituents on the cycle;
   n represents the number of substituents on the cycle.

The invention is applicable to monocyclic heterocyclic compounds with formula (IIIh) in which A represents a saturated or non-saturated or aromatic heterocycle in particular containing 5 or 6 atoms in the cycle and possibly containing 1 or 3 heteroatoms such as nitrogen, sulphur or oxygen, at least one of which is a nucleophilic atom such as NH or S.

A can also represent a polycyclic heterocyclic compound defined as being constituted by at least 2 aromatic or non aromatic heterocycles containing at least one heteroatom in each cycle and forming ortho- or ortho- and pericondensed systems between them, or a group constituted by at least one aromatic or non aromatic carbocycle and at least one aromatic or non aromatic heterocycle forming ortho- or ortho- and peri-condensed systems between them.

It is also possible to start from a substrate resulting from a concatenation of a saturated, unsaturated or aromatic heterocycle as described above and a saturated, unsaturated or aromatic carbocycle. The term “carbocycle” preferably means a cycloaliphatic or aromatic cycle containing 3 to 8 carbon atoms, preferably 6.

It should be noted that the carbon atoms of the heterocycle can optionally be substituted with groups R₁₂, either completely or partially.

The number of substituents present on the cycle depends on the number of atoms in the cycle and on the presence or otherwise of unsaturated bonds on the cycle.

The maximum number of substituents that can be carried by the cycle can readily be determined by the skilled person.

In formula (IIIh), n is a number equal to 4 or less, preferably 0 or 1.

Examples of substituents are given below, but this list is not limiting in nature.

Group or groups R₁₂, which may be identical or different, preferably represent one of the following groups:
a linear or branched C₁ to C₆ alkyl group, preferably C₁ to C₄, such as methyl, ethyl, propyl, isopropyl, butyl, isobutyl, sec-butyl or tert-butyl;
a linear or branched C₂ to C₆ alkenyl or alkynyl group, preferably C₂ to C₄, such as vinyl or allyl;
a linear or branched C₁ to C₆ alkoxy or thioether group, preferably C₁ to C₄ such as methoxy, ethoxy, propoxy, isopropoxy or butoxy, or an alkenyloxy group, preferably an allyloxy or phenoxy group;
a cyclohexyl, phenyl or benzyl group;
a group or function such as: hydroxyl, thiol, carboxyl, ester, amide, formyl, acyl, aroyl, amide, urea, isocyanate, thioisocyanate, nitrile, nitride, nitro, sulphone, sulphonic, halogen, pseudohalogen or trifluoromethyl.

The present invention is particularly applicable to compounds with formula (IIIh) in which groups R₁₂ more particularly represent an alkyl or alkoxy group.

More particularly, optionally substituted residue A represents one of the following cycles:
a monocyclic heterocycle containing one or more heteroatoms: 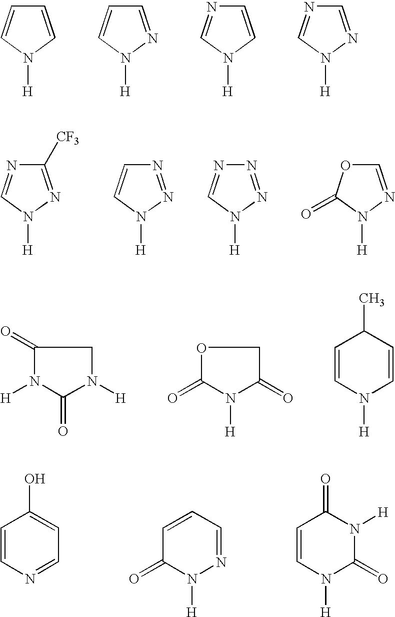
a bicycle comprising a carbocycle and a heterocycle comprising one or more heteroatoms; 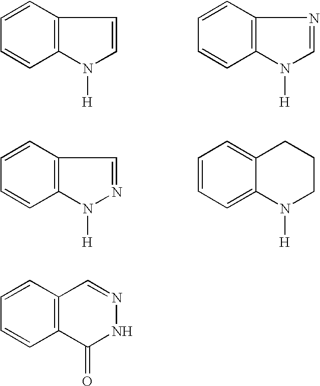
a tricycle comprising at least one carbocycle or a heterocycle comprising one or more heteroatoms;

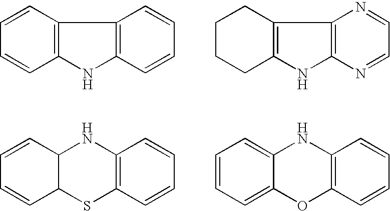

Preferred examples of heterocyclic compounds are those with formula (IIIh) in which A represents a cycle such as: imidazole, pyrazole, triazole, pyrazine, oxadiazole, oxazole, tetrazole, indole, pyrole, phthalazine, pyridazine or oxazolidine.

Nucleophilic compounds that can also be used in the process of the invention that can be cited are alcohol or thiol type compounds represented by the following formula:
R₁₃-Z (IIIi)
in which formula (IIIi):
   R₁₃ represents a hydrocarbon group containing 1 to 20 atoms and has the meanings given for R₁ or R₂ in formula (IIIa);
   Z represents a OM₁ or SM₁ type group in which M₁ represents a hydrogen atom or a metallic cation, preferably an alkali metal cation.

Preferred compounds have formula (IIIi) in which R₁₃ represents a hydrocarbon group containing 1 to 20 carbon atoms, which may be a linear or branched, saturated or unsaturated acyclic aliphatic group; a monocyclic or polycyclic, saturated, unsaturated or aromatic carbocyclic or heterocyclic group; or a concatenation of said groups.

More precisely, R₁₃ preferably represents a linear or branched saturated acyclic aliphatic group preferably containing 1 to 12 carbon atoms, more preferably 1 to 4 carbon atoms.

The invention also encompasses the presence of an unsaturated bond in the hydrocarbon chain such as one or more double bonds, which may or may not be conjugated, or a triple bond.

As mentioned for Rₐ defined in formula (Ia₁) or (Ia₂), the hydrocarbon chain can optionally be interrupted by a heteroatom or a functional group, or it may carry one or more substituents.

In formula (IIIi), R₁₃ can also represent a saturated or non saturated carbocyclic group, preferably containing 5 or 6 carbon atoms in the cycle; a saturated or non saturated heterocyclic group, containing 5 or 6 carbon atoms in the cycle including 1 or 2 heteroatoms such as nitrogen, sulphur, oxygen or phosphorus atoms; a monocyclic, aromatic heterocyclic carbocyclic group, preferably phenyl, pyridyl, furyl, pyrannyl, thiophenyl, thienyl, phospholyl, pyrazolyl, imidazolyl or pyrolyl, or a polycyclic, aromatic heterocyclic carbocyclic group which may or may not be condensed, preferably naphthyl.

When R₁₃ includes a cycle, it can also be substituted. The nature of the substituent is unimportant provided that it does not interfere with the principal reaction. The number of substituents is generally at most 4 per cycle, usually 1 or 2. Reference should be made to the definition of R₁₂ in formula (IIIh).

The invention also encompasses the case in which R₁₃ comprises a concatenation of aliphatic and/or cyclic, carbocyclic and/or heterocyclic groups.

One acyclic aliphatic group may be connected to a cycle via a covalent bond, a heteroatom or a functional group such as oxy, carbonyl, carboxy, sulphonyl, etc . . . .

More particular groups are cycloalkylalkyl, for example cyclohexylalkyl, or aralkyl groups containing 7 to 12 carbon atoms, in particular benzyl or phenylethyl.

The invention also encompasses a concatenation of carbocyclic and/or heterocyclic groups, more particularly a concatenation of phenyl groups separated by a covalent bond or an atom or a functional group G such as: oxygen, sulphur, sulpho, sulphonyl, carbonyl, carbonyloxy, imino, carbonylimino, hydrazo or alkylene (C₁-C₁₀, preferably C₁)-diimino.

The linear or branched, saturated or unsaturated acyclic aliphatic group can optionally carry a cyclic substituent. The term “cycle” means a saturated, unsaturated or aromatic carbocyclic or heterocyclic cycle.

Preferred compounds with formula (IIIi) have general formula (IIIi₁): 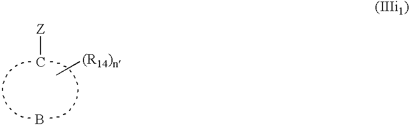
in which:
   B represents the residue of a monocyclic or polycyclic, aromatic, carbocyclic group or a divalent group constituted by a concatenation of two or more monocyclic aromatic carbocyclic groups;
   R₁₄ represents one or more substituents, which may be identical or different;
   Z represents an OM₁ or SM₁ group in which M₁ represents a hydrogen atom or a metallic cation, preferably an alkali metal cation;
   n′ is 5 or less.

Examples of substituents R₁₄ can be found by referring to those for R₁₂ defined for formula (IIIh).

More particular compounds with formula (IIIi₁) are those in which residue (B) represents:
a monocyclic or polycyclic aromatic carbocyclic group with cycles that can together form an ortho-condensed system with formula (F₁₁): 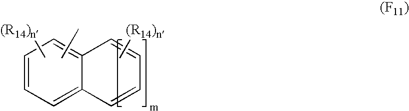
in which formula (F₁₁), m represents 0, 1 or 2 and symbols R₁₄ and n′, which may be identical or different, have the meanings given above;
a group constituted by a concatenation of two or more monocyclic aromatic carbocyclic groups with formula (F₁₂): 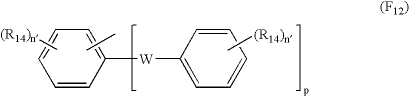
in which formula (F₁₂), symbols R₁₄ and n′, which may be identical or different, have the meanings given above, p is 0, 1, 2 or 3 and w represents a covalent bond, an alkylene or alkylidene C₁ to C₄ group, preferably a methylene group or isopropylidene group, or a functional group such as G.

Preferred compounds with formula (IIIi) have formulae (F₁₁) and (F₁₂) in which:
R₁₄ represents a hydrogen atom, a hydroxyl group, a —CHO group, a —NO₂ group, or a linear or branched alkyl or alkoxy group containing 1 to 6 carbon atoms, preferably 1 to 4 carbon atoms, more preferably methyl, ethyl, methoxy or ethoxy;
w represents a covalent bond, an alkylene or alkylidene group containing 1 to 4 carbon atoms or an oxygen atom;
m is 0 or 1;
n′ is 0, or 2;
p is 0 or 1.

Illustrative examples of compounds with formula (IIIi) that can in particular be mentioned are:
those in which residue B has formula (F₁₁) in which m and n′ equal 0, such as phenol or thiophenol;
those in which residue B has formula (F₁₁) in which m equals 0 and n′ equals 1, such as hydroquinone, pyrocatechine, resorcin, alkylphenols, alkylthiophenols, alkoxyphenols, salicylic aldehyde, p-hydroxybenzaldehyde, methyl salicylate, p-hydroxybenzoic acid methyl ester, chlorophenols, nitrophenols or p-acetamidophenol;
those in which residue B has formula (F₁₁) in which m equals 0 and n′ equals 2, such as dialkylphenols, vanillin, isovanillin, 2-hydroxy-5-acetamidobenzaldehyde, 2-hydroxy-5-propionamidobenzaldehyde, 4-Allyloxybenzaldehyde, dichlorophenols, methylhydroquinone or chlorohydroquinone;
those in which residue B has formula (F₁₁) in which m equals 0 and n′ equals 3, such as 4-bromovanillin, 4-hydroxyvanillin, trialkylphenols, 2,4,6-trinitrophenol, 2,6-dichloro-4-nitrophenol, trichlorophenols, dichlorohydroquinones or 3,5-dimethoxy-4-benzaldehyde;
those in which residue B has formula (F₁₁) in which m equals 1 and n′ is 1 or more, such as dihydroxynaphthalene, 4-methoxy-1-naphthol or 6-bromo-2-naphthol;
those in which residue B has formula (F₁₂) in which p is 1 and n′ is 1 or more, such as 2-phenoxyphenol, 3-phenoxyphenol, phenylhydroquinone, 4,4′-dihydroxybiphenyl, isopropylidene 4,4′-diphenol (bisphenol A), bis(4-hydroxyphenyl)methane, bis(4-hydroxyphenyl)sulphone, bis(4-hydroxyphenyl)sulphoxide or tetrabromo bisphenol A.

Other nucleophilic compounds that can be used in the process of the invention are hydrocarbon derivatives containing a nucleophilic carbon.

More particular examples are malonate type anions comprising a —OOC—HC⁻—COO— group.

Alkyl malonate or cyanomalonate anions with formula (IIIj1) or (IIIj2) can be mentioned:
R₁₅—OOC—C⁻(R₁₅″)—COO—R_{15′} (IIIj1)
R₁₅—OOC—C⁻(R₁₅″)—CN (IIIj2)
in which formula (IIIj1) and (IIIj2),
   R₁₅ and R₁₅′, which may be identical or different, represent an alkyl group containing 1 to 12 atoms in the alkyl group, preferably 1 to 4 atoms;
   R₁₅″ represents:
      a hydrogen atom;
      an alkyl group containing 1 to 12 carbon atoms;
      a cycloalkyl group containing 5 or 6 carbon atoms;
      a cycloalkyl group containing 5 or 6 carbon atoms, substituted with one or more alkyl radicals containing 1 to 4 carbon atoms, or alkoxy containing 1 to 4 carbon atoms;
      a phenyl group;
      a phenyl group substituted with one or more alkyl radicals containing 1 to 4 carbon atoms or alkoxy radicals containing 1 to 4 carbon atoms or by one or more halogen atoms;
      a phenylalkyl group the aliphatic portion of which contains 1 to 6 carbon atoms.

It is also possible to cite malodinitrile type anions containing a NC—C⁻(R₁₅″)—CN group in which R₁₅″ has the meaning given above.

It is also possible to use nitrile type compounds represented by formula (IIIk):
R₁₆—CN (IIIk)
in which formula R₁₆ has any nature and has the meanings given for R₁ and also represents a metallic cation, preferably an alkali cation, more preferably lithium, sodium or potassium.

R₁₆ has the meanings given for R₁.

Examples of nitrites that can be mentioned are acetonitrile, cyanobenzene optionally carrying one or more substituents on the benzene ring, or ethanal cyanhydrine CH₃CH(OH)CN.

It is also possible to use acetylenide type compounds in the process of the invention.

They can be represented by the formula (IIIm):
R₁₇—C≡⁻ (IIIm)
in which formula R₁₇ is of any nature and the counter-ion is a metal cation, preferably a sodium or potassium atom.

R₁₇ has the meanings given for R₁.

Particular examples that can be cited are sodium or potassium acetylide or diacetylide.

Other classes of nucleophilic compounds that can be employed in the process of the invention that can be cited are profene type compounds and their derivatives represented by the following formula:
R₁₈—HC⁻—COO—R₁₉ (IIIn)
in which formula:
   R₁₈ has the meanings given for R₁;
   R₁₉ represents an alkyl group containing 1 to 12 atoms in the alkyl group, preferably 1 to 4 atoms.

Preferred compounds are those with formula (IIIn) in which R₁₈ represents an alkyl group containing 1 to 12 carbon atoms, a cycloalkyl group containing 5 or 6 carbon atoms and an aryl group containing 6 or 12 carbon atoms or a nitrogen-containing heterocycle containing 5 or 6 atoms.

A further category of nucleophiles that can be used in the process of the invention is formed by amino acids and their derivatives: 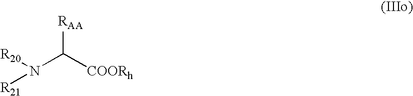
in which formula:
   R_{AA} represents the residue of an amino acid, preferably a hydrogen atom, a linear or branched C₁ to C₁₂ alkyl group optionally carrying a functional group, an aryl group or an arylalkyl C₆ to C₁₂ group or a functional group, preferably a hydroxyl group;
   R₂₀ and R₂₁ have the meanings given for R₁ and R₂ in formula (IIIa);
   Rₕ represents a hydrogen atom, a metal cation, preferably an alkali metal cation or a hydrocarbon group containing 1 to 12 carbon atoms, preferably a C₁ to C₁₂ alkyl group.

In formula (IIIo), R_{AA} represents an alkyl group that can carry a functional group, examples of which that can be cited being an —OH, —NH₂, —CO—NH₂, —NH—CNH—, —HN—C(O)—NH₂—, —COOH, —SH, —S—CH₃ group or an imidazole, pyrole or pyrazole group.

Examples of amino acids that can be cited are glycine, cysteine, aspartic acid, glutamic acid and histidine.

Examples of nucleophilic compounds which can be mentioned are those comprising a carbanion the counterion of which is a metal and having the following formulae: 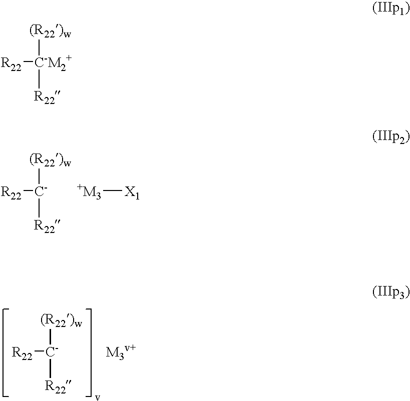
in which:
   group R₂₂ represents:
      an alkyl group containing 1 to 12 carbon atoms;
      a cycloalkyl group containing 5 or 6 carbon atoms;
      a cycloalkyl group containing 5 or 6 carbon atoms, substituted with one or more alkyl radicals containing 1 to 4 carbon atoms, or alkoxy radicals containing 1 or 4 carbon atoms;
      a phenylalkyl group the aliphatic portion of which comprises 1 to 6 carbon atoms;
      a phenyl group;
      a phenyl group substituted with one or more alkyl radicals containing 1 to 4 carbon atoms or alkoxy radicals containing 1 to 4 carbon atoms or by one or more halogen atoms;
      a saturated, unsaturated or aromatic heterocyclic group, preferably containing 5 or 6 atoms and comprising sulfur, oxygen or nitrogen as the heteroatom;
groups R₂₂′ and R₂₂″ represent a hydrogen atom or a group R₂₂;
two of groups R₂₂, R₂₂′ and R₂₂″ can be connected together to form a saturated, unsaturated or aromatic carbocycle or heterocyle preferably containing 5 or 6 carbon atoms;
M₂ represents a metallic element from group (IA) of the periodic table;
M₃ represents a metallic element from groups (IIA), (IIB) of the periodic table;
X₁ represents a chlorine or bromine atom;
v is the valency of metal M₃;
   w is 0 or 1.

Reference in the present text to the periodic table is to the periodic table published in the Bulletin de la Société Chimique de France, no 1 (1966).

Preferred compounds with formula (IIIp1) to (IIIp3) are those with lithium, sodium, magnesium or zinc as the metal and X₁ represents a chlorine atom.

Groups R₂₂, R₂₂′ and R₂₂″ are advantageously a C₁-C₄ alkyl group, a cyclohexyl or phenyl group; or said groups can form a benzene, cyclopentadienyl, pyridinyl or thiphenyl ring.

Examples that can be cited are n-butyllithium, t-butyl lithium, phenyl lithium, methyl or ethyl- or phenyl magnesium chloride, diphenyl magnesium, dimethyl or diethyl zinc, ccyclopentadiene zinc, and ethyl zinc chloride or bromide.

Examples of nucleophilic compounds of any other nature that can be mentioned are phosphorus or phosphorus- and nitrogen-containing compounds, more particularly those with the following formulae:
phosphides with formula (R₂₃)₂—P⁻ (IIIq)
phosphines with formula (R₂₃)₃—P (IIIr)
phosphonium diazoylides with formula (R₂₃)₃—P⁺—N²⁻ (IIIs)
phosphonium azoylides with formula (R₂₃)₃—P⁺—N⁻R₂₄ (IIIt)
in which formulae (IIIq) to (IIIt), groups R₂₃, which may be identical or different, and group R₂₄ represent:
   an alkyl group containing 1 to 12 carbon atoms;
   a cycloalkyl group containing 5 or 6 carbon atoms;
   a cycloalkyl group containing 5 or 6 carbon atoms, substituted with one or more alkyl radicals containing 1 to 4 carbon atoms, or alkoxy radicals containing 1 or 4 carbon atoms;
   a phenylalkyl group the aliphatic portion of which contains 1 to 6 carbon atoms;
   a phenyl group;
   a phenyl group substituted with one or more alkyl radicals containing 1 to 4 carbon atoms or alkoxy containing 1 to 4 carbon atoms or one or more halogen atoms.

More particular examples of phosphorus-containing compounds that can be cited are tricyclohexylphosphine, trimethylphosphine, triethylphosphine, tri-n-butylphosphine, triisobutylphosphine, tri-tert-butylphosphine, tribenzylphosphine, dicyclohexylphenylphosphine, triphenylphosphine, dimethylphenylphosphine, diethylphenylphosphine and di-tert-butylphenylphosphine.

Other nucleophilic compounds that can be used include boronic acids or their derivatives, more particularly those with the following formula: 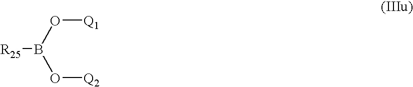
in which:
   R₂₅ represents a monocyclic or polycyclic, aromatic, carbocyclic or heterocyclic group;
   Q₁, Q₂, which may be identical or different, represent a hydrogen atom, a linear or branched, saturated or unsaturated aliphatic group containing 1 to 20 carbon atoms, or a R₂₅ group.

More precisely, the boronic acid has formula (IIIu) in which group R₂₅ represents an aromatic carbocyclic or heterocylic group. R₂₅ can have the meanings given above for B in formula (IIIi₁). However, R₂₅ more particularly represents a carbocyclic group such as a phenyl, naphthyl or heterocyclic group such as a pyrrolyl, pyridyl, pyrimidyl, pyridazinyl, pyrazinyl, 1,3-thiazolyl, 1,3,4-thiadiazolyl or a thienyl group.

The aromatic cycle can also be substituted. The number of substituents is generally at most 4 per cycle, but usually it is 1 or 2. Reference should be made to the definition of R₁₂ in formula (IIIh) for examples of substituents.

Preferred substituents are alkyl or alkoxy groups containing 1 to 4 carbon atoms, an amino group, a nitro group, a cyano group, a halogen atom or a trifluoromethyl group.

Q₁, Q₂, which may be identical or different, more particularly represent a hydrogen atom, or a linear or branched acyclic aliphatic group containing 1 to 20 carbon atoms which may be saturated or contain one or more unsaturated bonds in the chain, preferably 1 to 3 unsaturated bonds, preferably simple or conjugated double bonds.

Q₁, Q₂, preferably represent an alkyl group containing 1 to 10 carbon atoms, preferably 1 to 4, or an alkenyl group containing 2 to 10 carbon atoms, preferably a vinyl or a 1-methylvinyl group.

Q₁, Q₂, can have the meanings given for R₂₅; in particular, any cycle can also carry a substituent as described above.

Preferably, R₂₅ represents a phenyl group.

The scope of the present invention encompasses derivatives of boronic acids such as anhydrides and esters, more particularly alkyl esters containing 1 to 4 carbon atoms.

Particular examples of arylboronic acids that can be cited are: benzeneboronic acid, 2-thiopheneboronic acid; 3-thiopheneboronic acid; 4-methylbenzeneboronic acid, 3-methylthiophene-2-boronic acid, 3-aminobenzeneboronic acid, 3-aminobenzeneboronic acid hemisulphate, 3-fluorobenzeneboronic acid, 4-fluorobenzeneboronic acid, 2-formylbenzeneboronic acid, 3-formylbenzeneboronic acid, 4-formylbenzeneboronic acid, 2-methoxybenzeneboronic acid, 3-methoxybenzeneboronic acid, 4-methoxybenzeneboronic acid, 4-chlorobenzeneboronic acid, 5-chlorothiophene-2-boronic acid, benzo[b]furan-2-boronic acid, 4-carboxybenzeneboronic acid, 2,4,6-trimethylbenzeneboronic acid, 3-nitrobenzeneboronic acid, 4-(methylthio)benzeneboronic acid, 1-naphthaleneboronic acid, 2-naphthaleneboronic acid, 2-methoxy-1-naphthaleneboronic acid, 3-chloro-4-fluorobenzeneboronic acid, 3-acetamidobenzeneboronic acid, 3-trifluoromethylbenzeneboronic acid, 4-trifluoromethylbenzeneboronic acid, 2,4-dichlorobenzeneboronic acid, 3,5-dichlorobenzeneboronic acid, 3,5-bis(trifluoromethyl)benzeneboronic acid, 4,4′-biphenyldiboronic acid, and esters and anhydrides of said acids.

The present text provides lists of nucleophilic compounds that are in no way limiting and any type of nucleophilic compound can be envisaged.

In accordance with the process of the invention, a —C—C or —C-Nu-(O,S,P,N,Si, B . . . ) bond can be created by reacting a nucleophilic compound with a compound comprising an unsaturated bond in the position a to a leaving group.

More precisely, it is a compound comprising a leaving group Y represented by the formula (IV):
R₀—Y (IV)
in which formula R₀ represents a hydrocarbon group containing 2 to 20 carbon atoms and has a double bond or a triple bond located in the position a to a leaving group Y, or a monocyclic or polycyclic, aromatic, carbocyclic and/or heterocyclic group.

In accordance with the process of the invention, the compound with formula (III) is reacted with a compound with formula (IV) in which:
R₀ represents an aliphatic hydrocarbon group containing a double bond or a triple bond in the position a to the leaving group or a cyclic hydrocarbon group containing an unsaturated bond carrying a leaving group;
R₀ represents a monocyclic or polycyclic, aromatic, carbocyclic and/or heterocyclic group;
Y represents a leaving group, preferably a halogen atom, or a sulphonic ester group with formula —OSO₂—Rₑ, in which Rₑ is a hydrocarbon group.

The compound with formula (IV) will henceforth be designated as a “compound carrying a leaving group”.

In the formula for the sulphonic ester group, Rₑ is a hydrocarbon group of any nature. However, given that Y is a leaving group, it is advantageous from an economic viewpoint for Rₑ to be simple in nature, and more particularly to represent a linear or branched alkyl group containing 1 to 4 carbon atoms, preferably a methyl or ethyl group, but it can also represent a phenyl or tolyl group or a trifluoromethyl group, for example. The preferred group Y is a triflate group, which corresponds to a group Rₑ representing a trifluoromethyl group.

Bromine or chlorine atoms constitute preferred leaving groups.

More particularly, compounds with formula (IV) used in accordance with the process of the invention can be classified into three groups:
(1) aliphatic type compounds, carrying a double bond which can be represented by formula (IVa): 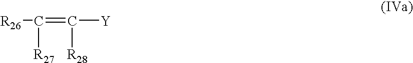
   in which formula (IVa):
      R₂₆, R₂₇ and R₂₈, which may be identical or different, represent a hydrogen atom or a hydrocarbon group containing 1 to 20 carbon atoms, which can be a linear or branched, saturated or unsaturated aliphatic group; a monocyclic or polycyclic, saturated, unsaturated or aromatic carbocyclic or heterocyclic group; or a concatenation of aliphatic and/or carbocyclic and/or heterocyclic groups as defined above;
      Y represents the leaving group, as defined above;
(2) aliphatic type compounds, carrying a triple bond, represented by formula (IVb):
   R₂₆—C═C—Y (IVb)
   in which formula (IVb):
      R₂₆ has the meaning given in formula (IVa);
      Y represents a leaving group as defined above;
(3) aromatic type compounds, hereinafter designated as a “halogenoaromatic compound” and which can be represented by formula (IVc): 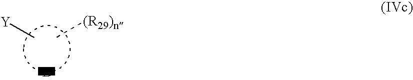
   in which:
      D represents the residue of a cycle forming all or a portion of a monocyclic or polycyclic, aromatic, carbocyclic and/or heterocyclic system;
      R₂₉, which may be identical or different, represent substituents on the cycle;
      Y represents a leaving group as defined above;
      n″ represents the number of substituents on the cycle.

The invention is applicable to unsaturated compounds with formulae (IVa) and (IVb) in which R₂₆ preferably represents a saturated linear or branched acyclic aliphatic group preferably containing 1 to 12 carbon atoms.

The invention does not exclude the presence of a further unsaturated bond on the hydrocarbon chain, such as a further triple bond or one or more double bonds, which may or may not be conjugated.

The hydrocarbon chain can optionally be interrupted by a heteroatom (for example oxygen or sulphur) or by a functional group provided that it does not react; in particular, a group such as —CO— can be cited.

The hydrocarbon chain can optionally carry one or more substituents provided that they do not react under the reaction conditions; particular mention can be made of a halogen atom, a nitrile group or a trifluoromethyl group.

The linear or branched, saturated or unsaturated acyclic aliphatic group can optionally carry a cyclic substituent. The term “cycle” means a saturated, unsaturated or aromatic, carbocyclic or heterocyclic cycle.

The acyclic aliphatic group can be connected to the cycle via a covalent bond, a heteroatom or a functional group such as oxy, carbonyl, carboxy, sulphonyl, etc . . .

Examples of cyclic substituents that can be envisaged are cycloaliphatic, aromatic or heterocyclic substituents, in particular cycloaliphatic, containing 6 carbon atoms in the cycle, or benzenic, said cyclic substituents themselves optionally carrying any substituent provided that they do not interfere with the reactions occurring in the process of the invention. Particular mention can be made of alkyl or alkoxy groups containing 1 to 4 carbon atoms.

More particular examples of aliphatic groups carrying a cyclic substituent are aralkyl groups containing 7 to 12 carbon atoms, in particular benzyl or phenylethyl.

In formulae (IVa) and (IVb), R₂₆ can also represent a carbocyclic group that may or may not be saturated, preferably containing 5 or 6 carbon atoms in the cycle, preferably cyclohexyl; a heterocyclic group, which may or may not be saturated, in particular containing 5 or 6 carbon atoms in the cycle 1 or 2 of which are heteroatoms such as nitrogen, sulphur or oxygen; a monocyclic aromatic carbocyclic group, preferably phenyl, or a polycyclic aromatic carbocyclic group, which may or may not be condensed, preferably naphthyl.

Regarding R₂₇ and R₂₈, they preferably represent a hydrogen atom or an alkyl group containing 1 to 12 carbon atoms, or a phenyl group or an aralkyl group containing 7 to 12 carbon atoms, preferably a benzyl group.

In formulae (IVa) and/or (IVb), R₂₆, R₂₇ and R₂₈ more particularly represent a hydrogen atom or R₂₆ represents a phenyl group and R₂₇, R₂₈ represent a hydrogen atom.

Examples of compounds with formulae (IVa) and (IVb) that can be cited are vinyl chloride or bromide, β-bromo- or β-chlorostyrene or bromoalkyne or iodoalkyne.

The invention is of particular application to halogenoaromatic compounds with formula (IVc) in which D is the residue of a cyclic compound, preferably containing at least 4 carbon atoms in its cycle, preferably 5 or 6, optionally substituted, and representing at least one of the following cycles:
a monocyclic or polycyclic aromatic carbocycle, i.e., a compound constituted by at least 2 aromatic carbocycles and between them forming ortho- or ortho- and peri-condensed systems, or a compound constituted by at least 2 carbocycles only one of which is aromatic and between them forming ortho- or ortho- and peri-condensed systems;
a monocyclic aromatic heterocycle containing at least one of heteroatoms P, O, N or S or a polycyclic aromatic heterocycle, i.e., a compound constituted by at least 2 heterocycles containing at least one heteroatom in each cycle wherein at least one of the two cycles is aromatic and between them forming ortho- or ortho- and peri-condensed systems, or a compound constituted by at least one carbocycle and at least one heterocycle at least one of the cycles being aromatic and forming ortho- or ortho- and peri-condensed systems between them.

More particularly, optionally substituted residue D preferably represents the residue of an aromatic carbocycle such as benzene, an aromatic bicycle containing two aromatic carbocycles such as naphthalene; or a partially aromatic bicycle containing two carbocycles one of which is aromatic, such as tetrahydro-1,2,3,4-naphthalene.

The invention also envisages the fact that D can represent the residue of a heterocycle provided that it is more electrophilic than the compound with formula (IIIh).

Particular examples that can be cited are an aromatic heterocycle such as furan or pyridine; an aromatic bicycle comprising an aromatic carbocycle and an aromatic heterocycle such as benzofuran or benzopyridine; a partially aromatic bicycle comprising an aromatic carbocycle and a heterocycle such as methylenedioxybenzene; an aromatic bicycle comprising two aromatic heterocycles such as 1,8-naphthylpyridine; a partially aromatic bicycle comprising a carbocycle and an aromatic heterocycle such as 5,6,7,8-tetrahydroquinoline.

In the process of the invention, a halogenoaromatic compound with formula (IVc) is preferably used in which D represents an aromatic nucleus, preferably a benzene or naphthalene nucleus.

The aromatic compound with formula (IVc) can carry one or more substituents.

In the present text, the term “several” generally means less than 4 substituents R₂₉ on the aromatic nucleus.

In formula (IVc), n″ is a number that is 4 or less, preferably 1 or 2.

Reference should be made to the definitions of R₁₂ in formula (IIIh) for examples of substituents.

R₂₉ also represents a saturated, unsaturated or aromatic heterocycle containing 5 or 6 atoms and comprising sulfur, oxygen or nitrogen as the heteroatom. Pyrazolyl or imidazolyl groups can also be mentioned.

In formula (IVc), n″ is a number less than or equal to 4, preferably 1 or 2.

Examples of compounds with formula (IVc) that can be cited are p-chlorotoluene, p-bromoanisole and p-bromotrifluorobenzene.

The quantity of compound carrying a leaving group with formula (IV), preferably with formula (IVa) or (IVb) or (IVc), is generally expressed with respect to the quantity of nucleophilic compound and is close to stoichiometry. The ratio between the number of moles of compound carrying a leaving group and the number of moles of nucleophilic compound is usually in the range 0.5 to 1.5, preferably in the range 0.9 to 1.2, and more preferably about 1.

In accordance with the process of the invention, the nucleophilic compound preferably with formulae (IIIa) to (IIIu) is reacted with a compound carrying a leaving group with formula (IV), preferably with formula (IVa) or (IVb) or (IVc), in the presence of an effective quantity of a catalyst based on copper and a ligand as defined in the invention.

Examples of catalysts that can be used that can be cited are copper metal or organic or inorganic compounds of copper (I) or copper (II).

The catalysts employed in the process of the invention are known products.

Examples of catalysts of the invention that can be cited are cuprous bromide, cupric bromide, cuprous iodide, cupric iodide, cupric chloride, basic copper (II) carbonate, cuprous nitrate, cupric nitrate, cuprous sulphate, cupric sulphate, cuprous sulphite, cuprous oxide, cuprous acetate, cupric acetate, cupric trifluoromethylsulphonate, cupric hydroxide, copper (I) methylate, copper (II) methyate and chlorocupric methylate with formula ClCuOCH₃.

Preferably, cuprous or cupric chloride or bromide or cuprous or cupric oxide are used.

The quantity of catalyst employed, expressed as the molar ratio between the number of moles of copper catalyst expressed as copper metal and the number of moles of compound with formula (IV) generally varies between 0.001 and 0.2, preferably between 0.01 and 0.1.

In a variation, the invention also encompasses the copper being associated with a small quantity of another metallic element designated M.

The metallic element M is selected from group (VIII), (IB) and (IIB) of the periodic table.

Examples of metals M that can be cited are silver, palladium, cobalt, nickel, iron and/or zinc.

Advantageously, a mixture comprising palladium and copper is used.

The palladium can be supplied in the form of a finely divided metal or in the form of an inorganic derivative such as an oxide or hydroxide. It is possible to use a mineral salt, preferably a nitrate, sulphate, oxysulphate, halide, oxyhalide, silicate, carbonate, or an organic derivative, preferably the cyanide, oxalate or acetylacetonate; an alcoholate, more preferably methylate or ethylate; or a carboxylate, still more preferably the acetate. It is also possible to use complexes, in particular chlorine-containing or cyanide containing complexes with palladium and/or alkali metals, preferably sodium, potassium or ammonium.

Examples of compounds that can be used to prepare the catalysts of the invention that can be cited are palladium (II) bromide, palladium (II) chloride, palladium (II) iodide, palladium (II) cyanide, hydrated palladium (II) nitrate, palladium (II) oxide, dihydrated palladium (II) sulphate, palladium (II) acetate, palladium (II) propionate, palladium (II) butyrate, palladium (II) benzoate, palladium (II) acetylacetonate, ammonium tetrachloropalladate (II), potassium hexachloropalladate (IV), palladium (II) tetramine nitrate, palladium (II) dichlorobis(acetonitrile), palladium (II) dichlorobis(benzonitrile), palladium (II) dichloro(1,5-cyclooctadiene), palladium (II) dichlorodiamine, palladium (0) tetrakistriphenylphosphine, palladium (II) acetate and trisbenzylideneacetone palladium (0).

Specific examples of nickel derivatives that can be cited are nickel (II) halides such as nickel (II) chloride, bromide or iodide; nickel (II) sulphate; nickel (II) carbonate; salts of organic acids containing 1 to 18 carbon atoms, in particular the acetate or propionate; nickel (II) complexes such as nickel (II) acetylacetonate, nickel (II) dibromo-bis-(triphenylphosphine), nickel (II) dibromo-bis(pyridine); or nickel (O) complexes such as nickel (O) bis-(cycloocta-1,5-diene) or nickel (O) bis-diphenylphosphinoethane.

It is also possible to use catalysts based on iron or zinc, generally in the form of the oxide, hydroxide or salts such as halides, preferably the chloride, nitrate or sulphate.

The quantity of metallic element M represents less than 50%, preferably less than 10 mole % of copper.

More preferably, a catalyst containing only copper is used.

A base, the function of which is to trap the leaving group, is also used in the process of the invention.

The feature of the base is that it has a pKa of 4 or more, preferably in the range 6 to 30.

The pKa is defined as the ionic dissociation constant of the acid/base pair when water is used as the solvent.

Reference should be made, inter alia, to the “Handbook of Chemistry and Physics”, 66^{th} edition, p. D-161 and D-162 in order to select a base with a suitable pKa.

Suitable bases that can be cited include mineral bases such as alkali metal carbonates, bicarbonates or hydroxides, preferably of sodium, potassium, caesium or alkaline-earth metals, preferably calcium, barium or magnesium.

It is also possible to use alkali metal hydrides, preferably sodium hydride or alkali metal alcoholates, preferably of sodium or potassium, more preferably sodium methylate, ethylate or tertiobutylate.

It is also possible to use organic bases as tertiary amines, more particularly triethylamine, tri-n-propylamine, tri-n-butylamine, methyldibutylamine, methyldicyclohexylamine, ethyldiisopropylamine, N,N-diethylcyclohexylamine, pyridine, dimethylamino-4-pyridine, N-methylpiperidine, N-ethylpiperidine, N-n-butylpiperidine, 1,2-methylpiperidine, N-methylpyrrolidine and 1,2-dimethylpyrrolidine.

Preferred bases are alkali metal carbonates.

The quantity of base employed is such that the ratio between the number of moles of base and the number of moles of aromatic compound carrying the leaving group is preferably in the range 1 to 4, preferably about 2.

The arylation or vinylation or alkynylation reaction of the invention is usually carried out in the presence of an organic solvent.

An organic solvent is used that does not react under the reaction conditions.

The type of solvent used is preferably a polar organic solvent, more preferably aprotic:
linear or cyclic carboxamides such as N,N-dimethylacetamide (DMAC), N,N-diethylacetamide, dimethylformamide (DMF), diethylformamide or 1-methyl-2-pyrrolidinone (NMP);
dimethylsulphoxide (DMSO);
hexamethylphosphotriamide (HMPT);
tetramethyurea;
nitro compounds such as nitromethane, nitroethane, 1-nitropropane, 2-nitropropane or mixtures thereof, and nitrobenzene;
aliphatic or aromatic nitriles such as acetonitrile, propionitrile, butanenitrile, isobutanenitrile, pentanenitrile, 2-methylglutaronitrile or adiponitrile;
tetramethylene sulphone (sulpholane);
organic carbonates such as dimethylcarbonate, diisopropylcarbonate or di-n-butylcarbonate;
alkyl esters such as ethyl or isopropyl acetate;
halogenated or non halogenated aromatic hydrocarbons such as chlorobenzene or toluene;
ketones, such as acetone, methylethylketone, methylisobutylketone, cyclopentanone, cyclohexanone;
nitrogen-containing heterocycles such as pyridine, picoline and quinolines.

It is also possible to use a mixture of solvents.

The quantity of organic solvent to be used is determined as a function of the nature of the selected organic solvent.

It is determined so that the concentration of the compound carrying a leaving group in the organic solvent is preferably in the range 5% to 40% by weight.

The arylation or vinylation or alkynylation reaction of the nucleophilic compound takes place at a temperature that is advantageously in the range 0° C. to 120° C., preferably in the range 20° C. to 100° C., more preferably in the range 25° C. to 80° C.

The arylation or vinylation or alkynylation reaction is generally carried out at atmospheric pressure, but higher pressures of up to 10 bars, for example, can also be used.

In practice, the reaction is simple to carry out.

The order of using the reagents is not critical. Preferably, the (preferably copper) catalyst, the ligand, the nucleophilic compound with formula (III), the base, the compound carrying a leaving group with formula (IV) and the organic solvent are charged.

The reaction medium is heated to the desired temperature.

The progress of the reaction is monitored by following the disappearance of the compound carrying a leaving group.

At the end of the reaction, a product of the type R-Nu-R₀ is obtained, more particularly an arylated compound comprising the residue of the nucleophilic compound and the residue of an electrophilic compound preferably with the following formula (V): 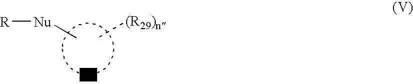
in which formula (V), D, R, R₂₉, Nu and n″ have the meanings given above.

The compound obtained is recovered using conventional techniques, in particular by crystallisation from an organic solvent.

More specific examples of organic solvents that can be mentioned are aliphatic or aromatic, halogenated or non halogenated hydrocarbons, carboxamides and nitrites. Particular mention can be made of cyclohexane, toluene, dimethylformamide and acetonitrile.

Examples of the invention will now be given. These examples are given by way of illustration and are not limiting in nature.

Before describing the examples, we shall describe the operating protocol used in all of the examples unless otherwise indicated. The preparation of certain ligands and catalysts is also illustrated.

In the examples, the degree of transformation (TT) corresponds to the ratio between the number of moles of substrate transformed and the number of moles of substrate engaged.

The yield (RR) corresponds to the ratio between the number of moles of product formed and the number of moles of substrate engaged.

The transformation yield (RT) or selectivity corresponds to the ratio between the number of moles of product formed and the number of moles of substrate engaged.

### EXAMPLES

### Operating Protocol

The following are successively introduced into a 35 ml Schlenk tube placed in a nitrogen atmosphere:
copper catalyst (0.05 mmoles);
ligand (0.1 mmoles);
nucleophilic compound (0.75 mmoles);
a base (1 mmoles);
56 μl of iodobenzene (0.5 mmoles);
and 300 μl of acetonitrile.

The mixture is placed in an oil bath at a temperature of 50° C. and stirred for 90 hours.

After this period, the mixture is diluted with ethyl ether or dichloromethane.

65 μl of internal reference (1,3-dimethoxybenzene) is introduced and a sample of reaction medium is removed then filtered over celite (or filter medium) eluting with ethyl ether or dichloromethane depending on the solubility.

The arylated compound obtained is extracted with ethyl ether or dichloromethane, then with distilled water and the product obtained is analysed by gas chromatography using 1,3-dimethoxybenzene as an internal reference.

Preparation of Ligands:

### a Preparation of trans-1,2-bis(2′-pyridylidenamino)-cyclohexane (Chxn-Py-Al) with formula

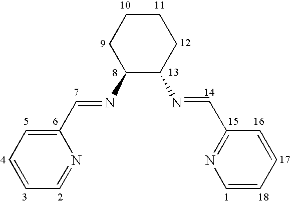

The ligand was prepared using the method described by Gao. H-X; Zhang, H.; Yi, X-D; Xu, P.-P.; Tang, C.-L.; Wan, H.-L.; Tsai, K.-R.; Ikariya, T.; (Chirality 2000, 12, 383-388).

12.65 g of anhydrous magnesium sulphate (105.1 mmoles) and 4.2 ml of a racemic trans-1,2-diaminocyclohexane mixture (35.0 mmoles) were successively added to a solution of 6.66 ml of 2-pyridylaldehyde (70.0 mmoles) in 50 ml of absolute ethanol.

The reaction mixture was stirred for 20 hours at ambient temperature (the solution turned yellow after stirring for three hours), heated for 2.5 hours under reflux, then filtered through a frit.

The isolated solid was washed with dichloromethane.

The total filtrate was concentrated completely under reduced pressure to isolate an ochre solid, which was re-crystallised from ethanol.

8.2 g of pale yellow crystals were obtained, which corresponded to a 80.1% yield.

The characteristics were as follows:

M.Pt: 140-141° C. (EtOH) (racemic mixture) (Lit: 127-129° C.: obtained by Belokon, Y N; North, M: Churkina, T D; Ikonnikov, N S; Maleev, V I; Tetrahedron 2001, 57, 2491-2498 for the stereoisomer 1S,2S, hexane-MeOH);

¹H NMR/CDCl₃: δ 8.51 (m, 2H, H_{1,2}), 8.28 (s, 2H, H_{7,14}), 7.84 (m, 2H, H_{4,17}), 6.55-7.64 (m, 2H, H_{5,16}), 7.14-7.21 (m, 2H, H_{3,18}), 3.50 (m, 2H, H_{8,13}), 1,81 (m, 6H, H_{10,11} and H carried by carbons 9 and 12 located in the position cis (or trans) with respect to the adjacent nitrogen atoms), 1.40-1.53 (m, 2H, H carried by carbons 9 and 12 located in the trans (or cis) position with respect to the adjacent nitrogen atoms).

¹³C NMR/CDCl₃: δ 161.42 (C7 and C14), 154.61 (C6 to C15), 149.21 (C1 and C2), 136.39 (C4 and C17), 124.43 (C3 and C18), 121.29 (C5 and C16), 73.53 (C8 and C13), 32.70 (C9 and C12), 24.33 (C10 and C11).

FAB+ (NBA matrix): 293 (100%, M+1), 107 (52%, 2-pyridylaldimine+H⁺), 92 (38%, C₅H₄N—CH₂⁺), 119 (25%, C₅H₄N—CH═N—CH₂⁺), 294 (23%, M+2), 204 (22%, [M-(2-pyridylidene)]⁺), 79 (21%, pyridine⁺), 187 (20%, M-[2-pyridylineamino]⁺), 585 (1%, 2M+1).

### b. Preparation of bis-(2-pyridylidene)-carbohydrazide (Carbo-Py-Al) with formula

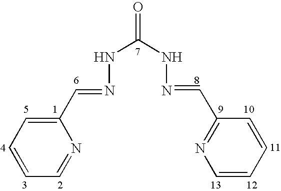

The ligand was prepared using the method described by Exner O; Kliegrnan, J M; J. Org. Chem. 1971, 36, 2014-2015.

8.96 g of anhydrous sodium sulphate (63.1 mmoles) and 4.0 ml of 2-pyridylaldehyde (42.05 mmoles) were added in succession to a suspension of 1.89 g of carbohydrazide (21.0 mmoles) in 150 ml of absolute ethanol.

The reaction mixture was heated for 4 hours under reflux then filtered through a frit (the disappearance of the 2-pyridylaldehyde was monitored by gas chromatography).

The retained solid was washed with copious amounts of absolute ethanol to dissolve the product obtained.

The filtrate was concentrated to isolate a colourless solid, which was oven dried at 100° C. then re-crystallised from methanol.

4.53 g of colourless crystals were obtained, corresponding to a yield of 80.5%.

The characteristics were as follows:

M.Pt: 219-220° C.;

¹H NMR/DMSO-d₆: δ 11.08 (wide s, 2H, NH), 8.58 (m, 2H, H_{2,13}), 8.25 (wide s, 2H, H_{6,8}), 8.12 (m, 2H, H_{5,10}), 7.87 (m, 2H, H_{4,11}), 7.38 (m, 2H, H_{3,12});

¹³C NMR/DMSO-d₆: δ 153.46 (C7), 151.64 (C1 and C19), 149.26 (C2 and C13), 143.69 (C6 and C8), 136.52 (C4 and C₁₁), 123.83 (C3 and C12), 119.75 (C5 and C₁₀);

FAB+ (NBA matrix): 269 (60%, M+1), 148 (51%, [C₅H₄NCH═N—NHCO]⁺), 122 (44%, C₅H₄N—CH═N—NH₃⁺), 107 (41%, 2-pyridylaldimine+H⁺), 537 (4%, 2M+1), 559 (1%, 2M+Na⁺).

### c. Preparation of 2-pyridylaldehyde N-methylhydrazone (Py-Alzone) with formula

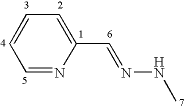

The ligand was prepared using the method described by Exner O; Kliegman, J M; J. Org. Chem. 1971, 36, 2014-2015.

8.96 g of anhydrous sodium sulphate (63.07 mmoles) and 2.24 ml of N-methylhydrazine (42.05 mmoles) were added in succession to a solution of 2.0 g of 2-pyridylaldehyde (21.02 mmoles) in 50 ml of absolute ethanol.

The reaction mixture was heated for 30 minutes at ambient temperature, then heated for 20 hours under refulx, then filtered through a frit.

The isolated sodium sulphate was washed with diethyl ether.

The total filtrate was concentrated completely under reduced pressure.

The orange oil obtained underwent the usual treatment (extraction with diethyl ether/water).

After drying over magnesium sulphate, filtering and concentration under reduced pressure, the yellow oil obtained was re-crystallised from methanol.

The crystals obtained were washed with copious quantities of petroleum ether to render them colourless.

1.4 g of crystals were obtained, corresponding to a yield of 49%.

The compound was relatively unstable and had to be prepared just prior to use.

The characteristics were as follows:

M.Pt: 44-45° C. (Lit: 39-40° C., obtained by Renwick, G M; Aust. J. Chem. 1970, 23, 2109-2117);

¹H NMR/CDCl₃: δ 8.50 (m, 1H, H₅), 7.71-7.76 (m, 1H, H₂), 7.57-7.66 (m, 1H, H₃), 7.55 (s, 1H, H₆), 7.07-7.14 (m, 1H, H₄), 5.92 (wide s, 1H, NH), 3.00 (s, 3H, H₇);

¹³C NMR/CDCl₃: δ 155.44 (C1), 149.09 (C5), 136.21 (C3), 134.10 (C6), 121.92 (C4), 119.04 (C2), 34.07 (C7);

GC/MS: Rt=13.75 min, M/Z=135, purity=100%.

### d Preparation of 2-pyridylaldehyde semicarbazone (N-amido-Py-Alzone) with formula

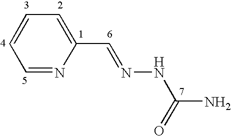

7.35 ml of triethylamine (52 mmoles) was added to a suspension of 5.8 g of semicarbazide hydrochloride (52 mmoles) in 60 ml of absolute ethanol.

The solution was heated to 50° C. then 5 ml of 2-pyridylaldehyde was rapidly added.

The mixture was heated under reflux for two hours, cooled to 20° C. then filtered through a frit.

The isolated yellow solid was washed with copious amounts of water, oven dried at 100° C. then re-crystallised from ethanol.

2.6 g of colourless crystals were obtained, which corresponded to a yield of 30%.

M.Pt: 204-206° C. (Lit: 206° C., EtOH obtained by Case, F H; Schilt, A A, J. Chem. Eng. Data 1980, 25, 404-405);

¹H NMR/DMSO-d₆: δ 10.56 (wide s, 1H, NH), 8.51 (m, 1H, H₅), 8.13 (m, 1H, H₂), 7.90 (s, 1H, H₆), 7.77 (m, 1H, H₃), 7.30 (m, 1H, H₄), 6.68 (wide s, 2H, NH₂);

¹³C NMR/DMSO-d₆: δ 156.57 (C7), 153.66 (C1), 149.03 (C5), 139.85 (C3), 136.32 (C6), 123.39 (C4), 119.50 (C2).

### e—Preparation of trans-1,2-bis(2′-thienylideneamino)-cyclohexane (Chxn-Tho-Al) with formula

This ligand has been described by Van Stein, G C; Van Loten, G; Vrieze, K, Inorg. Chem 1985, 24 (9), 1367-1375.

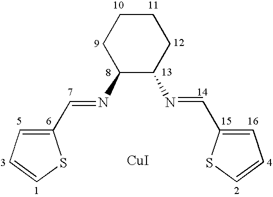

19.36 g of anhydrous magnesium sulphate (161.1 mmoles) and 6.44 ml of rac-trans-1,2-diaminocyclohexane (53.6 mmoles) were successively added to a solution of 10 ml of 2-thienylaldehyde (107.1 mmoles) in 75 ml of absolute ethanol.

The reaction mixture was stirred for 16 hours at ambient temperature (the solution thickened very rapidly), heated for 2 hours under reflux then filtered through a frit.

The isolated solid was washed with dichloromethane.

The total filtrate was concentrated completely under reduced pressure to isolate a brown solid which was re-crystallised from ethanol.

14.0 g of beige crystals were obtained, corresponding to a yield of 86%.

The characteristics were as follows:

M.Pt: 173-175° C. (EtOH);

¹H NMR/CDCl₃: δ 8.27 (s, 2H, H_{7,14}), 7.27 (m, 2H, H_{1,2}), 7.14 (m, 2H, H_{5,16}), 6.96 (m, 2H, H_{3,4}), 3.32 (m, 2H, H_{8,13}), 1.82 (m, 6H, H_{10,11} and H carried by carbons 9 and 12 located in the position cis (or trans) with respect to the adjacent nitrogen atoms), 1.44 (m, 2H, H carried by carbons 9 and 12 located in the trans (or cis) position with respect to the adjacent nitrogen atoms).

¹³C NMR/CDCl₃: δ 154.32 (C7 and C14), 142.54 (C6 to C15), 130.09 (C1 and C2), 128.20 (C5 and C16), 127.18 (C3 and C4), 73.38 (C8 and C13), 32.83 (C9 and C12), 24.44 (C10 and C11).

### f—Preparation of glyoxal dicyclohexylimine (DAB-Cy) with formula

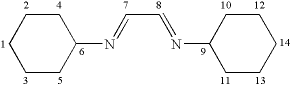

A mixture composed of 6.53 g of an aqueous solution of 40% by weight glyoxal (45.0 mmoles of glyoxal), 7 ml of n-propanol and 20 ml of water was added to a solution of 10 g of cyclohexylamine (100.8 mmoles) in 70 ml of n-propanol.

After heating for one and a half hours at 70° C., the mixture was cooled to ambient temperature.

Adding 100 ml of ice water caused precipitation of a large amount of white solid.

It was isolated by filtering through a frit, washed with water (3×50 ml) and methanol (1×25 ml) then vacuum dried.

8.5 g of product was obtained, corresponding to a yield of 86%.

The characteristics were as follows:

M.Pt: 144-145° C. (Literature: 145-147° C., obtained by Exner O; Kliegman, J M; J. Org. Chem. 1971, 36, 2014-2015);

¹H NMR/CDCl₃: δ 7.92 (s, 2H, H_{7,8}), 3.14 (m, 2H, H_{6,9}), 1,17-1.82 (m, 20H, H_{1,2,3,4,5,10,11,12,13,14});

¹³C NMR/CDCl₃: δ 160.10 (C7 and C8), 69.39 (C6 to C9), 33.95 (C4, C5, C10 and C11), 25.50 (C1 and C14), 24.57 (C2, C3, C12 and C13).

### g—Preparation of 1-(dimethylamino)-2-(2′-pyridylideneamino)-ethane with formula

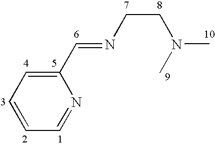

3.6 g of anhydrous magnesium sulphate (30.0 mmoles) and 2.15 ml of N,N-dimethylethylenediamine (20.0 mmoles) were successively added to a solution of 1.90 ml of 2-pyridylaldehyde (20.0 mmoles) in 18 ml of absolute ethanol.

The reaction mixture was stirred for 72 hours at ambient temperature then filtered through a frit.

The isolated solid was washed with dichloromethane.

The total filtrate was concentrated completely under reduced pressure to isolate a brown oil.

2.8 g of ligand were obtained, corresponding to a yield of 78%.

The characteristics were as follows:

¹H NMR/CDCl₃: δ 8.54 (ddd, 1H, ³J_{HH}=4.9 Hz, ⁴J_{HH}=1.7 Hz, ⁵J_{HH}=1.0 Hz, H₁), 8.35 (apparent s, 1H, H₆), 7.92 (ddd, H, ³J_{HH}=8.0 Hz, ⁴J_{HH}=1.2 Hz, ⁵J_{HH}=1.0 Hz, H₄), 7.92 (dddd, H, ³J_{HH}=8.0 Hz, ³J_{HH}=7.6 Hz, ⁴J_{HH}=1.7 Hz, ⁵J_{HH}=0.6 Hz, H₃), 7.25 (ddd, 2H, ³J_{HH}=7.6 Hz, ³J_{HH}=4.9 Hz, ⁴J_{HH}=1.2 Hz, H₂), 3.74 (td, 2H, H₇), 2.61 (t, 2H, H₈), 2.36 (s, 6H, H_{8.9})

GC/MS: rt=16.44 min, M/Z=177

Preparation of Catalysts:

The catalysts used were commercially available products with the exception of activated Cu(A) and activated Cu(B). An operating mode is also provided for preparing said catalysts, which were then used in the examples.

a—Activated Cu (A) prepared by purification of metallic copper:

A few grams of copper powder were ground for 15 minutes in a solution composed of 2 g of iodine dissolved in 100 ml of acetone.

The mixture was filtered through a frit, washed with 150 ml of a solution composed of concentrated hydrochloric acid (75 ml) and acetone (75 ml), using 100 ml of acetonitrile then 100 ml of acetone.

Elimination of all of the cuprous iodide was ensured by washing with acetonitrile, a solvent in which it is highly soluble (27.51 g/l).

The activated copper was dried in a vacuum dessiccator in the presence of P₂O₅.

It was used immediately after its preparation.

b—Activated Cu(B) Prepared by Reduction of Copper Sulphate

30 g of copper sulphate pentahydrate (120 mmoles) was dissolved in a solution composed of 100 ml of distilled water and 5 ml of hydrochloric acid.

1.96 g of zinc (30 mmoles) was slowly added to this solution, taking care that the temperature did not exceed 40° C.

The precipitated copper was isolated by filtering through a frit, washed with distilled water then with acetone and dried in a desiccator in the presence of P₂O₅.

It was used after preparation.

Preparation of CuI/Chxn-Py-Al Metal Complex

A solution of 652 mg of cuprous iodide (3.42 mmoles) dissolved in a minimum volume of acetonitrile was added, with stirring, to a solution of 1 g of trans-1,2-bis(2′-pyridylideneamino)cyclohexane (3.42 mmole) dissolved in a minimum volume of diethyl ether.

The mixture was stirred at ambient temperature for two hours then filtered through a frit to isolate a black powder, which was washed with acetonitrile and petroleum ether than dried in a dessicator under vacuum in the presence of phosphoric anhydride.

1.65 g of a black powder was obtained, which corresponded to a yield of 100% and to a purity of more than 98% as adjudged by ¹H NMR.

The metallic complex had the following formula:

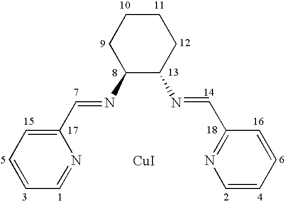

M Pt: 256° C. (MeOH/Et₂O) (compound not described in the literature).

¹H NMR/DMSO-d₆: δ 8.82 (broad sulfur, 2H), 8.28 (broad s, 2HO, 8.03 (broad m, 2H), 7.78 (broad s, 2H), 7.60 (broad s, 2H), 3.93 (m, 2H, H_{8,13}), 1.26-2.01 (m, 8H, Hg 12).

¹³C NMR/DMSO-d₆: δ 164.73 (C_{7, 14}), 161.43 (C_{1,2}), 151.07 (C_{17,18}), 148.63 (C_{5,6}), 137.98 (C_{3,4}), 127.31 (C_{15,16}), 70.46 (C_{8,13}), 32.86 (C_{9,12}), 23.75 (C_{10,11})

IR (KBr): v (cm⁻¹)=2935 and 2358 (w), 2192 (w), 1593 (S), 1471 and 1439 (w), 1384 (VS), 1291, 1223 and 1156 (w), 771 (VS), 746 (w)

FAB+ (NBA matrix): M/Z 545 (100%, ligand+⁶³Cu⁺+⁶³Cu⁺+I⁻, 547 (92%, ligand+⁶³Cu⁺+⁶⁵Cu⁺+I⁻), 355 (91% ligand+⁶³Cu⁺, 357 (40%, ligand+⁶³Cu⁺), 710 (33%, 2 ligands+2⁶³Cu⁺), 712 (31%, 2 ligands+⁶³Cu⁺+⁶⁵Cu⁺), 435 (28%), 437 (23%), 572 (20%), 460 (18%), 419 (14%), 249 (10%), 837 (10%, 2 ligands+2⁶³Cu⁺+I—), 839 (9%, 2 ligands+⁶³Cu⁺+⁶⁵Cu⁺+I⁻)

HRMS: calculated for C₁₈H₂₀N₄⁶³Cu(M-I—): 355.0984. found: 355.0986

Elemental analysis: calculated: C, 44.78; H 4.18; N 11.60; Cu 13.16. found: c 43.39; H, 4.15; N 11.34; Cu 12.77.

UV (MeOH): λ max=280 mm.

### Example 1

### N-Arylation and N-Vinylation of Azoles

Several operating protocols will now be given; their choice depends on the physical form of the nucleophile and the arylation agent.

Operating Protocol A: Solid Nucleophile and Liquid Arylation Agent

14.4 mg of cuprous oxide (0.1 mmoles), 116.8 mg of Chxn-Py-Al or another ligand as generally defined in this patent (0.4 mmoles), 3 mmoles of a nucleophilic compound and 1.303 g of caesium carbonate (4 mmoles) are successively introduced into a 35 ml Schlenk tube that has been oven dried at 100° C. and is provided with a magnetic stirrer (12×4.5 mm) and under a nitrogen atmosphere.

The Schlenk tube is purged under vacuum then refilled with nitrogen.

2 mmoles of arylation agent then 1.2 ml of acetonitrile or DMF are then added using syringes.

The reactor is placed in an oil bath at a temperature of 82° C. and stirred for a period of one to five days.

Operating Protocol B: Solid Nucleophile and Solid Arylation Agent

14.4 mg of cuprous oxide (0.1 mmoles), 116.8 mg of Chxn-Py-Al or another ligand as generally defined in this patent (0.4 mmoles), 3 mmoles of a nucleophilic compound, 2 mmoles of arylation agent and 1.303 g of caesium carbonate (4 mmoles) are successively introduced into a 35 ml Schlenk tube that has been oven dried at 100° C. and is provided with a magnetic stirrer (12×4.5 mm) and under a nitrogen atmosphere.

The Schlenk tube is purged under vacuum then refilled with nitrogen.

1.2 ml of acetonitrile or DMF is then added using a syringe.

The reactor is placed in an oil bath at a temperature of 82° C. and stirred for a period of one to five days.

Operating Protocol C: Liquid Nucleophile and Arylation Agent

14.4 mg of cuprous oxide (0.1 mmoles), 116.8 mg of Chxn-Py-Al or another ligand as generally defined in this patent (0.4 mmoles) and 1.303 g of caesium carbonate (4 mmoles) are successively introduced into a 35 ml Schlenk tube that has been oven dried at 100° C. and is provided with a magnetic stirrer (12×4.5 mm) and under a nitrogen atmosphere.

The Schlenk tube is purged under vacuum then refilled with nitrogen.

3 mmoles of a nucleophilic compound, 2 mmoles of arylation agent and 1.2 ml of acetonitrile or DMF are then added using syringes.

The reactor is placed in an oil bath at a temperature of 82° C. and stirred for a period of one to five days.

Operating Protocol D: Liquid Nucleophile and Solid Arylation Agent

14.4 mg of cuprous oxide (0.1 mmoles), 116.8 mg of Chxn-Py-Al or another ligand as generally defined in this patent (0.4 mmoles), 2 mmoles of a arylation agent and 1.303 g of caesium carbonate (4 mmoles) are successively introduced into a 35 ml Schlenk tube that has been oven dried at 100° C. and is provided with a magnetic stirrer (12×4.5 mm) and under a nitrogen atmosphere.

The Schlenk tube is purged under vacuum then refilled with nitrogen.

3 mmoles of nucleophilic compound and 1.2 ml of acetonitrile or DMF are then added using syringes.

The reactor is placed in an oil bath at a temperature of 82° C. and stirred for a period of one to five days.

Whichever operating protocol, A, B, C or D is used, the rest of the treatment is rigorously identical.

Determination of Isolated Yield:

After the period, the reaction mixture is diluted with 25 ml of dichloromethane, filtered through celite, concentrated completely under reduced pressure (about 20 mm of mercury) then taken up in 50 ml of dichloromethane.

This organic phase is extracted with distilled water (2×20 ml).

The aqueous phase is extracted again with 20 ml of dichloromethane.

The total organic phase is washed with a saturated aqueous sodium chloride solution (2×20 ml), dried over MgSO₄, filtered and concentrated under reduced pressure.

The residue obtained is purified by silica gel chromatography (35-70 μm).

Determination of Degree of Transformation:

After the period, 65 μl of 1,3-dimethoxybenzene (internal reference) is introduced into the cooled reaction medium, which is then diluted with 5 ml of diethyl ether or dichloromethane, depending on the solubility of the products to be analysed.

An aliquot is then removed, filtered through celite (or filter medium composed of about 90% SiO₂), eluting with diethyl ether or dichloromethane, extracted three times with distilled water then analysed by gas chromatography.

### Example 1.1

### Preparation of 1-phenyl-1H-pyrazole

Operating protocol A (82° C., 24 hours) was followed using 120.8 mg of Chxn-Thio-Al (0.4 mmoles), 211 μl of bromobenzene (2 mmoles), 204 mg of pyrazole (3 mmoles) and 1.2 ml of acetonitrile.

The residue obtained was purified by silica gel chromatography (eluent: dichloromethane/petroleum ether 60/40).

A colourless liquid was obtained in a yield of 80% by weight.

The compound obtained had the following formula:

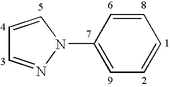

The characteristics were as follows:

B.Pt: 58° C., 0.2 mm Hg (Lit: 58-60° C., 0.2 mm Hg);

¹H NMR/CDCl₃ (250 MHz): δ 7.92 (dd, 1H, ³J_{HH}=2.4 Hz, ⁴J_{HH}=0.5 Hz, H₅), 7.70 (m, 3H, H_{3,6,9}), 7.45 (m, 2H, H_{2,8}), 7.29 (s, 1H, H₁), 6.46 (dd, 1H¹³J_{HH}=2.4 Hz, ³J_{HH}=1.8 Hz, H₄);

¹³C NMR/CDCl₃: δ 141.08 (C3), 140.23 (C7), 129.43 (C2 and C8), 126.75 (C5), 126.44 (C1), 119.21 (C6 and C9), 107.61 (C4);

GC/MS: Rt=15.37 min, M/Z=144, purity=100%;

Rf=0.40 (eluent: dichloromethane/petroleum ether, 60/40).

### Example 1.2

### Preparation of 1-phenyl-1H-pyrazole

Operating protocol A (82° C., 24 hours) was followed using 117 mg of Chxn-Py-Al (0.4 mmoles), 211 μl of bromobenzene (2 mmoles), 204 mg of pyrazole (3 mmoles) and 1.2 ml of acetonitrile.

The residue obtained was purified by silica gel chromatography (eluent: dichloromethane/petroleum ether 60/40).

A yield of 93.1% by weight of 1-phenyl-1H-pyrazole was obtained with formula:

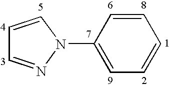

### Example 1.3

### Preparation of 1-phenyl-1H-pyrazole

Operating protocol A (82° C., 24 hours) was followed using 54 mg of Py-Alzone (0.4 mmoles), 211 μl of bromobenzene (2 mmoles), 204 mg of pyrazole (3 mmoles) and 1.2 ml of acetonitrile.

The residue obtained was purified by silica gel chromatography (eluent: dichloromethane/petroleum ether, 60/40).

A yield of 96.7% by weight of 1-phenyl-1H-pyrazole was obtained with formula:

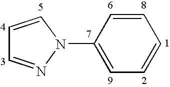

### Example 1.4

### Preparation of 1-phenyl-1H-pyrazole

Operating protocol A (82° C., 24 hours) was followed using 65.6 mg of N-Amido-Py-Alzone (0.4 mmoles), 211 μl of bromobenzene (2 mmoles), 204 mg of pyrazole (3 mmoles) and 1.2 ml of acetonitrile.

The residue obtained was purified by silica gel chromatography (eluent: dichloromethane/petroleum ether 60/40).

A yield of 95.2% by weight of 1-phenyl-1H-pyrazole was obtained with formula:

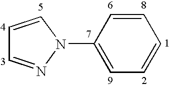

### Example 1.5

### Preparation of 1-phenyl-1H-pyrazole

Operating protocol A (82° C., 24 hours) was followed using 107.2 mg of Carbo-Py-Al (0.4 mmoles), 211 μl of bromobenzene (2 mmoles), 204 mg of pyrazole (3 mmoles) and 1.2 ml of acetonitrile.

The residue obtained was purified by silica gel chromatography (eluent: dichloromethane/petroleum ether 60/40).

A yield of 75% by weight of 1-phenyl-1H-pyrazole was obtained with formula:

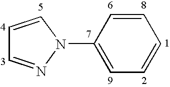

### Example 1.6

### Preparation of 1-(o-tolyl)-1H-pyrazole

Operating protocol A (82° C., 70 hours) was followed using 117 mg of Chxn-Py-Al (0.4 mmoles), 383 μl of 2-iodotoluene (3 mmoles), 136 mg of pyrazole (2 mmoles) and 1.2 ml of acetonitrile.

The degree of transformation and selectivity for 1-(o-tolyl)-1H-pyrazole were 100%.

The residue obtained was purified by silica gel chromatography (eluent: hexane/dichloromethane, 100/0 to 0/100).

297 mg of a pale yellow oil was obtained, corresponding to a yield of 94% by weight.

The compound obtained had the following formula:

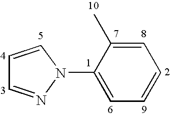

### Example 1.7

### Preparation of 1-(4′-bromophenyl)-1H-pyrazole

Operating protocol B (82° C., 72 hours) was followed using 117 mg of Chxn-Py-Al (0.4 mmoles), 1.887 g of 1,4-dibromobenzene (8 mmoles), 136 mg of pyrazole (3 mmoles) and 1.6 ml of acetonitrile.

The degree of transformation and selectivity for 1-(4′-bromophenyl)-1H-pyrazole were 89%.

The residue obtained was purified by silica gel chromatography (eluent: hexane/dichloromethane, 100/0 to 50/50).

366 mg of a colourless solid was obtained, corresponding to a yield of 82% by weight.

The compound obtained had the following formula:

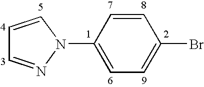

The characteristics were as follows:

M Pt: 71° C. (MeOH) (Lit: 70° C., aqueous MeOH obtained by Khan, M A; Lynch, B M; Hung, Y-Y; Can. J. Chem. 1963, 41, 1540-1547);

¹H NMR/CDCl₃ (250 MHz): δ 7.88 (dd, 1H, ³J_{HH}=2.5 Hz, ⁴J_{HH}=0.5 Hz, H₅), 7.72 (dd, 1H, ³J_{HH}=1.7 Hz, ⁴J_{HH}=0.5 Hz, H₃), 7.52-7.62 (m, 4H, H_{6,7,8,9}), 6.46 (dd, 1H, ³J_{HH}=1.7 Hz, ³J_{HH}=2.5 Hz, H₄);

¹³C NMR/CDCl₃: δ 141.41 (C3), 139.21 (C1), 132.46 (C6 and C7), 126.64 (C5), 120.59 (C8 and C9), 119.62 (C2), 108.83 (C4);

GC/MS: Rt=16.90 min, M/Z=222 and 224, purity=100%;

Rf=0.21 (eluent: hexane/dichloromethane, 50/50).

### Example 1.8

### Preparation of 1-(4-imidazol-1-yl-phenyl)-1H-pyrazole

Operating protocol B (82° C., 48 hours) was followed using 117 mg of Chxn-Py-Al (0.4 mmoles), 535 mg of 1-(4′-bromophenyl)-1H-imidazole (2.4 mmoles), 136 mg of pyrazole (2 mmoles) and 1.2 ml of acetonitrile.

The residue obtained was purified by silica gel chromatography (eluent: dichloromethane/methanol, 100/0 to 98/2).

387 mg of a colourless solid was obtained, corresponding to a yield of 92% by weight.

The compound obtained had the following formula:

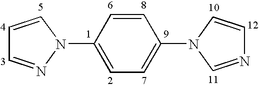

The characteristics were as follows:

M Pt: 174-176° C.;

¹H NMR/acetone-d₆ (250 MHz): δ 8.40 (dd, 1H, ³J_{HH}=2.5 Hz, ⁴J_{HH}=0.6 Hz, H₅), 8.15 (wide s, 1H, H₁₁), 7.98-8.04 (m, 2H, H_{7,8}), 7.73-7.79 (m, 2H, H_{2,6}), 7.73 (dd, 1H, ³J_{HH}=1.7 Hz, ⁴J_{HH}=0.6 Hz, H₃), 7.66 (wide s, 1H, H₁₀), 7.16 (wide s, 1H, H₁₂), 6.54 (dd, 1H, ³J_{HH}=1.7 Hz, ³J_{HH}=2.5 Hz, H₄);

¹³C NMR/DMSO-d₆: δ 141.28 (C3), 138.57 (C1), 135.27 (C11), 134.12 (C9), 127.92 (C5), 127.54 (C12), 121.75 (C2 and C6), 119.36 (C7 and C8), 118.68 (C10), 108.12 (C4);

GC/MS: Rt=22.49 min, M/Z=210, purity=100%;

Rf=0.22 (eluent: diethyl ether/methanol, 90/10).

### Example 1.9

### Preparation of 1H, 1′H-1,1′-p-phenylene-bis-pyrazole

Example 1.8 was repeated, replacing the 1-(4′-bromophenyl)-1H-imidazole with 1-(4′-bromophenyl)-1H-pyrazole (2.4 mmoles, 535 mg).

Pale yellow crystals were obtained which could be rendered colourless by re-crystallisation from chloroform.

The degree of transformation and isolated yield were 100%.

The compound obtained had the following formula:

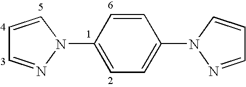

The characteristics were as follows:

M Pt: 180° C. (CHCl₃): (Lit: 182° C., CHCl₃ obtained by Kauffmann, T; Lexy, H., Chem. Ber. 1980, 113, 2749-2754);

¹H NMR/CDCl₃ (250 MHz): δ 7.95 (dd, 1H, ³J_{HH}=2.5 Hz, ⁴J_{HH}=0.6 Hz, H₅), 7.79 (s, 2H, H_{2,6}), 7.74 (dd, 1H, ³J_{HH}=1.6 Hz, ⁴J_{HH}=0.6 Hz, H₃), 6.49 (dd, 1H, ³J_{HH}=1.6 Hz, ³J_{HH}=2.5 Hz, H₄);

¹³C NMR/CDCl₃: δ 141.31 (C3), 138.41 (C1), 126.74 (C5), 120.04 (C2 and C6), 107.90 (C4);

GC/MS: Rt=21.28 min, M/Z=210, purity=98%;

Rf=0.38 (eluent: dichloromethane/ethyl acetate, 90/10).

### Example 1.10

### Preparation of 1-trans-styryl-1H-pyrazole

Operating protocol A (82° C., 24 hours) was followed using 117 mg of Chxn-Py-Al (0.4 mmoles), 387 μl of β-bromostyrene (3 mmoles trans/cis=91/9), 136 mg of pyrazole (2 mmoles) and 1.2 ml of acetonitrile.

The residue obtained was purified by silica gel chromatography (eluent: hexane/dichloromethane 100/0 to 50/50).

327 mg of a pale yellow solid was obtained, corresponding to a yield of 96% by weight.

The compound obtained had the following formula:

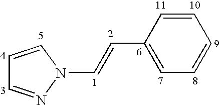

The characteristics were as follows:

M Pt: 53° C.;

H NMR/CDCl₃ (250 MHz): δ 7.66-7.67 (m, 2H, H_{3,5}), 7.52 (d, 1H, ³J_{HH}=14.5 Hz, H₁), 7.22-7.48 (m, 5H, H₇₋₁₁), 7.06 (d, 1H, ³J_{HH}=14.5 Hz, H₂), 6.40 (m, 1H, H₄). The value of the coupling constant ³J_{H1H2} proves that the phenyl and pyrazolyl substituents of the ethylenic bond are located in the trans position. The coupling constants ⁴J_{H3H5}, ³J_{H3H4} and ³J_{H4H5} could not be calculated as the signals were perturbed by coupling with the H₁ proton.

¹³C NMR/CDCl₃: δ 141.13 (C3), 135.09 (C6), 128.89 (C8 and C10), 128.14 (C1), 127.60 (C9), 126.48 (C5), 126.26 (C7 and C11), 116.88 (C2), 107.34 (C4);

GC/MS: Rt=17.05 min, M/Z=170, purity=98%;

Rf=0.22 (eluent: hexane/dichloromethane, 50/50).

### Example 1.11

### Preparation of 3,5-dimethyl-1-phenyl-1H-pyrazole

Operating protocol A (110° C., 54 hours) was followed using 117 mg of Chxn-Py-Al (0.4 mmoles), 336 μl of iodobenzene (3 mmoles), 192 mg of 3,5-dimethylpyrazole (2 mmoles) and 1.2 ml of DMF. The degree of transformation and selectivity for 5-dimethyl-1-phenyl-1H-pyrazole were 100%.

The residue obtained following the treatment was purified by silica gel chromatography (eluent: hexane/dichloromethane, 100/0 to 10/90).

323 mg of a yellow oil was obtained, corresponding to a yield of 94% by weight.

The compound obtained had the following formula:

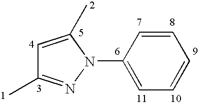

The characteristics were as follows:

¹H NMR/CDCl₃ (250 MHz): δ 7.25-7.40 (m, 5H, H_{7,8,9,10,11}), 5.95 (broad s, 1H, H₄), 2.27 (d, ⁴J_{HH}=0.8 Hz, 3H, H₂), 2.25 (broad s, 3H, H₁). Only the coupling constant between the protons of the methyl group located in the 5 position and H₄ could be determined. The coupling constant between the protons of the methyl group located in the 3 position and H₄ was too small to be read;

¹³C NMR/CDCl₃: δ 148.86 (C3), 140.00 (C6), 139.28 (C5), 128.93 (C8 and C10), 127.14 (C9), 124.69 (C7 and C11), 106.92 (C4), 13.48 (C2), 12.31 (C1) [De la Hoz, A; Pardo, M C; Elguero, J; Fruchier, A; Magn. Reson. Chem. 1989, 27, 603-606].

GC/MS: Rt=15.30 min, M/Z=172, purity=99%;

Rf=0.17 (eluent: dichloromethane).

### Example 1.12

### Preparation of 3,5-dimethyl-1-phenyl-1H-pyrazole

Operating protocol A (110° C., 24 hours) was followed using 117 mg of Chxn-Py-Al (0.4 mmoles), 336 μl of iodobenzene (3 mmoles), 192 mg of 3,5-dimethylpyrazole (2 mmoles) and 1.2 ml of DMF.

The degree of transformation and selectivity for 5-dimethyl-1-phenyl-1H-pyrazole were 75%.

The compound obtained had the following formula:

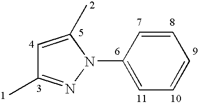

### Example 1.13

### Preparation of 1-phenyl-1H-pyrazole

Operating protocol A (82° C., 48 hours) was followed using 117 mg of Chxn-Py-Al (0.4 mmoles), 211 μl of bromobenzene (2 mmoles), 204 mg of imidazole (3 mmoles) and 1.2 ml of acetonitrile.

The yellow oil obtained following treatment was purified by silica gel chromatography (eluent: dichloromethane/ethyl acetate, 100/0 to 50/50).

A pale yellow oil corresponding to 1-phenyl-1H-imidazole was obtained in a yield of 80% and had formula:

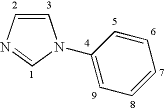

The characteristics were as follows:

¹H NMR/CDCl₃: δ (Collman, J P; Zhong, M; Org. Lett. 2000, 2, 1233-1236, Supporting Information) 7.84 (dd, 1H, ⁴J_{HH}=1.3 Hz, ⁴J_{HH}=1.0 Hz, H₁), 7.43-7.53 (m, 2H, H_{6,8}), 7.32-7.41 (m, 3H, H_{5,7,9}), 7.28 (t, 1H, ³J_{HH}=1.3 Hz, ⁴J_{HH}=1.3 Hz, H₃), 7.19 (dd, 1H, ³J_{HH}=1.3 Hz, ⁴J_{HH}=1.0 Hz, H₂);

¹³C NMR/acetone-d₆: δ 138.46 (C4), 136.37 (C1), 131.16 (C2), 130.77 (C6 and C8), 127.88 (C7), 121.69 (C5 and C9), 118.77 (C3);

GC/MS: Rt=14.76 min, M/Z=144, purity=100%;

Rf=0.17 (eluent: dichloromethane/ethyl acetate, 50/50).

### Example 1.14

### Preparation of 1-(4′-trifluoromethylphenyl-1H-imidazole

General procedure A (82° C., 48 hours) was followed using 72 mg of cuprous oxide (0.5 mmoles), 585 mg of Chxn-Py-Al (2 mmoles), 1.40 ml of 4-bromotrifluormethylbenzene (10 mmoles), 1.02 g of imidazole (15 mmoles), 5.86 g of caesium carbonate (18 mmoles) and 6 ml of acetonitrile.

The residue obtained was purified by silica gel chromatography (eluent: hexane/dichloromethane, 100/0 to 0/100).

359 mg of a pale yellow solid was obtained in a yield of 85%.

The compound obtained had the following formula:

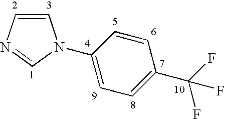

The characteristics were as follows:

MPt: 70° C.;

¹H NMR/CDCl₃: δ 7.90 (wide s, 1H, H₁), 7.72 (m, 2H, H_{5,9}), 7.49 (m, 2H, H_{6,8}), 7.31 (wide s, 1H, H₃), 7.22 (s, 1H, H₂);

¹³C NMR/DMSO-d₆: δ 8.43 (wide s, 1H, H₁), 7.85-7.95 (m, 5H, H_{3,5,6,8,9}), 7.22 (s, 1H, H₂);

¹³C NMR/CDCl₃: 139.99 (C4), 135.52 (C1), 131.20 (C2), 129.47 (q, ²J_{CF}=33.2 Hz, C7), 127.23 (q, ³J_{CF}=3.8 Hz, C6 and C8), 123.63 (q, ¹J_{CF}=272.1 Hz, C10), 121.25 (C5 and C9), 118.26 (C3);

¹⁹F NMR/CDCl₃: δ −62.92 (CF₃);

GC/MS: Rt=14.82 min, M/Z=212, purity=98%;

Rf=0.20 (eluent: dichloromethane).

### Example 1.15

### Preparation of 1-phenyl-1H-indole

Operating protocol A (82° C., 24 hours) was followed using 117 mg of Chxn-Py-Al (0.4 mmoles), 224 μl of iodobenzene (2 mmoles), 351 mg of indole (3 mmoles) and 1.2 ml of acetonitrile.

The degree of transformation and selectivity for 1-phenyl-1H-indole were 99.5%.

The red oil obtained following treatment was purified by silica gel chromatography (eluent: hexane/dichloromethane, 100/0 to 50/50).

A yellow-green oil was obtained in a yield of 92%.

The compound obtained had the following formula:

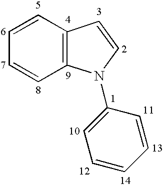

The characteristics were as follows:

¹H NMR/CDCl₃: δ 7.74-7.80 (m, 1H, H₅), 7.62-7.68 (m, 1H, H₈), 7.51-7.58 (m, 4H, H_{10,11,12,13}), 7.34-7.47 (m, 1H, H₁₄), 7.40 (d, ³J_{HH}=3.3 Hz, 1H, H₂); 7.20-7.33 (m, 2H, H_{6,7}), 6.76 (dd, 1H, ³J_{H3H2}=3.3 Hz, ³J_{H3H8}=0.9 Hz, H₃). The attributions were made by means of a COSY H—H experiment.

¹³C NMR/CDCl₃: 139.90 (C1), 135.93 (C9), 129.67 (C10 and C11), 129.41 (C4), 128.02 (C14), 126.50 (C2), 124.44 (C12 and C13), 122.43 (C6), 121.21 (C5), 120.43 (C7), 110.58 (C8), 103.65 (C3).

GC/MS: M/Z=193, purity=100%;

Rf=0.23 (eluent: hexane).

### Example 1.16

### Preparation of 1-phenyl-1H-indole

Operating protocol A (50° C., 74 hours) was followed using 117 mg of Chxn-Py-Al (0.4 mmoles), 224 μl of iodobenzene (2 mmoles), 351 mg of indole (3 mmoles) and 1.2 ml of acetonitrile.

The degree of transformation and selectivity for 1-phenyl-1H-indole was 99%.

The compound obtained had the following formula:

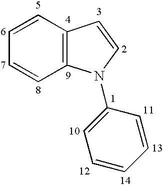

### Example 1.17

### Preparation of 1-phenyl-1H-[1,2,4]triazole

Operating protocol A (82° C., 48 hours) was followed using 117 mg of Chxn-Py-Al (0.4 mmoles), 336 μl of iodobenzene (3 mmoles), 138 mg of 1,2,4-triazole (2 mmoles), 1.042 g of caesium carbonate (3.2 mmoles) and 1.2 ml of DMF.

The degree of transformation and selectivity were 100% and 98% respectively.

The residue obtained following treatment was purified by silica gel chromatography (eluent: hexane/dichloromethane, 100/0 to 50/50).

264 mg of a dark yellow solid was obtained in a yield of 91%.

Pale yellow needles were obtained after re-crystallisation from chloroform.

The compound obtained had the following formula:

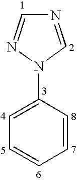

The characteristics were as follows:

MPt: 46° C. (CHCl₃) (Lit: 46-47° C. given by Micetich, R G; Spevak, P; Hall, T W; Bains, B K; Heterocycles 1985, 23, 1645-1649);

¹H NMR/CDCl₃: δ8.52 (wide s, 1H, H₁), 8.04 (wide s, 1H, H₂), 7.53-7.65 (m, 2H, H_{4,8}), 7.26-7.51 (m, 3H, H_{5,6,7});

¹³C NMR/CDCl₃: δ 152.55 (C1), 140.88 (C2), 139.96 (C3), 129.73 (C5 and C7), 128.15 (C6), 119.99 (C4 and C8);

GC/MS: Rt=14.02 min, M/Z=145, purity=100%;

Rf=0.21 (eluent: dichloromethane/ethyl acetate, 90/10).

### Example 1.18

### Preparation of 1-phenyl-1H-[1,2,4]triazole

Operating protocol A (82° C., 24 hours) was followed using 117 mg of Chxn-Py-Al (0.4 mmoles), 336 μl of iodobenzene (3 mmoles), 138 mg of 1,2,4-triazole (2 mmoles), 1.042 g of caesium carbonate (3.2 mmoles) and 1.2 ml of DMF.

The degree of transformation and selectivity were 79% and 99% respectively.

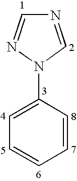

### Example 1.19

### Preparation of 1-phenyl-1H-[1,2,4]triazole

Example 1.18 was repeated, operating at 50° C. (72 hours). The degree of transformation and selectivity for 1-phenyl-1H-[1,2,4-triazole] were 75% and 99% respectively.

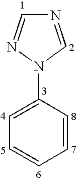

### Example 1.20

### Preparation of 1-phenyl-1H-pyrrole

Operating protocol C (50° C., 4 days) was followed using 117 mg of Chxn-Py-Al (0.4 mmoles), 269 μl of iodobenzene (2.4 mmoles), 208 μl of pyrrole (2 mmoles) and 1.2 ml of acetonitrile.

The residue obtained was purified by silica gel chromatography (eluent: hexane).

The yield and degree of transformation of 1-phenyl-1H-pyrrole were 100%.

The compound obtained had the following formula:

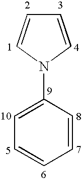

The characteristics were as follows:

MPt: 62° C. (Lit: 62° C. obtained by Dumoulin, H; Rauly, S; Robba, M; J. Heterocycl. Chem. 1995, 32, 1703-1707);

¹H NMR/CDCl₃: δ 7.50-7.60 (m, 4H, H_{5,7,8,10}), 7.38 (m, 1H, H₆), 7.26 (m, 2H, H_{1,4}), 6.54 (m, 2H, H_{2,3});

¹³C NMR/CDCl₃: δ 140.96 (C9), 129.71 (C5 and C7), 125.74 (C6), 120.64 (C8 and C10), 119.44 (C1 and C4), 110.68 (C2 and C3);

GC/MS: Rt=12.75 min, M/Z=143, purity=99%;

Rf=0.33 (eluent: hexane).

### Example 1.21

### Preparation of 1-phenyl-1H-pyrrole

Operating protocol C (82° C., 4 days) was followed using 117 mg of Chxn-Py-Al (0.4 mmoles), 253 μl of bromobenzene (2.4 mmoles), 208 μl of pyrrole (2 mmoles) and 1.2 ml of acetonitrile.

The residue obtained was purified by silica gel chromatography (eluent: hexane).

The degree of transformation of 1-phenyl-1H-pyrrole was 70%.

The compound obtained had the following formula:

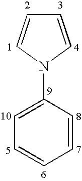

### Example 1.22

### Preparation of 1-(4′-aminophenyl)-1H-pyrazole

General procedure B (82° C., 42 hours) was followed using 117 mg of Chxn-Py-Al (0.4 mmoles), 516 mg of 4-bromoaniline (3 mmoles), 136 mg of pyrazole (2 mmoles) and 1.2 ml of acetonitrile.

The brown oil obtained after the filtration step was purified directly by alumina chromatography (eluent: hexane/dichloromethane, 100/0 to 50/50).

290 mg of an orange solid was obtained, corresponding to a yield of 91%.

The treatment and analyses were carried out as quickly as possible protected from the light as there was a risk that the compound would decompose.

The compound obtained had the following formula:

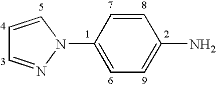

The characteristics were as follows:

MPt: 42-43° C.;

¹H NMR/CDCl₃ (250 MHz): δ 7.75 (dd, 1H, ³J_{HH}=2.4 Hz, ⁴J_{HH}=0.5 Hz, H₅), 7.66 (dd, 1H, ³J_{HH}=1.8 Hz, ??=0.5 Hz, H₃), 7.40 (m, 2H, H_{6,7}), 6.66 (m, 2H, H_{8,9}), 6.38 (dd, 1H, ³J_{HH}=?? Hz, ³J_{HH}=2.4 Hz, H₄) 3.79 (s, 2H, NH₂). Purity=98%;

¹³C NMR/CDCl₃: δ 145.47 (C2), 140.22 (C3), 132.31 (C1), 126.80 (C5), 121.10 (C6, C7), 115.43 (C8, C9), 106.83 (C4);

GC/MS: Rt=17.77 min, M/Z=159;

Rf=0.17 (eluent: dichloromethane/ethyl acetate, 95/5, silica) or 0.17 (eluent: dichloromethane/hexane, 50/50, alumina).

### Example 1.23

### Preparation of 1-methyl-4-(1H-pyrazol-1′-yl)-1H-pyrazole

The preceding example was repeated, using pyrazole and 1-methyl-4-bromopyrazole.

The compound obtained had the following formula:

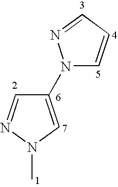

The characteristics were as follows:

M.Pt: 63-64° C.;

¹H NMR/acetone-d₆ (250 MHz): δ 8.00 (dd, 1H, ³J_{HH}=2.4 Hz, ⁴J_{HH}=0.65 Hz, H₅), 8.00 (d, 1H, ⁴J_{HH}=0.75 Hz, H₇), 7.77 (d, 1H, ⁴J_{HH}=0.75 Hz, H₂), 7.60 (dd, 1H, ³J_{HH}=1.85 Hz, ⁴J_{HH}=0.65 Hz, H₃), 6.41 (dd, 1H, ³J_{HH}=1.85 Hz, ³J_{HH}=2.4 Hz, H₄), 3.94 (s, 3H, H₁);

¹³C NMR/CDCl₃: δ 140.34 (C3), 130.55 (C5), 127.98 (C2), 126.30 (C6), 121.93 (C7), 106.68 (C4), 39.51 (C1);

GC/MS: Rt=14.13 min, M/Z=148, purity=99%;

FAB+ (NBA matrix): 149 (100%, M+H⁺), 55 (24%), 148 (22%), 69 (20%, pyrazole+H⁺), 297 (3%, 2M+1);

HRMS: Calculated for C₇H₉N₄ (M⁺+H): 149.0827. Found: 149.0819;

Rf: 0.28 (eluent: dichloromethane/methanol, 98/2).

### Example 1.24

### Preparation of 1-phenyl-3-trifluoromethyl-5-(p-tolyl)-1H-pyrazole

This compound was isolated by silica gel chromatography following arylation of 3-trifluoromethyl-5-(p-tolyl)-1H pyrazole using iodobenzene as described in Example 1.1.

The compound obtained had the following formula:

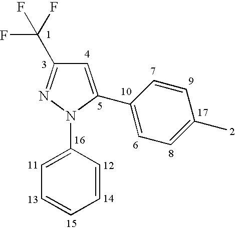

The characteristics were as follows:

¹H NMR/acetone-d₆: δ 7.39-7.46 (m, 3H, H_{13,14,15}), 7.33-7.38 (m, 2H, H_{11,12}), 7.19 (m, 4H, H₆₋₉), 6.94 (q, 1H, ⁴J_{HF}=0.6 Hz, H₄), 2.32 (s, 3H, H₂);

¹³C NMR/acetone-d₆: δ 146.01 (C5), 143.32 (q, ²J_{CF}=38.0 HZ, C3), 140.48 (C17), 139.95 (C16), 130.16 (C13 and C14), 129.99 (C8 and C9), 129.65 (C6 and C7), 129.44 (C15), 127.20 (C10), 126.51 (C11 and C12), 122.64 (q, ¹J_{CF}=268.3 Hz, C1), 106.01 (q, ³J_{CF}=1.9 Hz, C4), 21.20 (C2). Carbons 6-9 have similar chemical displacements, which agrees with the fact that the signals for protons 6-9 are superimposed;

¹⁹F NMR/acetone-d₆: δ −63.05 (d, ⁴J_{HF}=0.6 Hz), purity=99.8%;

GC/MS: Rt=20.54 min, M/Z=302, purity=99.5%;

Rf: 0.30 (eluent: hexane/dichloromethane, 80/20).

### Example 1.25

### Preparation of 1-phenyl-3-(p-tolyl)-5-trifluoromethyl-1H-pyrazole

As described for the preceding example, this compound was isolated by arylation of 3-trifluoromethyl-5-(p-tolyl)-1H pyrazole using iodobenzene.

The compound obtained had the following formula:

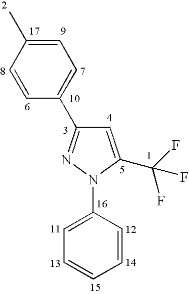

The characteristics were as follows:

¹³C NMR/acetone-d₆: δ 152.44 (C3), 140.12 (q, ²J_{CF}=18.2 Hz, C5), 139.31 (C16), 134.48 (C17), 130.26 (C8, C9 and C15), 130.06 (C13 and C14), 127.21 (C10), 126.64 (q, ⁶J_{CF}=0.4 Hz, C6 and C7) 126.48 (C11 and C12), 120.95 (q, ¹J_{CF}=268.3 Hz, C1), 106.01 (q, ³J_{CF}=2.6 Hz, C4), 21.23 (C2);

¹⁹F NMR/acetone-d₆: 6-58.51 (s);

GC/MS: Rt=21.16 min, M/Z=302, purity=98%;

Rf: 0.34 (eluent: hexane/dichloromethane, 80/20).

### Example 1.26

### Preparation of 5-(3-chlorosulphonyl-4-methylphenyl)-1-phenyl-3-trifluoromethyl-1H-pyrazole

As described for in Example 1.24, this compound was obtained by arylation of 5-(3-chlorosulphonyl-4-methylphenyl)-3-trifluoromethyl-1H pyrazole using iodobenzene.

The compound obtained had the following formula:

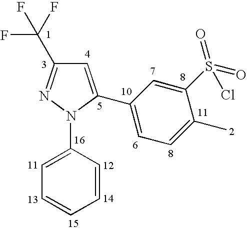

The characteristics were as follows:

¹H NMR/CDCl₃: δ 7.94 (m, 1H, H₇), 7.40-7.47 (m, 3H), 7.37-7.39 (m, 2H), 7.27-7.35 (m, 2H), 6.87 (m, 1H, H₄), 2.78 (s, 3H, H₂). Purity: 95%;

GC/MS: Rt=25.92 min, M/Z=400 and 402;

Rf: 0.24 (eluent: hexane/dichloromethane, 80/20).

### Example 1.27

### Preparation of 1-phenyl-1H-pyrazole

The complex CuI/Chxn-Py-Al (0.2 mmole) synthesised according to the protocol given before Example 1, 1.303 mg of caesium carbonate (4 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen.

204 mg of pyrazole (3 mmole), 224 μl of iodobenzene (2 mmole), 1.2 ml of acetonitrile and 600 mg of 3 Å activated molecular sieve were then added.

The reactor was placed in an oil bath at a temperature of 50° C. and stirred for 24 hours.

The residue obtained was directly purified by silica column chromatography (eluent: dichloromethane/hexane 70/30).

1-phenyl-1H-pyrazole was obtained in a yield of 90%.

### Example 2

### N-Arylation of Amides, Carbamates and Derivatives General Operating Protocol

The following are successively introduced into a 35 ml Schlenk tube placed in a nitrogen atmosphere:
cuprous oxide (0.1 mmoles);
ligand (0.4 mmoles);
nucleophilic compound (3 mmoles);
a base (4 mmoles);
2 mmoles of arylation agent;
and 1.2 ml of acetonitrile or DMF

The mixture is placed in an oil bath at a temperature of 82° C. and stirred for 24 hours.

After this period, the mixture is diluted with ethyl ether or dichloromethane.

Determination of Isolated Yield:

After the period, the reaction mixture is diluted with 25 ml of dichloromethane, filtered through celite, concentrated completely under reduced pressure then taken up in 50 ml of dichloromethane.

This organic phase is extracted with distilled water (2×20 ml).

The aqueous phase is extracted again with 20 ml of dichloromethane.

The total organic phase is washed with a saturated aqueous sodium chloride solution (2×20 ml), dried over MgSO₄, filtered and concentrated under reduced pressure.

The residue obtained is purified by silica gel chromatography (35-70 μm).

Determination of Degree of Transformation:

After the period, 65 μl of 1,3-dimethoxybenzene (internal reference) is introduced into the cooled reaction medium, which is then diluted with 5 ml of diethyl ether or dichloromethane, depending on the solubility of the products to be analysed.

An aliquot is then removed, filtered through celite, eluting with diethyl ether or dichloromethane, extracted three times with distilled water then analysed by gas chromatography.

### Example 2.1

### Preparation of 3-phenyloxazolidin-2-one

14.4 mg of cuprous oxide (0.1 mmoles), 117 mg of Chxn-Py-Al (0.4 mmoles), 263 mg of oxazolidin-2-one (3 mmoles), 1.043 g of caesium carbonate (3.2 mmoles) and 600 mg of ground and activated 3 Å molecular sieve (KnNa_{12-n[(AlO}₂)₁₂(SiO₂)₁₂]) were successively introduced into a 35 ml Schlenk tube that had been oven dried at 100° C. and provided with a magnetic stirrer (12×4.5 mm) and under a nitrogen atmosphere.

The Schlenk tube was purged under vacuum then refilled with nitrogen.

224 μl of iodobenzene (2 mmoles) then 1.2 ml of DMF were then added using syringes.

The reactor was placed in an oil bath at a temperature of 82° C. and stirred for a period of 24 hours.

The degree of transformation of 3-phenyloxazolidin-2-one was 99.7% and the selectivity reached 100%.

After the period, the reaction mixture was diluted with 25 ml of dichloromethane, filtered through celite, concentrated completely under reduced pressure then taken up in 50 ml of dichloromethane.

This organic phase was extracted with distilled water (2×20 ml).

The aqueous phase is extracted again with 20 ml of dichloromethane.

The total organic phase was washed with a saturated aqueous sodium chloride solution (2×20 ml), dried over MgSO₄, filtered and concentrated under reduced pressure.

The residue obtained was purified by silica gel chromatography (eluent: hexane/dichloromethane, 50/50 to 0/100).

316 mg of a colourless solid was obtained, corresponding to a yield of 97%.

The compound obtained had the following formula:

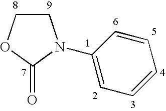

The characteristics were as follows:

M.Pt: 120° C. (Lit: 120-121° C., given by Gulbins, E; Hamann, K; Chem. Ber. 1966, 99, 55-61);

¹H NMR/CDCl₃: δ 7.48-7.53 (m, 2H, H_{3,5}), 7.30-7.38 (m, 2H, H_{2,6}), 7.07-7.15 (m, 1H, H₄), 4.40 (m, 2H, H₈, ³J_{HH}=8.00 Hz), 3.97 (m, 2H, Hg, ³J_{HH}=8.0 Hz);

¹³C NMR/CDCl₃: δ 155.34 (C7), 138.30 (C1), 129.04 (C3 and C5), 124.01 (C4), 118.22 (C2 and C6), 61.37 (C8), 45.14 (C9);

GC/MS: Rt=18.25 min, M/Z=163, purity=100%;

Rf: 0.29 (eluent: dichloromethane).

### Example 2.2

### Preparation of 3-phenyloxazolidin-2-one

Example 2.1 was repeated, heating for 96 h at 50° C.

The degree of transformation of 3-phenyloxazolidin-2-one was 99.6% and the selectivity reached 100%.

### Example 2.3

### Preparation of 1-phenyl-1H-pyridin-2-one

Example 2.1 was repeated, using 72 mg of cuprous oxide (0.5 mmoles), 584 mg of Chxn-Py-Al (2 mmoles), 951 mg of 2-hydroxypyridine (10 mmoles), 6.52 g of caesium carbonate (20 mmoles), 3 g of ground and activated 3 Å molecular sieve, 1.68 ml of iodobenzene (15 mmoles) and 6 ml of acetonitrile.

The degree of transformation of 1-phenyl-1H-pyridin-2-one was 98%.

The residue obtained was purified by silica gel chromatography (eluent: hexane/dichloromethane/ethyl acetate, 100/0/0 to 0/100/0 then 0/100/0 to 0/80/20).

1.54 g of a yellow solid was obtained, corresponding to a yield of 90%.

The compound obtained had the following formula:

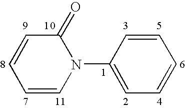

The characteristics were as follows:

M.Pt: 127° C. (Lit: 129° C., diisopropyl ether, given by Ukita, T; Sugahara, M; Chem. Pharm. Bull. 1997, 45, 719-721);

¹H NMR/DMSO-d₆: δ 7.59-7.66 (m, 1H, H₁), 7.36-7.56 (m, 6H, H_{2-6,8}), 6.48 (m, 1H, H₉), 6.31 (m, 1H, H₇);

¹³C NMR/CDCl₃: δ 162.41 (C10), 140.97 (C1), 139.88 (C11), 138.01 (C8), 129.34 (C4 and C5), 128.48 (C6), 126.54 (C2 and C3), 121.91 (C9), 105.93 (C7);

GC/MS: Rt=18.11 min, M/Z=171, purity=99%;

Rf: 0.14 (eluent: dichloromethane/ethyl acetate, 90/10).

### Example 2.4

### Preparation of benzanilide(N-phenylbenzamide)

Example 2.1 was repeated, replacing the oxazolidin-2-one with 363 mg of benzamide (3 mmoles) and taking the reaction time to 48 h.

The degree of transformation of N-phenylbenzamide was 96% and the selectivity reached 100%.

The residue obtained was purified by silica gel chromatography (eluent: hexane/dichloromethane, 50/50 to 100/0).

359 mg of a colourless solid was obtained, corresponding to a yield of 91%.

The compound obtained had the following formula:

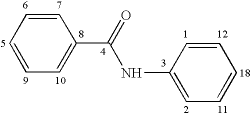

The characteristics were as follows:

M.Pt: 164° C. (Lit: 163° C., EtOH, given by Goswami, B N; Borthakur, N, Ghosh, A C; J. Chem. Research (S), 1998, 268-269);

¹H NMR/CDCl₃: δ 7.88 (wide s, 1H, NH), 7.86 (m, 2H, H_{7,10}), 7.64 (m, 2H, H_{6,9}), 7.32-7.58 (m, 5H, H_{1,2,5,11,12}) 7.15 (m, 1H, H₁₃). Purity=99%;

¹³C NMR/CDCl₃: δ 165.81 (C4), 137.96 (C3), 135.03 (C8), 131.83 (C5), 129.09 (C11 and C12), 128.78 (C7 and C10), 127.04 (C6 and C9), 124.58 (C13), 120.27 (C1 and C2);

GC/MS: Rt=20.76 min, M/Z=197;

Rf: 0.45 (eluent: dichloromethane).

### Example 2.5

### Preparation of 1-phenylpyrrolidin-2-one

Example 2.1 was repeated, replacing the oxazolidin-2-one with 152 μl of pyrrolidin-2-one (2 mmoles) and operating with 336 μl of iodobenzene (3 mmoles), the latter being added at the same time as the pyrrolidin-2-one.

The reaction time was taken to 40 h.

The degree of transformation and selectivity for 1-phenylpyrrolidin-2-one were 100%.

The residue obtained was purified by silica gel chromatography (eluent: hexane/dichloromethane/ethyl acetate, 50/50/0 to 0/95/5).

297 mg of a colourless solid was obtained, corresponding to a yield of 92%.

The compound could also be isolated by re-crystallising the residue obtained from the solvent extraction steps from ethanol rather than using silica chromatography.

265 mg of a beige solid was obtained, corresponding to a yield of 82%.

The compound obtained had the following formula:

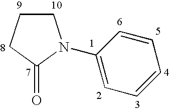

The characteristics were as follows:

M.Pt: 69-70° C. (Lit: 70° C., diisopropyl ether, given by Ukita, T; Sugahara, M; Chem. Pharm. Bull. 1997, 45, 719-721);

¹H NMR/CDCl₃: δ 7.58-7.63 (m, 2H, H_{2,6}), 7.32-7.40 (m, 2H, H_{3,5}), 7.13-7.18 (m, 1H, H₄), 3.87 (m, 2H, H₁₀) 2.61 (m, 2H, H₈), 2.08-2.23 (m, 2H, H₉);

¹³C NMR/CDCl₃: δ 174.20 (C7), 139.43 (C1), 128.81 (C2 and C6), 124.48 (C4), 119.96 (C3 and C5), 48.78 (C10), 32.76 (C8), 18.03 (C9);

GC/MS: Rt=17.38 min, M/Z=161, Purity=99%;

Rf: 0.53 (eluent: dichloromethane/ethyl acetate, 80/20).

### Example 2.6

### Preparation of N-phenylbenzenesulphonamide

Example 2.1 was repeated, using 14.4 mg of cuprous oxide (0.1 mmoles), 117 mg of Chxn-Py-Al (0.4 mmoles), 472 mg of benzenesulphonamide (3 mmoles), 224 μl of iodobenzene (2 mmoles), 1.04 g of caesium carbonate (3.2 mmoles), 600 mg of ground and activated 3 Å molecular sieve and 1.6 ml of DMF.

The reaction time was taken to 48 h.

The degree of transformation of N-phenylbenzenesulphonamide was 95%.

After this reaction period, the reaction mixture was diluted with 25 ml of dichloromethane/methanol and filtered through celite.

The residue obtained was purified by silica gel chromatography (eluent: hexane/dichloromethane, 90/10 to 5/95).

411 mg of a colourless solid was obtained, corresponding to a yield of 88%.

The compound obtained had the following formula:

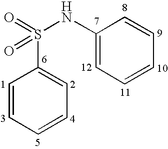

The characteristics were as follows: M.Pt: 109-110° C. (Lit: 110° C., given by Hellwinkel, D; Supp, M; Chem. Ber. 1976, 109, 3749-3766);

¹H NMR/CDCl₆: δ 7.78-7.88 (m, 2H, H_{1,2}), 7.79 (broad s, 1H, NH), 7.35-7.50 (m, 3H, H₃₋₅), 7.07-7.25 (m, 5H, H₈₋₁₂);

¹³C NMR/CDCl₃: δ 138.89 (C6), 136.58 (C7), 133.10 (C5), 129.34 (C3 and C4), 129.10 (C9 and C11), 127.29 (C1 and C2), 125.33 (C10), 121.55 (C8 and C12);

GC/MS: Rt=21.54 min, M/Z=233, purity=99%;

Rf: 0.36 (eluent: dichloromethane).

### Example 3

### Arylation of Phenols

### General Operating Protocol

The following are successively introduced into a 35 ml Schlenk tube placed in a nitrogen atmosphere:
Cuprous oxide (0.1 mmoles); ligand (0.4 mmoles);
nucleophilic compound (2 mmoles);
a base (4 mmoles);
3 mmoles of arylation agent;
and 1.2 ml of acetonitrile.

The mixture is placed in an oil bath at a temperature of 82° C. and stirred for 24 hours.

Determination of Isolated Yield:

After the period, the reaction mixture is diluted with 25 ml of dichloromethane, filtered through celite, concentrated completely under reduced pressure then taken up in 50 ml of dichloromethane.

This organic phase is extracted with distilled water (2×20 ml).

The aqueous phase is extracted again with 20 ml of dichloromethane.

The total organic phase is washed with a saturated aqueous sodium chloride solution (2×20 ml), dried over MgSO₄, filtered and concentrated under reduced pressure.

The residue obtained is purified by silica gel chromatography (35-70 μm).

Determination of Degree of Transformation:

After the period, 65 μl of 1,3-dimethoxybenzene (internal reference) is introduced into the cooled reaction medium, which is then diluted with 5 ml of diethyl ether or dichloromethane, depending on the solubility of the products to be analysed.

An aliquot is then removed, filtered through celite, eluting with diethyl ether or dichloromethane, extracted three times with distilled water then analysed by gas chromatography.

### Example 3.1

Preparation of Diphenyl Ether

14.4 mg of cuprous oxide (0.1 mmoles), 117 mg of Chxn-Py-Al (0.4 mmoles), 188 mg of phenol (2 mmoles), 1.303 g of caesium carbonate (4 mmoles) and 600 mg of ground and activated 3 Å molecular sieve (KₙNa₁₂₋ₙ[(AlO₂)₁₂(SiO₂)₁₂]) were successively introduced into a 35 ml Schlenk tube that had been oven dried at 100° C. and provided with a magnetic stirrer (12×4.5 mm) and under a nitrogen atmosphere.

The Schlenk tube was purged under vacuum then refilled with nitrogen.

336 μl of iodobenzene (3 mmoles) then 1.2 ml of acetonitrile were added using syringes.

The reactor was placed in an oil bath at a temperature of 82° C. and stirred for a period of 24 hours.

The degree of transformation and the selectivity for diphenyl ether were 100%.

After this period, the reaction mixture was diluted with 25 ml of dichloromethane, filtered through celite, concentrated completely under reduced pressure then taken up in 50 ml of dichloromethane.

This organic phase was extracted with distilled water (2×20 ml).

The aqueous phase was extracted again with 20 ml of dichloromethane.

The total organic phase was washed with a saturated aqueous sodium chloride solution (2×20 ml), dried over MgSO₄, filtered and concentrated under reduced pressure.

The oily residue obtained after treatment was complete was purified by silica gel chromatography (eluent: hexane).

344 mg of a colourless oil was obtained (corresponding to a yield of 100%), which crystallised out after a few hours in the refrigerator (colourless crystals).

The compound obtained had the following formula:

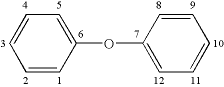

The characteristics were as follows:

M.Pt: 26° C. (Lit: 85° C., given by Byers, C H; Williams, D F; J. Chem. Eng. Data 1987, 32, 344-348);

¹H NMR/CDCl₃: δ 7.37-7.47 (m, 4H, H_{2,4,9,11}), 7.10-7.23 (m, 6H, H_{1,3,5,8,10,12});

¹³C NMR/CDCl₃: δ 157.38 (C6 and C7), 129.88 (C2, C4, C9 and C11), 123.35 (C3 and C10), 119.02 (C1, C5, C8 and C12);

GC/MS: Rt=14.43 min, M/Z=170, purity=99%;

Rf: 0.33 (eluent: hexane).

### Example 3.2

### Preparation of 4-methoxyphenyl phenyl ether

Example 3.1 was repeated, replacing the phenol with 248 mg of 4-methoxyphenol (2 mmoles) and heating for 28 h at 82° C.

The degree of transformation and the selectivity for 4-methoxyphenyl phenyl ether were 100%.

The orange oil obtained after treatment was complete was purified by silica gel chromatography (eluent: hexane/dichloromethane, 100/0 to 95/5).

380 mg of a colourless oil was obtained, which corresponded to a yield of 95%.

The compound obtained had the following formula:

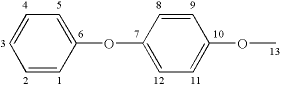

The characteristics were as follows:

¹H NMR/CDCl₃: δ 7.30-7.39 (m, 2H, H_{2,4}), 6.89-7.09 (m, 7H, H_{1,3,5,8,9,11,12}), 3.84 (s, 3H, H₁₃);

³C NMR/CDCl₃: δ 158.60 (C6), 155.97 (C10), 150.18 (C7), 129.69 (C2 and C4), 122.49 (C3), 120.91 (C8 and C12), 117.64 (C1 and C5), 114.92 (C9 and C11), 55.67 (C13);

GC/MS: Rt=17.67 min, M/Z=200, purity=95.5%;

Rf: 0.25 (eluent: hexane/dichloromethane, 80/20).

### Example 3.3

### Preparation of 4-t-butylphenyl phenyl ether

Example 3.1 was repeated, replacing the phenol with 300 mg of 4-t-butylphenol (2 mmoles).

The degree of transformation and the selectivity for 4-t-butylphenyl phenyl ether were 100%.

The oily residue obtained after treatment was complete was purified by silica gel chromatography (eluent: hexane).

430 mg of a colourless oil was obtained (which corresponded to a yield of 95%), which crystallised after a few hours in the refrigerator (colourless crystals).

The compound obtained had the following formula:

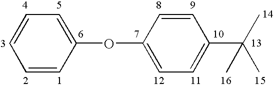

The characteristics were as follows:

M.Pt: 52° C. (Lit: 53-54° C., given Harvey, L; Gleicher, G J; To therow, W D; Tetrahedron 1969, 25, 5019-5026);

¹H NMR/DMSO-d₆: δ 7.33-7.41 (m, 4H, H_{2,8,12}), 7.06-7.14 (m, 1H, H₃), 6.91-6.99 (m, 4H, H_{1,5,9,11}), 1.27 (s, 9H, H_{14,15,16});

¹³C NMR/DMSO-d₆: δ 156.94 (C6), 154.09 (C7), 145.73 (C10), 129.88 (C2 and C4), 126.61 (C9 and C11), 123.05 (C3), 118.21 (C1, C5, C8 and C12), 33.96 (C13), 31.18 (C14, C15 and C16).

GC/MS: Rt=18.50 min, M/Z=226, purity=98.5%;

Rf: 0.36 (eluent: hexane).

### Example 3.4

### Preparation of 3,5-dimethylphenyl phenyl ether

Example 3.1 was repeated, replacing the phenol with 244 mg of 3,5-dimethylphenol (2 mmoles).

The degree of transformation and the selectivity for 3,5-dimethylphenyl phenyl ether were 100%.

The brown oil obtained after treatment was complete was purified by silica gel chromatography (eluent: hexane).

381 mg of a colourless oil was obtained, which corresponded to a yield of 97%.

The compound obtained had the following formula:

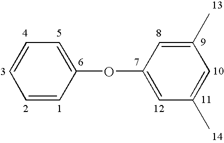

The characteristics were as follows:

¹H NMR/CDCl₃: δ 7.28-7.42 (m, 2H, H_{2,4}), 7.12-7.17 (m, 1H, H₃), 7.03-7.14 (m, 2H, H_{1,5}), 6.79 (m, 1H, H₁₀), 6.69 (m, 2H, H_{8,12}), 2.33 (s, 6H, H_{13,14});

¹³C NMR/CDCl₃: δ 157.50 (C6), 157.22 (C7), 139.61 (C9 and C11), 129.70 (C2 and C4), 125.04 (C10), 123.02 (C3), 118.89 (C1 and C5), 116.67 (C8 and C12), 21.35 (C13);

GC/MS: Rt=16.87 min, M/Z=198, purity=98%;

Rf: 0.19 (eluent: hexane).

### Example 3.5

### Preparation of 3,5-dimethylphenyl phenyl ether from bromobenzene

Example 3.3 was repeated, replacing the iodobenzene with bromobenzene (316 μl, 3 mmoles), the acetonitrile with DMF, and heating for 24 h at 110° C.

The degree of transformation of 3,5-dimethylphenyl ether was 70%.

The degree of transformation of 3,5-dimethylphenyl ether was 100% after heating for 72 h under these conditions.

### Example 3.6

### Preparation of 3,5-dimethylphenyl 4-trifluoromethylphenyl ether

Example 3.1 was repeated, replacing the phenol with 244 mg of 3,5-dimethylphenol (2 mmoles) and the iodobenzene with 294 μl of 4-iodotrifluoromethylbenzene (2.6 mmoles).

The degree of transformation and the selectivity for 3,5-dimethylphenyl 4-trifluoromethylphenyl ether were 100%.

The residue obtained after treatment was complete was purified by silica gel chromatography (eluent: hexane).

506 mg of an orange oil was obtained, which corresponded to a yield of 95%.

The compound obtained had the following formula:

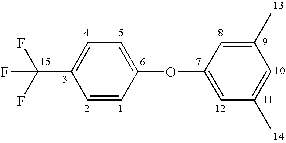

The characteristics were as follows:

¹H NMR/CDCl₃: δ 7.59 (m, 2H, H_{2,4}), 7.06 (m, 2H, H_{1,5}), 6.87 (m, 1H, H₁₀), 6.71 (m, 2H, H_{8,12}), 2.35 (s, 6H, H_{13,14});

¹³C NMR/CDCl₃: δ 160.78 (C6), 155.65 (C7), 140.01 (C9 and C11), 127.04 (q, ³J_{CF}=3.8 Hz, C2 and C4), 126.25 (C10), 124.59 (q, ²J_{CF}=32.7 Hz, C3), 118.92 (q, ¹J_{CF}=271.1 Hz, C15), 117.78 (C8 and C12), 117.63 (C1 and C5), 21.26 (C13 and C14);

¹⁹F NMR/CDCl₃: δ −2.11 (CF₃);

Elemental analysis: Calculated: C, 67.66%; H, 4.92%; F: 21.41%. Found: C: 67.37%; H, 5.03%; F: 21.80%;

GC/MS: Rt=16.71 min, M/Z=266, purity=99%;

IR (CH₂Cl₂): 3053 (VW, aromatic), 2985 (VW), 1615, 1591 and 1513 (W, aromatic C═C), 1326 (VS, CF₃), 1237 (S, C—O), 1169 (S, CF₃), 1123 (S), 1066 (S), 840 (W), 748 (VS), 730 (S).

Rf: 0.68 (eluent: hexane).

### Example 3.7

### Preparation of 3,5-dimethylphenyl 2-methylphenyl ether

Example 3.1 was repeated, replacing the phenol with 244 mg of 3,5-dimethylphenol (2 mmoles) and the iodobenzene with 383 ∥l of 2-iodotoluene (3 mmoles), and taking the reaction time to 118 hours.

The degree of transformation and the selectivity for 3,5-dimethylphenyl 2-methylphenyl ether were 100%.

The oily residue obtained after treatment was complete was purified by silica gel chromatography (eluent: hexane).

399 mg of a colurless oil was obtained, which corresponded to a yield of 94%.

The compound obtained had the following formula:

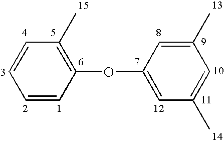

The characteristics were as follows:

¹H NMR/CDCl₃: δ 7.08-7.33 (m, 3H, H_{2,3,4}), 6.95-6.99 (m, 1H, H₁), 6.76 (m, 1H, H₁₀), 6.61 (m, 2H, H_{8,12}), 2.33 (s, 6H, H_{13,14}), 2.32 (s, 3H, H₁₅) [Buchwald, S L; Marcoux, J-F; Doye, S; J. Am. Chem. Soc. 1997, 119, 10539-10540, Supporting Information);

¹³C NMR/CDCl₃: δ 157.94 (C6), 154.69 (C7), 139.55 (C9 and C11), 131.41 (C2), 130.02 (C5), 127.14 (C4), 124.22 (C10), 123.83 (C3), 119.81 (C1), 115.11 (C8 and C12), 21.42 (C13 and C14), 16.30 (C15);

GC/MS: Rt=17.46 min, M/Z=212, purity=99.7%;

Rf: 0.26 (eluent: hexane).

### Example 3.8

### Preparation of 3,5-dimethylphenyl 4-methoxyphenyl ether

Example 3.1 was repeated, replacing the phenol with 244 mg of 3,5-dimethylphenol (2 mmoles) and the iodobenzene with 655 mg of 4-iodoanisole (2.8 mmoles), the latter being added at the same time as the 3,5-dimethylphenol, and taking the reaction time to 48 hours.

The degree of transformation and the selectivity for 3,5-dimethylphenyl 4-methoxyphenyl ether were 100%.

The residue obtained after treatment was placed in an oven at 100° C. to evaporate off the anisole, then it was purified by silica gel chromatography (eluent: hexane).

420 mg of a colourless oil was obtained, which corresponded to a yield of 92%.

Crystals could be obtained after re-crystallisation from petroleum ether.

The compound obtained had the following formula:

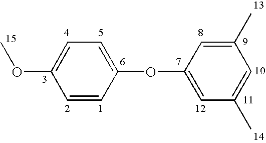

The characteristics were as follows:

M.Pt: 67° C. (Lit: 67° C., given by Walter; Barell-Festschr., Basel 1936, 266-273);

¹H NMR/CDCl₃: δ 6.99-7.06 (m, 2H, H_{2,4}), 6.88-6.99 (m, 2H, H_{1,5}), 6.74 (m, 1H, H₁₀), 6.64 (m, 2H, H_{8,12}), 3.85 (s, 3H, H₁₅), 2.32 (s, 6H, H_{13,14});

¹³C NMR/CDCl₃: δ 158.52 (C3), 155.76 (C7), 150.26 (C6), 139.45 (C9 and C11), 124.22 (C10), 120.84 (C1 and C5), 115.31 (C2 and C4), 114.77 (C8 and C12), 55.59 (C15), 21.35 (C13 and C14);

GC/MS: Rt=19.77 min, M/Z=228, purity=99%;

Rf: 0.61 (eluent: hexane).

### Example 3.9

### Preparation of 3,5-dimethylphenyl 4-cyanophenyl ether

Example 3.1 was repeated, replacing the phenol with 244 mg of 3,5-dimethylphenol (2 mmoles) and the iodobenzene with 595 mg of 4-iodobenzonitrile (2.6 mmoles), the latter being added at the same time as the 3,5-dimethylphenol.

The degree of transformation and the selectivity for 3,5-dimethylphenyl 4-cyanophenyl ether were 100%.

The residue obtained after treatment was placed in an oven at 100° C. to evaporate off the benzonitrile then it was purified by silica gel chromatography (eluent: hexane/dichloromethane, 100/0 to 50/50).

415 mg of an orange solid was obtained, which corresponded to a yield of 93%.

The compound obtained had the following formula:

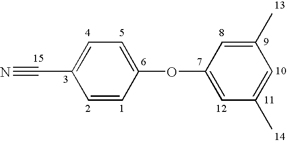

The characteristics were as follows:

M.Pt: 58° C.;

¹H NMR/CDCl₃: δ 7.53-7.60 (m, 2H, H_{2,4}), 6.95-7.00 (m, 2H, H_{1,5}), 6.86 (m, 1H, H₁₀), 6.68 (m, 2H, H_{8,12}), 2.32 (s, 6H, H_{13,14});

¹³C NMR/CDCl₃: δ 161.90 (C6), 154.76 (C7), 140.17 (C9 and C11), 134.07 (C2 and C4), 126.86 (C10), 118.92 (C15), 118.03 (C1 and C5), 117.88 (C8 and C12), 105.55 (C3), 21.28 (C13 and C14);

GC/MS: Rt=20.54 min, M/Z=223, purity=100%;

Rf: 0.32 (eluent: hexane/dichloromethane, 50/50).

### Example 3.10

### Preparation of bis(o-tolyl) ether

Example 3.1 was repeated, replacing the phenol with 206 μl of o-cresol (2 mmoles) and the iodobenzene with 383 μl of 2-iodotoluene (3 mmoles), the acetonitrile with DMF, and with the nucleophile and the arylation agent being added at the same time as the solvent.

The reaction time was taken to 35 h and the temperature was 110° C.

The degree of transformation and the selectivity for bis(o-tolyl) ether were 100%.

The oily residue obtained after treatment purified by silica gel chromatography (eluent: hexane).

389 mg of a colourless oil was obtained, which corresponded to a yield of 98%.

The compound obtained had the following formula:

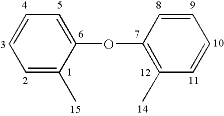

The characteristics were as follows:

¹H NMR/CDCl₃: δ 7.32 (m, 2H, H_{2,11}), 7.04-7.25 (m, 4H, H_{3,4,9,10}), 6.81 (m, 2H, H_{5,8}), 2.38 (s, 6H, H_{13,14});

¹³C NMR/CDCl₃: δ 155.35 (C6 and C7), 131.39 (C4 and C9), 128.90 (C1 and C12), 127.09 (C2 and C11), 123.11 (C3 and C10), 117.74 (C5 and C8), 16.25 (C13 and C14);

GC/MS: Rt=16.10 min, M/Z=198, purity=100%;

Rf: 0.40 (eluent: hexane).

### Example 3.11

### Preparation of phenyl 2-methylphenyl ether

Example 3.1 was repeated, replacing the phenol with 206 μl of o-cresol (2 mmoles), and with the nucleophile and the arylation agent being added at the same time as the solvent.

The reaction time was taken to 40 h.

The degree of transformation and the selectivity for phenyl 2-methylphenyl ether were 100%.

The oily residue obtained after treatment was purified by silica gel chromatography (eluent: hexane).

343 mg of a colourless oil was obtained, which corresponded to a yield of 93%.

The compound obtained had the following formula:

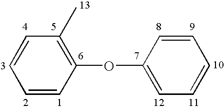

The characteristics were as follows:

¹H NMR/CDCl₃: δ 7.19-7.35 (m, 3H, H_{4,9,11}), 7.00-7.18 (m, 3H, H_{2,3,10}), 6.87-6.94 (m, 3H, H_{1,8,12}), 2.25 (s, 3H, H₁₃);

³C NMR/CDCl₃: δ 158.08 (C7), 54.60 (C6), 131.60 (C2), 130.14 (C5), 129.81 (C9 and C11), 127.30 (C4), 124.15 (C10), 122.48 (C3), 119.94 (C1), 117.44 (C8 and C12), 16.35 (C13);

GC/MS: Rt=15.25 min, M/Z=184, purity=98%;

Rf: 0.36 (eluent: hexane).

### Example 3.12

### Preparation of 3,5-dimethylphenyl 2-pyridyl ether

General procedure A (110° C., 24 hours) was followed using 117 mg of Chxn-Py-Al (0.4 mmoles), 292 μl of 2-bromopyridine (3 mmoles), 244 mg of 3,5-dimethylphenol (2 mmoles), 600 mg of ground and activated 3 Å molecular sieve and 1.2 ml of DMF.

The oil obtained after the filtering step was oven dried for two hours at 100° C. to evaporate off the 2-pyridylaldehyde then purified by silica gel chromatography (eluent: hexane/dichloromethane, 100/0 to 85/15).

371 mg of a yellow oil was obtained, which corresponded to a yield of 93%.

The compound obtained had the following formula:

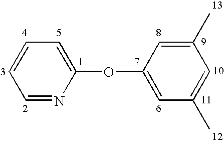

The characteristics were as follows:

¹H NMR/CDCl₃: δ 8.21 (ddd, 1H, ³J_{HH}=5.0 Hz, ⁴J_{HH}=2.0 Hz, ⁵J_{HH}=0.7 Hz, H₂), 7.66 (ddd, 1H, ³J_{HH}=8.2 Hz, ³J_{HH}=7.2 Hz, ⁴J_{HH}=2.0 Hz, H₄), 6.97 (ddd, 1H, ³J_{HH}=7.2 Hz, ³J_{HH}=5.0 Hz, ⁴J_{HH}=0.9 Hz, H₃), 6.88 (ddd, 1H, ³J_{HH}=8.2 Hz, ⁴J_{HH}=0.9 Hz, ⁵J_{HH}=0.7 Hz, H₅), 6.84 (m, 1H, H₁₀), 6.76 (m, 2H, H_{6,8}), 2.32 (s, 6H, H_{12,13});

¹³C NMR/CDCl₃: δ 164.02 (C1), 154.15 (C7), 147.87 (C2), 139.47 (C9 and C11), 139.27 (C4), 126.53 (C10), 118.80 (C6 and C8), 118.22 (C5), 111.47 (C3), 21.34 (C12 and C13);

Elemental analysis: Calculated: C, 78.21%; H, 6.69%; N, 7.04%. Found C, 78.36%;

H, 6.58%; N, 7.03%;

GC/MS: Rt=17.65 min, M/Z=199, purity=99%;

IR (CH₂Cl₂): 3027 (VW, aromatic), 1468 and 1430 (VW, aromatic C═C), 1220 (S, C—O), 781 (S), 759 (VS), 751 (S);

Rf: 0.22 (eluent: hexane/dichloromethane, 75/25).

### Example 4

### Arylation of Carbon-Containing Nucleophiles

### Example 4.1

### Synthesis of diethyl 2-phenylmalonate

38 mg of cuprous iodide (0.2 mmoles), 117 mg of Chxn-Py-A1 (0.4 mmoles) and 977 mg of caesium carbonate (3 mmoles) were successively introduced into a 35 ml Schlenk tube that had been oven dried at 100° C. and provided with a magnetic stirrer (12×4.5 mm) and under a nitrogen atmosphere.

The Schlenk tube was purged under vacuum then refilled with nitrogen.

607 μl of diethyl malonate (3 mmoles), 224 μl of iodobenzene (2 mmoles), 1.2 ml of acetonitrile and 600 mg of ground and activated 3 Å molecular sieve were then added.

The reactor was placed in an oil bath at a temperature of 70° C. and stirred for a period of 30 hours.

The reaction mixture was neutralized with 6 ml of an aqueous 1N hydrochloric acid solution before being filtered through celite.

The filtrate was extracted with dichloromethane then concentrated under reduced pressure.

The residue obtained was purified directly by silica column chromatography (eluent: hexane/dichloromethane, 100/0 to 80/20).

439 mg of colourless oil was obtained, corresponding to a yield of 93%.

A compound with the following formula was obtained:

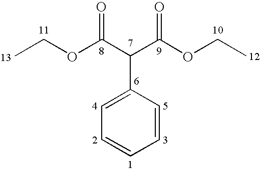

The characteristics were as follows:

¹H NMR/CDCl₃: δ 7.32-7.42 (m, 5H, H₁₋₅), 4.62 (s, 1H, H₇), 4.22 (m, 4H, H_{10,11}), 1.26 (t, ³J_{HH}=7.1 Hz, 6H, H_{12,13}). The protons of each methylene fragment and the ester function were diastereotopic and resulted in a second order mass.

¹³C NMR/CDCl₃: δ 168.15 (C8 and C9), 132.86 (C6), 129.27 (C2 and C3), 128.58 (C4 and C5), 128.18 (C1), 61.77 (C11 and C12), 58.00 (C7), 14.00 (C12 and C13);

GC/MS: Rt=16.77 min, M/Z=236, purity=99%;

Rf: 0.27 (eluent: hexane/dichloromethane, 70/30).

### Example 4.2

### Synthesis of ethyl 2-phenylcyanoacetate

117 mg of Chxn-Py-Al (0.4 mmoles) and 977 mg of caesium carbonate (3 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen.

427 μl of ethyl cyanoacetate (4 mmole), 224 μl of iodobenzene (2 mmole), 1.2 ml of acetonitrile and 600 mg of 3 Å ground molecular sieve were then added.

The reactor was placed in an oil bath at a temperature of 70° C. and stirred for 28 hours.

The reaction mixture was neutralized with 6 ml of an aqueous 1N hydrochloric acid solution before being filtered through celite.

The filtrate was extracted with dichloromethane then concentrated under reduced pressure.

The residue obtained was directly purified by silica column chromatography (eluent: dichloromethane/hexane 100/0 to 75/25).

348 mg of colourless oil was obtained which corresponded to a yield of 92%.

A compound was obtained with the following formula: 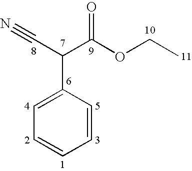
with the following characteristics:

¹H NMR/CDCl₃: δ 7.37-7.45 (m, 5H, H₁₋₅), 4.71 (s, 1H, H₇), 4.25 (q, 2H, ³J_{HH}=7.1 Hz, H₁₀), 1.28 (t, 3H, ³J_{HH}=7.1 Hz, H₁₁).

¹³C NMR/CDCl₃: δ 164.99 (Cg), 130.04 (C₆), 129.33 (C₂ and C₃), 129.21 (C1), 127.91 (C₄₋₅), 115.66 (C₈), 63.28 (C10), 43.74 (C₇), 13.87 (C11).

GC/MS: Rt=15.24 min, M/Z=189, purity=99%;

Rf: 0.22 (eluent: hexane/dichloromethane, 75/25).

### Example 4.3

### Synthesis of 2-phenylmalononitrile

38 mg (0.2 mmoles) of cuprous iodide, 117 mg of Chxn-Py-Al (0.4 mmoles) and 977 mg of caesium carbonate (3 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen.

132 mg of malonitrile (4 mmole), 224 μl of iodobenzene (2 mmole), 1.2 ml of acetonitrile and 600 mg of 3 Å ground and activated molecular sieve were then added.

The reactor was placed in an oil bath at a temperature of 50° C. and stirred for 72 hours.

The reaction mixture was neutralized with 6 ml of an aqueous 1N hydrochloric acid solution before being filtered through celite.

The filtrate was extracted with dichloromethane then concentrated under reduced pressure.

The black residue obtained was directly purified by silica column chromatography (eluent: dichloromethane/hexane 100/0 to 60/40) 176 mg of colourless solid was obtained, corresponding to a yield of 62%.

A compound was obtained with the following formula: 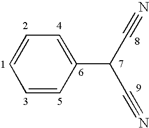
with the following characteristics:

M Pt: 64-65° C. (lit: 66-68° C., hexane/diethyl ether)

¹H NMR/CDCl₃: δ 7.51 (m, 5H, H_{1,5}), 5.08 (s, 1H, H₇)

¹³C NMR/CDCl₃: δ 130.40 (C₆), 130.06 (C₂ and C₃), 127.22 (C1), 126.23 (C₄₋₅), 111.77 (C₈₋₉), 28.10 (C₇).

GC/MS: Rt=12.96 min, M/Z=189, purity=99%;

Rf: 0.32 (eluent: hexane/dichloromethane, 50/50).

### Example 4.4

Synthesis of Benzonitrile

3.6 mg of copper I oxide (0.025 mmoles), 29.2 mg of Chxn-Py-Al (0.1 mmoles) and 35.8 mg of KCN (0.55 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen. 56 μl of iodobenzene (0.5 mmole) and 300 μl of anhydrous DMF were then added.

The reactor was placed in an oil bath at a temperature of 110° C. and stirred for 24 hours.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The benzonitrile yield was 73.7% and the selectivity was 96%.

### Example 4.5

Synthesis of Benzonitrile

3.6 mg of copper I oxide (0.025 mmoles), 29.2 mg of Chxn-Py-Al (0.1 mmoles) and 35.8 mg of KCN (0.55 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen.

56 μl of iodobenzene (0.5 mmole) and 300 μl of anhydrous DMF were then added.

The reactor was placed in an oil bath at a temperature of 110° C. and stirred for 48 hours.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The benzonitrile yield was 86.9% and the selectivity was 94%.

### Example 4.7

Synthesis of Benzonitrile

3.6 mg of copper I oxide (0.025 mmoles), 17.7 mg of DAPAE (0.1 mmoles) and 35.8 mg of KCN (0.55 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen. 56 μl of iodobenzene (0.5 mmole) and 300 μl of anhydrous DMF were then added.

The reactor was placed in an oil bath at a temperature of 110° C. and stirred for 24 hours.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The benzonitrile yield was 83.0% and the selectivity was 97.5%.

### Example 4.8

Synthesis of Benzonitrile

3.6 mg of copper I oxide (0.025 mmoles), 17.7 mg of DAPAE (0.1 mmoles) and 41.5 mg of KI (0.25 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen. 53 μl of bromoobenzene (0.5 mmole) and 300 μl of anhydrous DMF were then added.

At the end of this period, 37.6 mg of KCN (0.58 mmole) was added to the cooled reaction medium all at once. The reactor was reheated to a temperature of 110° C. and stirred for a period of 24 hours.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The benzonitrile yield was 30.2% and the selectivity was 100%.

### Example 4.9

Synthesis of Benzonitrile

3.6 mg of copper I oxide (0.025 mmoles), 17.7 mg of DAPAE (0.1 mmoles) and 41.5 mg of KI (0.25 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen. 53 μl of bromoobenzene (0.5 mmole) and 300 μL of anhydrous DMF were then added.

The reactor was placed in an oil bath at a temperature of 110° C. and stirred for a period of 23 hours.

At the end of this period, 11.9 mg of KCN (0.18 mmole) was added to the cooled reaction medium all at once. The reactor was reheated to a temperature of 110° C. 17 hours later, a new addition of 27.3 mg of KCN was made under the same conditions as before (41 mmoles).

The mixture was kept at 110° C. for 7 h.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The benzonitrile yield was 36.1% and the selectivity was 100%.

### Example 5

### Arylation of Other Nitrogen-Containing Nucleophiles: Amines

### Example 5.1

Synthesis of Triphenylamine

9.5 mg of copper I iodide (0.050 mmoles), 29.2 mg of DAPAE (0.1 mmoles, 127 mg of Ph₂NH (0.75 mmole) and 325.8 mg of caesium carbonate (1 mmole) were successively introduced into a 35 ml Schlenk tube which had been oven dried at 100° C. provided with a magnetic stirrer (12×4.5 mm) and placed in a nitrogen atmosphere.

The Schlenk tube was purged under vacuum then refilled with nitrogen. 56 μl of iodoobenzene (0.5 mmole) and 300 μl of anhydrous toluene were then added.

The reactor was placed in an oil bath at a temperature of 110° C. and stirred for a period of 24 hours.

After this period, 65 μl of 1,3-dimethoxybenzene (internal standard) was introduced into the cooled reaction medium which was then diluted with 5 ml of diethyl ether.

An aliquot was removed, filtered over celite eluting with ethyl ether, extracted three times with distilled water then analyzed by gas chromatography.

The triphenylamine yield was 53.1% and the selectivity was 100%.

1-61. (canceled)

62. The process process for creating a carbon-carbon or carbon-heteroatom bond by reacting an unsaturated compound carrying a leaving group and a nucleophilic compound carrying a carbon atom or a heteroatom (HE) that can substitute for the leaving group, thus creating a C—C or C-HE bond, wherein the reaction takes place in the presence of an effective quantity of a copper-based catalyst and at least one ligand comprising at least one imine function and at least one supplemental nitrogen atom as chelating atoms.

63. The process according to claim 62, wherein the ligand is bidentate, tridentate or tetradentate in type.

64. The process according to claim 63, wherein the ligand employed has the following formulae: 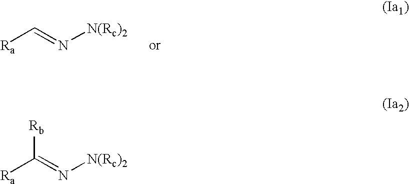
in which formulae:
one of groups Rₐ and R_{b} comprises at least one nitrogen atom or a group comprising a nitrogen atom;
Rₐ and R_{b} independently represent a hydrocarbon group containing 1 to 20 carbon atoms, which may be a linear or branched, saturated or unsaturated, acyclic aliphatic group; a monocyclic or polycyclic, saturated, unsaturated or aromatic carbocyclic or heterocyclic group; or a concatenation of said groups; or
Rₐ and R_{b} are be bonded to constitute, with the carbon atoms carrying them, a monocyclic or polycyclic, saturated or unsaturated carbocyclic or heterocyclic group containing 3 to 20 atoms;
at most one of groups Rₐ and R_{b} represents a hydrogen atom; and
R_{c}, which may be identical or different, represents a hydrogen atom, an alkyl group, preferably C₁ to C₁₂; an alkenyl or alkynyl group, preferably C₂ to C₁₂; a cycloalkyl group, preferably C₃ to C₁₂; an aryl or arylalkyl group, preferably C₆ to C₁₂, an amido group —CO—NH₂; an amido group substituted with one or two alkyl groups, preferably C₁ to C₁₂; and/or an alkenyl or alkynyl group, preferably C₂ to C₁₂; and/or a cycloalkyl group, preferably C₃ to C₁₂; and/or an aryl or arylalkyl group, preferably C₆ to C₁₂.

65. The process according to claim 64, wherein the ligand has formula (Ia₁) or (Ia₂) in which groups R_{c} represent a hydrogen atom or a C₁-C₄ alkyl group, an amido group, or an amido group substituted with a C₁-C₄ alkyl group.

66. The process according to claim 64, wherein the ligand has formula (Ia₁) or (Ia2) in which groups Rₐ and R_{b} represent one of the groups with formula (F₀): 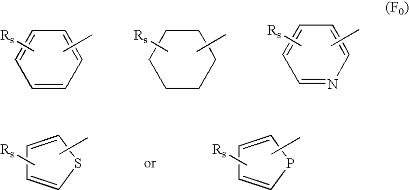
in which R_{S} represents a hydrogen atom, an alkyl or alkoxy group, preferably C₁ to C₄, or an amino or amido group optionally substituted with an alkyl group, or a phosphino group substituted with alkyl groups, identical or different, or with phenyl groups.

67. The process according to claim 64, wherein the ligand has formula (Ia1) or (Ia2) in which groups Rₐ and R_{b} represent one of the groups with formula (F₄): 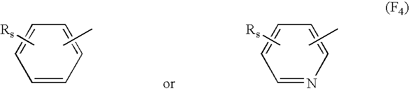
in which R_{S} represents a hydrogen atom, an alkyl or alkoxy group, or an amino or amido group optionally substituted with an alkyl group.

68. The process according to claim 64, wherein the ligand has formula (Ia1) in which groups R_{c}, which are identical or different, represent a hydrogen atom or a methyl group, and Rₐ represents one of the groups with formula (F₄).

69. The process according to claim 63, wherein the ligand has the following formulae: 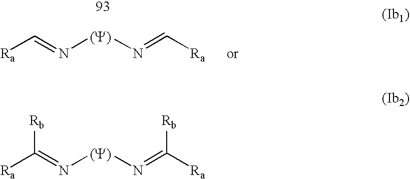
in which formulae:
Rₐ, which are identical or different, have the meanings given in formulae (Ia₁) and (Ia₂);
R_{b}, which are identical or different, have the meanings given in formulae (Ia₁) and (Ia₂);
Rₐ and/or R_{b} represent a hydrogen atom;
Ψ represents a covalent bond, a urea group or a skeleton with general formula (F₂) or (F₃): 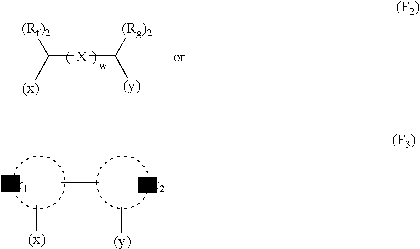
in which formulae (F₂) and (F₃):
R_{f} and R_{g}, which are identical or different, independently represent a hydrogen atom, a hydrocarbon group containing 1 to 20 carbon atoms, which are a linear or branched, saturated or unsaturated acyclic aliphatic group; a monocyclic or polycyclic, saturated, unsaturated or aromatic carbocyclic or heterocyclic group; or a concatenation of said groups; or
R_{f} and R₉ are bonded together to constitute, with the carbon atoms carrying them, a carbocyclic or heterocyclic group containing 3 to 20 atoms, which are saturated, unsaturated, monocyclic or polycyclic;
Ar₁ and Ar₂ independently represent two substituted or non substituted aromatic, carbocyclic or heterocyclic cycles optionally condensed, and having one or more heteroatoms;
X represents a methylene group optionally substituted;
w is a whole number from 0 to 3; and
x and y respectively represent the two bonds between the skeleton shown as ψ and the imine groups.

70. The process according to claim 69, wherein the ligand has formulae (Ib₁) or (Ib₂) in which groups Rₐ and R_{b} represent one of the following groups: 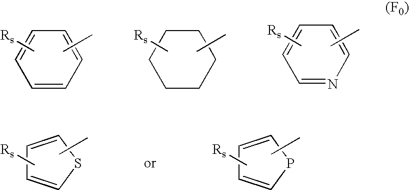
wherein R_{S} represents a hydrogen atom, an alkyl group, an alkoxy group, preferably C₁ to C₄, or an amino or amido group optionally substituted with alkyl groups, or a phosphino group substituted with alkyl groups, or phenyl groups.

71. The process according to claim 70, wherein the ligand has formula (Ib1) or (Ib2) in which groups Rₐ and R_{b} represent one of the groups with formula (F₅): 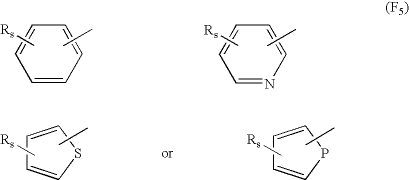
wherein R_{S} represents a hydrogen atom, an alkyl or alkoxy group, or an amino or amido group optionally substituted with alkyl groups.

72. The process according to claim 69, wherein the ligand has formula (Ib₁) or (Ib₂), in which Ψ represents a covalent bond, a urea group or one of the following cyclic groups:

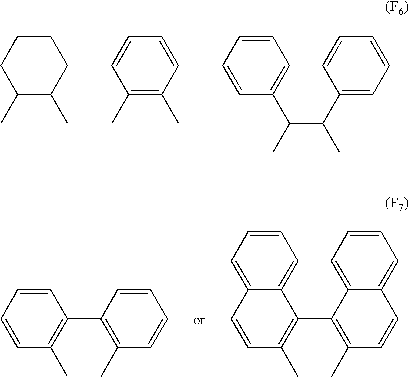

73. The process according to claim 69, wherein the ligand has formula (Ib1) in which the group Rₐ represents one of the groups with formula (F₅) and Ψ represents a covalent bond, a urea group or one of groups (F₆) and (F₇).

74. The process according to claim 69, wherein the ligand has the following formula: 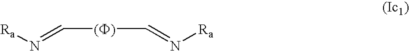
in which formula:
Rₐ, which may be identical or different, have the meanings given in formulae (Ia₁) and (Ia₂);
Φ represents:
a covalent bond;
an alkylene group with formula: 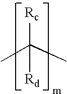
in which R_{c}, R_{d}, which are identical or different, represent:
a hydrogen atom;
a linear or branched alkyl group containing 1 to 12 carbon atoms, optionally carrying a halogen atom, preferably 1 to 4 carbon atoms, such as methyl, ethyl, propyl, isopropyl, butyl, isobutyl, sec-butyl or tert-butyl;
a halogen atom; and
m equals 0, 1 or 2, preferably 0 or 1; or
or the residue of a saturated, unsaturated or aromatic, monocyclic or polycyclic hydrocarbon cycle containing 5 to 12 carbon atoms carrying the two imine functions in the ortho or meta position.

76. The process according to claim 75, wherein the ligand has formula (Ic₁) in which groups Rₐ represent one of the following groups (F₀): 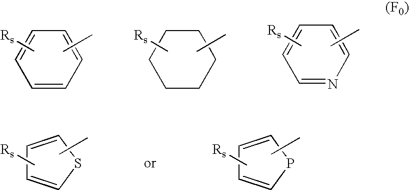
in which R_{S} represents a hydrogen atom, an alkyl or alkoxy group, or an amino or amido group optionally substituted with alkyl groups, or a phosphino group substituted with alkyl groups, or phenyl groups, optionally identical.

77. The process according to claim 75, wherein the ligand has formula (Ic1) in which groups Rₐ represent one of the following groups with formula (F₈): 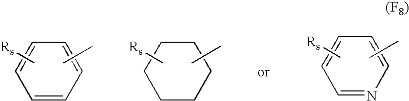
in which R_{S} represents a hydrogen atom, an alkyl or alkoxy group, or an amino group optionally substituted with an alkyl group.

78. The process according to claim 75, wherein the ligand has formula (Ic₁) in which Φ represents a covalent bond, a methylene or ethylene group, or a divalent cyclic group having the formula:

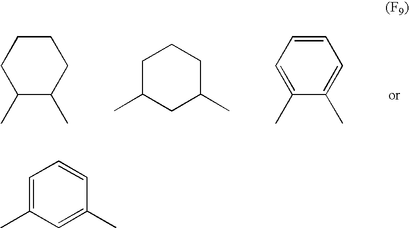

79. The process according to claim 75, wherein the ligand has formula (Ic1) in which φ represents a covalent bond, a methylene or ethylene group, one of groups (F₉) and groups Rₐ, optionally identical, representing one of the groups with formula (F₈).

80. The process according to claim 63, wherein the ligand has the following formulae: 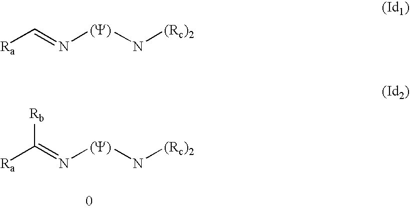
in which formulae:
Rₐ, which are identical or different, have the meanings given in formulae (Ia₁) and (Ia₂);
R_{b}, which are identical or different, have the meanings given in formulae (Ia₁) and (Ia₂);
Rₐ and/or R_{b} represent a hydrogen atom;
R_{c}, which are identical or different, have the meanings given in formulae (Ia₁) and (Ia₂); at most one of groups R_{c} represents a hydrogen atom; and
ψ represents a covalent bond or a skeleton with general formula (F₂) or (F₃(as defined in formulae (1B₁) and (1b₂).

81. The process according to claim 80, wherein the ligand has formula (Id1) or (Id2) in which groups Rₐ and R_{b} represent one of the groups with formula (F₀): 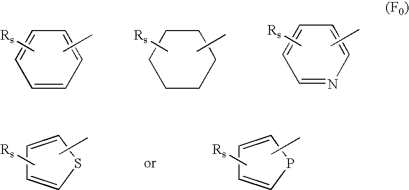
wherein R_{S} represents a hydrogen atom, an alkyl or alkoxy group, or an amino or amido group optionally substituted with alkyl groups, or a phosphino group substituted with alkyl groups or phenyl groups, optionally identical.

82. The process according to claim 80, wherein the ligand has formula (Id1) or Id2) in which groups Rₐ and R_{b} represent one of the groups with formula (F₁₀): 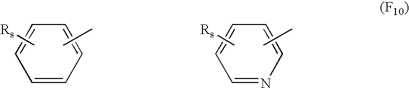
wherein R_{S} represents a hydrogen atom, an alkyl or alkoxy group or an amino group optionally substituted with an alkyl group.

83. The process according to claim 80, wherein the ligand has formula (Id1) or (Id2) in which groups R_{c}, which are identical or different, represent an alkyl group containing 1 to 4 carbon atoms.

84. The process according to claim 80, wherein the ligand has formula (Id1) or (Id2) in which the group ψ represents a methylene or ethylene group.

85. The process according to claim 80, wherein the ligand has formula (Id₁) in which group Rₐ represents one of the groups with formula (F₁₀), groups R_{c}, which are identical or different, represent an alkyl group containing 1 to 4 carbon atoms, and the group ψ represents a methylene or ethylene group.

86. The process according to claim 62, wherein the ligand is: Ph-Alzone, Py-Alzone, N-Methyl-Py-Alzone, N-Dimethyl-Py-Alzone, N-Amido-Py-Alzone, Chxn-Phenyl-Al, Chxn-Py-Al, Carbo-Py-Al, Chxn-Thio-Al, DAB-Cy.

87. The process according to claim 62, wherein the ligand is employed in a quantity with a ratio between the number of moles of ligand and the number of moles of copper being in the range 20 to 0.9.

88. The process according to claim 62, wherein the nucleophilic substrate is an organic hydrocarbon compound, acyclic or cyclic having at least one atom carrying a free electron pair optionally carrying a charge, or having a carbon atom able to donate its electron pair.

89. The process according to claim 88, wherein the nucleophilic substrate comprises at least one of the following atoms or groups:

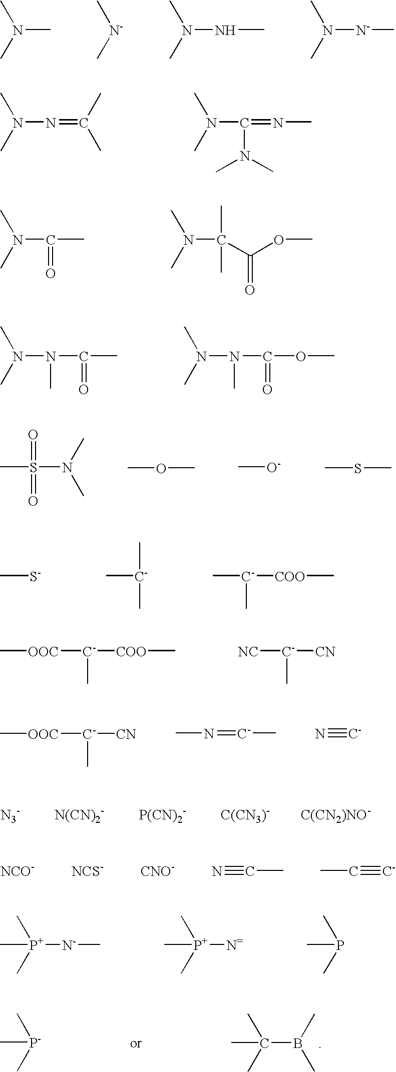

90. The process according to claim 62, wherein the nucleophilic substrate comprises at least one nitrogen atom carrying a free electron pair included in a saturated, unsaturated or aromatic cycle: the cycle generally comprising 3 to 8 atoms.

91. The process according to claim 90, wherein the nucleophilic substrate is a primary or secondary amine; a hydrazine or hydrazone derivative; an amide; a sulphoamide; a urea derivative; or a heterocyclic derivative, optionally nitrogen- and/or sulphur-containing.

92. The process according to claim 90, wherein the nucleophilic substrate has the following formula: 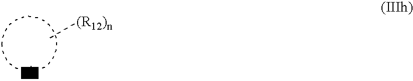
in which formula (IIIh):
A represents the residue of a cycle forming all or a portion of a monocyclic or polycyclic, aromatic or non aromatic heterocyclic system wherein one of the carbon atoms is replaced by at least one nucleophilic;
R₁₂, which are identical or different, represent substituents on the cycle; and
n represents the number of substituents on the cycle.

93. The process according to claim 92 wherein the nucleophilic substrate has formula (IIIh) in which A represents: imidazole, pyrazole, triazole, pyrazine, oxadiazole, oxazole, tetrazole, indole, pyrole, phthalazine, pyridazine or oxazolidine.

94. The process according to claim 88, wherein the nucleophilic substrate is an alcohol or thiol type compound, preferably a □ydroxy]- or thioaromatic type compound.

95. The process according to claim 91, wherein the nucleophilic substrate has the following formula: 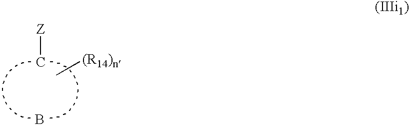
wherein:
B represents the residue of a monocyclic or polycyclic, aromatic carbocyclic group or a divalent group constituted by a concatenation of two or more monocyclic aromatic carbocyclic groups;
R₁₄ represents one or more substituents, which are identical or different;
Z represents a hydroxyl or thiol group; and
n′ is 5 or less.

96. The process according to claim 88, wherein the nucleophilic substrate is a hydrocarbon compound containing a nucleophilic carbon, or a nucleophilic compound comprising a carbanion in which the counter-ion is a metal.

97. The process according to claim 88, wherein the nucleophilic substrate is a phosphide, phosphine, phosphonium diazaylide, phosphonium azaylide, or boronic acid.

98. The process according to claim 88, wherein the nucleophilic substrate is a boronic acid with the following formula: 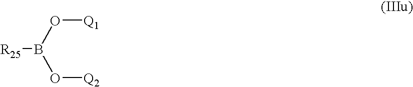
wherein:
R₂₅ represents a monocyclic or polycyclic, aromatic, carbocyclic or heterocyclic group; and
Q₁, Q₂, which are identical or different, represent a hydrogen atom, a linear or branched, saturated or unsaturated aliphatic group containing 1 to 20 carbon atoms, or a R₂₅ group.

99. The process according to claim 86, wherein the arylboronic acid R₂₄ has formula (IIIu) in which R₂₅ represents an aromatic carbocyclic or heterocyclic group, preferably a phenyl or naphthyl group, or a pyrrolyl, pyridyl, pyrimidyl, pyridazinyl, pyrazinyl, 1,3-thiazolyl, 1,3,4-thiadiazolyl or thienyl group.

100. The process according to claim 98, wherein the arylboronic acid has formula (IIIu) in which Q₁, Q₂, which may be identical or different, represent a hydrogen atom or a linear or branched acyclic aliphatic group containing 1 to 20 carbon atoms which may be saturated or contain one or more unsaturated bonds in the chain, preferably 1 to 3 unsaturated bonds, preferably simple or conjugated double bonds; or a group R₂₅, preferably a phenyl group.

101. The process according to one claim 62, wherein the nucleophilic compound is: pyrazole, 3,5-dimethylpyrazole, imidazole, indole, 1,2,4-triazole, pyrrole, 4-bromoaniline, 1-methyl-4-bromopyrazole, 3-trifluoromethyl-5-(p-tolyl)-1H-pyrazole, 5-(3-chlorosulphonyl-4-methylphenyl)-3-trifluoromethyl-1H-pyrazole, oxazolidin-2-one, 2-hydroxypyridine, benzamide, pyrrolidin-2-one, benzenesulphonamide, phenol, 4-methoxypheonl, 3,5-dimethyphenol, o-cresol, dimethyl malonate, ethyl malonate, methyl cyanoacetate, ethyl cyanoacetate, malonitrile, potassium cyanide, sodium cyanide or diphenylamine.

102. The process according to claim 62, wherein the compound carrying a leaving group Y is represented by formula (IV):
R₀—Y (IV)
wherein formula R₀ represents a hydrocarbon group containing 2 to 20 carbon atoms and has a double bond or a triple bond located in the position a to the leaving group Y, or a monocyclic or polycyclic, aromatic, carbocyclic and/or heterocyclic group.

103. The process according to claim 102, wherein the compound carrying a leaving group has formula (IV) wherein:
R₀ represents an aliphatic hydrocarbon group containing a double bond or a triple bond in the position a to the leaving group or a cyclic hydrocarbon group containing an unsaturated bond carrying a leaving group;
R₀ represents a monocyclic or polycyclic, aromatic, carbocyclic and/or heterocyclic group; and
Y represents a leaving group, optionally a halogen atom or a sulphonic ester group with formula —SO₂—Rₑ, in which Rₑ is a hydrocarbon group.

104. The process according to claim 102, wherein the compound carrying a leaving group has formula (IV) in which Y represents a bromine or chlorine atom or a sulphonic ester with formula —SO₂—Rₑ, in which Rₑ is a linear or branched alkyl group containing 1 to 4 carbon atoms or a trifluoromethyl group.

105. The process according to claim 102, wherein the compound carrying a leaving group has formula (IV) and is an:
(1) aliphatic type compound, carrying a double bond and having formula (IVa): 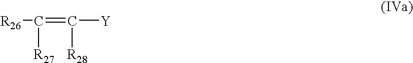
in which formula (IVa):
R₂₆, R₂₇ and R₂₈, which are identical or different, represent a hydrogen atom or a hydrocarbon group containing 1 to 20 carbon atoms, which linear or branched, saturated or unsaturated aliphatic group; a monocyclic or polycyclic, saturated, unsaturated or aromatic carbocyclic or heterocyclic group; or a concatenation of aliphatic and/or carbocyclic and/or heterocyclic groups as defined above;
Y represents the leaving group, as defined above;
(2) aliphatic compound carrying a triple bond, represented by formula (IVb):
R₂₆—C═C—Y (IVb)
whrein:
R₂₆ has the meaning given in formula (IVa); and
Y represents a leaving group as defined above; or
(3) aromatic compound having formula (IVc): 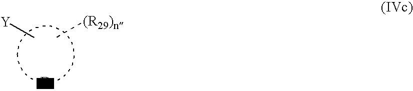
wherein:
D represents the residue of a cycle forming all or a portion of a monocyclic or polycyclic, aromatic, carbocyclic and/or heterocyclic system;
R₂₉, identical or different, represent substituents on the cycle;
Y represents a leaving group as defined above; and
n″ represents the number of substituents on the cycle.

106. The process according to claim 102, wherein the compound carrying a leaving group with formula (IV) is: vinyl chloride, vinyl bromide, bromoalkyne, iodoalkyne, β-bromostyrene, β-chlorostyrene, p-chlorotoluene, p-bromoanisole or p-bromotrifluorobenzene.

107. The process according to claim 102, wherein the compound carrying a leaving group with formula (IV) is: bromobenzene, 2-iodotoluene, 1,4-dibromobenzene, 1-(4′-bromophenyl)-1H-inmidazole, 1-(4′-bromophenyl)-1H-pyrazole, β-bromostyrene, iodobenzene, 4-bromotrifluoromethylbenzene, 4-iodotrifluoromethylbenzene, 4-iodoanisole, 4-iodobenzonitrile or 2-bromopyridine.

108. The process according to claim 62, wherein the reaction is carried out in the presence of a base.

109. The process according to claim 108, wherein the base is: alkali metal carbonates, bicarbonates, hydroxides, alkali metal hydrides, alkali metal alcoholates, or tertiary amines.

110. The process according to claim 62, wherein the reaction is carried out in the presence of an organic solvent.

110. The process according to claim 110, wherein the organic solvent is: dimethylsulphoxide (DMSO); hexamethylphosphotriamide (HMPT); tetramethylurea; nitro compounds; aliphatic nitrites; aromatic nitrites, acetonitrile; tetramethylene sulphone; organic carbonates; alkyl esters; halogenated aromatic hydrocarbons, chlorobenzene, toluene, pyridine, picoline or quinolines.

111. The process according to claim 62, wherein the temperature of the arylation or vinylation or alkynation reaction is in the range 0° C. to 120° C.

112. The process according to claim 62, wherein the copper catalyst is: cuprous bromide, cupric bromide, cuprous iodide, cupric iodide, cupric chloride, basic copper (II) carbonate, cuprous nitrate, cupric nitrate, cuprous sulphate, cupric sulphate, cuprous sulphite, cuprous oxide, cuprous acetate, cupric acetate, cupric trifluoromethylsulphonate, cupric hydroxide, copper (1) methylate, copper (II) methyate or chlorocupric methylate with formula ClCuOCH₃.

113. The process according to claim 112, wherein the copper catalyst is cuprous chloride, cupric chloride, cuprous bromide, cupric bromide, cuprous oxide or cupric oxide.

114. The process according to claim 62, wherein the catalyst further comprises a metallic element M selected from groups (VIII), (IB) or (IIB) of the periodic table.

115. The process according to claim 114, wherein the metallic element M is silver, palladium, cobalt, nickel, iron or zinc.

116. The process according to claim 62, wherein the ligand is added concomitantly with at least one compound supplying the metallic copper.

117. The process according to claim 62, wherein the catalyst is a metallic complex prepared extemporaneously, by reacting at least one compound supplying the metallic elemental copper and a ligand.

118. The process according to claim 62, wherein the metallic complex is prepared at the start of the reaction from the ligand and the compound supplying the metallic elemental copper.

119. A metallic copper-based complex and its optically active forms having formula:
CuL₄X (C)
wherein:
X represents a halogen atom; and
L₄ represents a ligand having formula (Ib₁) or (Ib₂) in which ψ has the meaning given in said formulae, R_{b} represents a hydrogen atom or a methyl group and Rₐ represents a pyridyl group with formula: 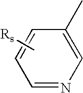
in which R_{S} has the meaning given above for formulae (F₀).

120. A metallic complex according to claim 119, wherein
L₄ represents a ligand having formula (Ib₁) in which ψ represents a urea group or one of groups (F₆) or (F₇) and Rₐ represents a pyridyl group as defined above, in which R_{S} has the meaning given for formulae (F₅); and
X a chlorine, bromine or iodine atom.

121. A metallic complex according to claim 119, having the formula:

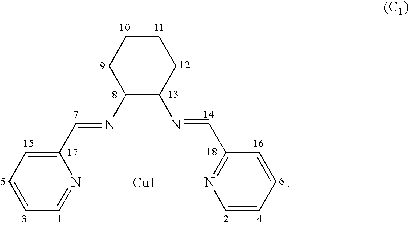

122. Novel compounds with formulae: 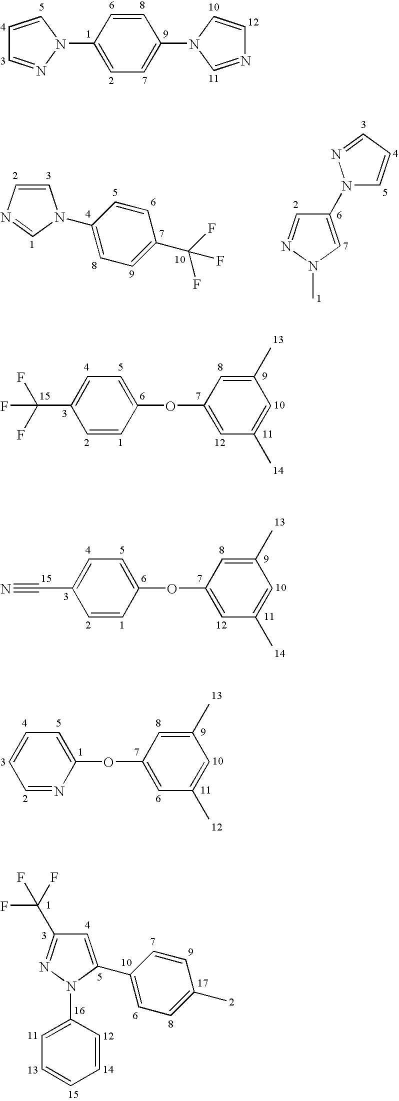 [chemical structure]
